(19) United States
(12) Patent Application Publication
(10) Pub. No.: US 2022/0290116 A1
(43) Pub. Date: Sep. 15, 2022

(54) CYSTATHIONINE BETA-SYNTHASE ENZYME THERAPY FOR TREATMENT OF ELEVATED HOMOCYSTEINE LEVELS

(71) Applicant: Travere Therapeutics Switzerland GmbH, Rapperswil-Jona (CH)

(72) Inventors: Marcia SELLOS-MOURA, West Newbury, MA (US); Erez Moshe BUBLIL, Ets Efraim (IL); Frank GLAVIN, Belmont, MA (US)

(21) Appl. No.: 17/639,509

(22) PCT Filed: Sep. 3, 2020
(86) PCT No.: PCT/US2020/049156

§ 371 Date: Mar. 1, 2022

Related U.S. Application Data
(60) Provisional application No. 62/983,862, filed on Mar. 2, 2020.
(60) Provisional application No. 62/895,230, filed on Sep. 3, 2019.

(51) Int. Cl. C12N 9/88; A61K 47/60; A61P 21/00; A61P 25/00; A61P 3/00; A61P 9/00

(52) U.S. Cl. CPC C12N 9/88; A61K 47/60; A61P 21/00; A61P 25/00; A61P 3/00; A61P 9/00; C12Y 402/01022; A61K 38/00

(57) ABSTRACT

The present disclosure provides methods for treating homocystinuria or elevated homocysteine levels in subjects, including methods of improving cognitive function and ameliorating skeletal fragility, and methods of stratifying patient populations to determine disease progression or severity and/or to determine treatment regimens. In some embodiments, the methods of improving cognitive function in a subject having elevated total plasma homocysteine (tHcy) levels further comprise providing a cognitive or behavioral intervention.

### CROSS REFERENCE TO RELATED APPLICATIONS

This application claims priority to U.S. provisional patent application No. 62/895,230 filed Sep. 3, 2019 and U.S. provisional patent application No. 62/983,862 filed Mar. 2, 2020, the contents of each of which are herein incorporated by reference in their entirety.

### REFERENCE TO THE SEQUENCE LISTING

The present application is being filed along with a Sequence Listing in electronic format. The Sequence Listing file, entitled 2089_1006PCT_SL.txt, was created on Sep. 3, 2020, and is 18,341 bytes in size. The information in electronic format of the Sequence Listing is incorporated herein by reference in its entirety.

### FIELD OF THE DISCLOSURE

The disclosure relates to compositions and methods for enzyme therapy for treatment of homocystinuria and for treatment of conditions associated with elevated homocysteine levels using the drug product described herein.

### BACKGROUND OF THE DISCLOSURE

Classical homocystinuria, referred to herein as HCU or HCU Type 1 and also known as cystathionine β-synthase deficient homocystinuria (CBSDH), is an orphan disease affecting both children and adults. HCU is a rare autosomal-recessive metabolic condition characterized by an excess of the compound homocysteine (Hcy) in the urine, tissues, and plasma, due to reduced or absence of activity of the cystathionine β-synthase (CBS) enzyme (see Kraus et al., In: Carmel R, Jacobsen D W, eds. Homocysteine in Health and Disease. Cambridge, United Kingdom: Cambridge University Press; 2001:223-243; Sacharow et al., Homocystinuria Caused by Cystathionine Beta-Synthase Deficiency. In: Adam M P, Ardinger H H, Pagon R A, Wallace S E, Bean L J H, Mefford H C, et al, eds. GeneReviews™ [Internet]. Seattle (WA): University of Washington, Seattle, 2017; each of which is incorporated by reference herein in its entirety).

The diagnosis of HCU may be confirmed by molecular genetic testing of the CBS gene as described in Sacharow et al. CBS is an enzyme in the metabolism of the sulfur amino acid methionine (Met), which is present in proteins in the diet (see Maclean et al. J Biol Chem. 2012; 287(38):31994-32005, which is hereby incorporated by reference in its entirety).

HCU may be suspected based on the following: 1) clinical findings including ectopia lentis (dislodgment of the lens in eye) and/or severe myopia, asthenic habitus (tall and slender), skeletal abnormalities, early onset osteoporosis, and/or thromboembolic events, unexplained developmental delay/intellectual disability; 2) newborn screening for hypermethioninaemia or specifically a positive family history for CBS deficiency may lead to pre-symptomatic patient identification; and 3) family history. There is considerable variability in all of these clinical signs and the age of symptom onset among patients. The current screening approaches usually fail to detect newborns with less severe CBS deficiency and only detect a minority of patients with more severe HCU (see, Huemer et al. J Inherit Metab Dis. 2015 November; 38(6):1007-19; Yap, Orphanet Encyclopedia [online serial]. 2005, pages 1-13; Schiff et al. Neuropediatrics. 2012 December; 43(6):295-304; each of which is hereby incorporated by reference in its entirety).

Outside the context of homocystinuria (that is, diagnosed or genetically defined HCU), elevated total plasma homocysteine levels have been associated with increased risk of osteoporosis and/or bone fracture (see van Meurs et al. N Engl J Med 2004; 350:2033-2041). Additionally, elevated tHcy is “a modifiable risk factor for development of cognitive decline, dementia, and Alzheimer's disease” (Smith, A. David, et al. “Homocysteine and dementia: an international consensus statement.” Journal of Alzheimer's Disease 62.2 (2018): 561-570).

Therefore, there is a need in the art for a methods of identifying, stratifying, and treating subjects having CBS deficiency and/or elevated tHcy levels who do not have diagnosed or genetically defined HCU.

### SUMMARY OF THE DISCLOSURE

Provided herein are methods of improving cognitive function in a subject having elevated total plasma homocysteine (tHcy) levels, the methods comprising administering to the subject a therapeutically effective amount of a pharmaceutical formulation comprising: a drug substance comprising an isolated cystathionine β-synthase (CBS) protein comprising SEQ ID NO: 1; a PEG molecule covalently bound to the CBS protein; and a pharmaceutically acceptable excipient, diluent, or adjuvant.

In some embodiments, the methods of improving cognitive function in a subject having elevated total plasma homocysteine (tHcy) levels further comprise providing a cognitive or behavioral intervention.

In some embodiments, the cognitive or behavioral intervention comprises behavioral parent training (BPT) or behavioral classroom management (BCM).

Also provided are methods of reducing skeletal fragility in a subject having elevated total plasma homocysteine (tHcy) levels, the method comprising administering to the subject a therapeutically effective amount of a pharmaceutical formulation comprising: a drug substance comprising an isolated cystathionine β-synthase (CBS) protein comprising SEQ ID NO: 1; a PEG molecule covalently bound to the CBS protein; and a pharmaceutically acceptable excipient, diluent, or adjuvant.

In some embodiments, skeletal fragility of the subject is assessed by bone mineral density determination.

In some embodiments, the PEG molecule is covalently bound to the CBS protein is ME-200GS.

In some embodiments, the therapeutically effective amount comprises a dosage of about 0.25 mg/kg to about 10 mg/kg of the drug substance. In some embodiments, the therapeutically effective amount comprises a dosage of about 0.25 mg/kg to about 10 mg/kg twice daily of the drug substance.

In some embodiments, the dosage is about 0.33 mg/kg of the drug substance. In some embodiments, the dosage is about 0.33 mg/kg twice daily of the drug substance.

In some embodiments, the dosage is about 0.66 mg/kg of the drug substance. In some embodiments, the dosage is about 0.66 mg/kg twice daily of the drug substance.

In some embodiments, the dosage is about 1.0 mg/kg of the drug substance. In some embodiments, the dosage is about 1.0 mg/kg twice daily of the drug substance.

In some embodiments, the dosage is about 1.5 mg/kg of the drug substance. In some embodiments, the dosage is about 1.5 mg/kg twice daily of the drug substance.

In some embodiments, the dosage is self-administered.

In some embodiments, the methods further comprise administering one or more of vitamin B6, and betaine, to the subject.

In some embodiments, the subject is on a methionine (Met)-restricted diet.

In some embodiments, the methods further comprise terminating or relaxing the methionine restricted diet. For example, upon administering the pharmaceutically effective amount of the drug product described herein, the subject may be removed from a methionine-restricted diet, or may have a relaxed methionine-restricted diet.

In some embodiments, the methods further comprise administering an anti-platelet agent.

In some embodiments, the anti-platelet agent is a warfarin blood thinner or an anti-coagulation agent.

In some embodiments, the administering the therapeutically effective amount of a pharmaceutical formulation occurs about once every 3 days.

In some embodiments, administering the therapeutically effective amount of a pharmaceutical formulation occurs about once every 2 days. In some embodiments, administering the therapeutically effective amount of a pharmaceutical formulation occurs about once per day.

In some embodiments, administering the therapeutically effective amount of a pharmaceutical formulation occurs about twice per day.

In some embodiments, administering the therapeutically effective amount of a pharmaceutical formulation occurs about once per week.

In some embodiments, administering the therapeutically effective amount of a pharmaceutical formulation occurs about twice per week.

In some embodiments, administering the therapeutically effective amount of a pharmaceutical formulation is repeated for about 6 weeks.

In some embodiments, administering the therapeutically effective amount of a pharmaceutical formulation is repeated for about 3 months.

In some embodiments, administering the therapeutically effective amount of a pharmaceutical formulation is repeated for about 6 months.

In some embodiments, administering the therapeutically effective amount of a pharmaceutical formulation is repeated for longer than 6 months.

In some embodiments, administering the therapeutically effective amount of a pharmaceutical formulation is repeated for the remaining life span of the subject.

In some embodiments, the elevated total plasma homocysteine (tHcy) levels in the subject comprise tHcy levels greater than about 5 μmol/L.

In some embodiments, the elevated total plasma homocysteine (tHcy) levels in the subject comprise tHcy levels greater than about 10 μmol/L.

In some embodiments, the elevated total plasma homocysteine (tHcy) levels in the subject comprise tHcy levels greater than about 15 μmol/L.

In some embodiments, the subject having elevated total plasma homocysteine (tHcy) levels is a genetically-defined HCU patient.

In some embodiments, the subject having elevated total plasma homocysteine (tHcy) levels is a non-genetically defined patient having elevated tHcy levels or having CBS deficiency.

Also provided are methods of treating CBS deficiency in a subject, the method comprising: determining a level of a metabolic indicator of disease severity or disease progression in the subject; and administering to the subject a therapeutically effective amount of a pharmaceutical formulation comprising: (i) a drug substance comprising an isolated cystathionine β-synthase (CBS) protein comprising SEQ ID NO: 1; (ii) a PEG molecule covalently bound to the CBS protein; and (iii) a pharmaceutically acceptable excipient, diluent, or adjuvant; wherein the subject is a genetically defined HCU patient or a non-genetically defined patient having elevated tHcy levels or having CBS deficiency, and wherein the therapeutically effective amount of the pharmaceutical formulation comprises a dosage of the drug substance adjusted according to the level of the metabolic indicator of disease severity or disease progression in the subject.

In some embodiments, determining a level of a metabolic indicator of disease severity or disease progression in the subject comprises obtaining a blood or a blood plasma sample from the subject, measuring a level of one or more metabolic indicator of disease severity or disease progression in the sample, and comparing the level of the one or more metabolic indicator of disease severity or disease progression to a level of the same metabolic indicator in a control sample from a healthy subject.

In some embodiments, the dosage of the drug substance adjusted according to the level of the metabolic indicator of disease severity or disease progression in the subject comprises a low dose, a medium dose, or a high dose of 20NHS PEG-CBS.

In some embodiments, a low dose of 20NHS PEG-CBS comprises about 0.25 mg/kg to about 1.0 mg/kg 20NHS PEG-CBS once or twice daily.

In some embodiments, a medium dose of 20NHS PEG-CBS comprises about 0.5 mg/kg to about 1.5 mg/kg 20NHS PEG-CBS once or twice daily.

In some embodiments, a high dose of 20NHS PEG-CBS comprises about 1 mg/kg to about 2 mg/kg 20NHS PEG-CBS once or twice daily.

In some embodiments, a high dose of 20NHS PEG-CBS comprises about 2 mg/kg to about 10 mg/kg 20NHS PEG-CBS once or twice daily.

In some embodiments, the metabolic indicator of disease severity or disease progression is total homocysteine (tHcy), methionine, creatinine, C-reactive protein, dimethylglycine, alanine aminotransferase, Protein C, aspartate aminotransferase (AST), anti-thrombin III, and/or apolipoprotein A.

In some embodiments, the metabolic indicator of disease severity or progression is tHcy and a dosage of 20NHS PEG-CBS is administered to the subject according to an elevated-low, elevated-medium, or elevated-high tHcy level.

Also described herein are methods of treating a subject having or suspected of having homocystinuria comprising: measuring a level of one or more of creatinine, high sensitivity C-reactive protein, fibrinogen, or Protein C activity in the subject; comparing the one or more levels against a known range of values for the one or more levels in a population of subjects known to not have homocystinuria or a population of subjects known to have homocystinuria, or both; evaluating disease progression or disease severity in the subject according to the one or more levels measured in the subject; adjusting a dosage of enzyme therapy for the subject according to the disease progression or disease severity; and administering the enzyme therapy to the subject. In these methods, administering the enzyme therapy comprises administering a drug substance comprising an isolated cystathionine β-synthase (CBS) protein comprising SEQ ID NO: 1 or consisting of SEQ ID NO: 1, wherein the CBS protein has a PEG molecule covalently bound to the CBS protein.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 is a chart showing number of patients with high, normal, or low laboratory values for certain biological markers tested in a natural history study of HCU patients. In FIG. 1, ALT-SGPT=alanine aminotransferase−serum glutamic-pyruvic transaminase; AST-SGOT=aspartate aminotransferase−serum glutamic-oxaloacetic transaminase; DMG=dimethylglycine; hsCRP=“high sensitivity” C-reactive protein; P1NP=procollagen type 1 N-terminal propeptide.

FIG. 2 shows correlations of plasma tHcy levels ≤100 μM and >100 μM on skeletal fragility.

FIG. 3 shows NIH Toolbox median and quartile scores for tested cognitive functions. Te box-plot boxes encompass the 25th percentile to the 75th percentile of the data, with the median value represented as the horizontal line in the box. Whiskers represent the data minimum and maximum. Crystalized Cognition Composite=area of cognition that reflects past learning and knowledge. Fluid Cognition Composite=area of cognition that reflects the ability to learn, solve novel problems, and use memory.

FIG. 4 shows the effect of tHcy levels on cognitive function.

### DETAILED DESCRIPTION

### I. Introduction

Cystathionine beta synthase deficient homocystinuria (HCU) is characterized by increased levels of plasma homocysteine (Hcy), together with high levels of Met and decreased concentrations of cysteine (Cys) (see Yap S. Homocystinuria due to cystathionine beta-synthase deficiency. Orphanet Encyclopaedia [serial online] 2005; Morris et al. J Inherit Metab Dis 2017; 40:49-74; NORD, Kraus J P. Homocystinuria due to cystathionine beta-synthase deficiency. NORD [serial online] 2017; each of which is hereby incorporated by reference in its entirety). To date, more than 180 different mutations of the CBS gene associated with HCU have been identified (see Human Genome Mutation Database. 2017. Ref Type: Online Source. Available at hgmd.cfac.uk/ac/index.php, which is hereby incorporated by reference in its entirety). Homocysteine (Hcy) is a naturally occurring amino acid which, together with serine, serves as a substrate for the CBS enzyme. CBS governs the unidirectional flow of sulphur from methionine (Met) to cysteine (Cys) by operating at the intersection of the transmethylation, transsulfuration, and remethylation pathways (see Maclean et al. J Biol Chem. 2012; 287(38):31994-32005, which is hereby incorporated by reference in its entirety). Native CBS is activated by the binding of the allosteric activator S-adenosylmethionine (SAM) and catalyzes a β-replacement reaction in which serine condenses with Hcy in a pyridoxal-5′-phosphate (pyridoxine, or vitamin B6)-dependent manner to form cystathionine (Cth). Cystathionine γ-lyase (CGL), operating downstream of CBS, uses Cth as a substrate to generate Cys. Thus, proper function of CBS is important for the regulation of the metabolism of Hcy, Met, and Cys.

Normal total homocysteine (tHcy) levels vary with age, sex, and nutritional status, but typically range between 4.5 and 11 μmol/L (see Quest Diagnostics Reference Range; questdiagnostics.com, which is hereby incorporated by reference in its entirety). Men tend to have slightly (by 1 to 2 μmol/L) higher tHcy levels than women, and an approximate doubling of mean values is observed as patients aged from childhood to 80 years (see Refsum et al. Clin Chem 2004; 50:3-32, which is hereby incorporated by reference in its entirety). In folate-supplemented populations, the upper limit (97.5%) of tHcy levels is approximately 12 μmol/L in adults <65 years old and 16 μmol/L in adults greater than 65 years. Many HCU patients present with severe hyperhomocysteinemia with total tHcy levels greater than 100 μmol/L, while others exhibit elevations ranging from mild to several times normal (see Morris et al. J Inherit Metab Dis 2017; 40:49-74, which is hereby incorporated by reference in its entirety). tHcy levels have been observed to be highly correlated with the severity of the disease (see Yap et al. J Inherit Metab Dis 1998; 21:738-747, which is hereby incorporated by reference in its entirety).

The severity and presentation of HCU signs and symptoms vary widely among patients (see Karaca et al. Gene 2014; 534:197-203; Trondle et al. Acta Med Austriaca 2001; 28:145-151; Kluijtmans et al. Am J Hum Genet 1999; 65:59-67; each of which is hereby incorporated by reference in its entirety). Many patients present with severe hyper homocysteinemia with total homocysteine (tHcy) levels greater than 100 μmol/L, while others exhibit tHcy elevations ranging from mild to several times normal (see Morris et al. J Inherit Metab Dis 2017; 40:49-74, which is hereby incorporated by reference in its entirety). Significantly elevated tHcy levels generally correlate with a more severe presentation, while lower levels typically correlate with a milder form of the disease.

HCU is commonly classified according to whether the affected individual responds to total homocysteine (tHcy)-lowering treatment with pyridoxine (vitamin B6), a CBS enzyme cofactor required for normal CBS function (see Mudd et al. Am J Hum Genet 1985; 37: 1-31; Abbott et al. Am J Med Genet 1987; 26:959-969; which are hereby incorporated by reference in their entireties). In general, patients who are responsive to pyridoxine have lower tHcy levels, resulting in a milder form of the disorder. These patients may present later in life with only one or a few HCU symptoms, and many remain undiagnosed. Consequently, patients who are highly responsive to pyridoxine are believed to be under-represented in most studies.

Retrospective studies show that patients with the highest tHcy levels (treated or untreated) present with more severe symptoms and earlier in life (see Yap et al. J Inherit Metab Dis 1998; 21:738-747; Mudd et al. Am J Hum Genet 1985; 37:1-31, both of which are hereby incorporated by reference in its entirety). Untreated individuals with elevated tHcy levels typically present with failure to thrive, thromboembolism, severe myopia with subsequent dislocation of the optic lens, osteoporosis-type fractures, a Marfanoid habitus (in particular elongation of the long bones) and psychiatric abnormalities, including learning difficulties (see Yap S. Homocystinuria due to cystathionine beta-synthase deficiency. Orphanet Encyclopaedia [serial online] 2005; Morris et al. J Inherit Metab Dis 2017; 40:49-74; NORD, Kraus J P. Homocystinuria due to cystathionine beta-synthase deficiency. NORD [serial online] 2017, each of which is hereby incorporated by reference in its entirety). Some patients with elevated tHcy levels have a severe childhood-onset multisystem disease. Without treatment, life expectancy is markedly reduced in the more severe patients (see Morris et al. J Inherit Metab Dis 2017; 40:49-74, which is hereby incorporated by reference in its entirety).

Hcy-derived compounds, measured as tHcy, consist of free-thiol homocysteine (Hcy-SH or fHcy), disulfides (such as homocysteine-cysteine and homocysteine) and protein bound homocysteine (see Ueland; Nord Med 1989; 104:293-298; Mudd et al. N Engl J Med 1995; 333:325; Mudd et al. Arterioscler Thromb Vasc Biol 2000; 20:1704-1706; each of which is hereby incorporated by reference in its entirety). The distinction between the sulfhydryl form (homocysteine; Hcy) and the disulfide form (homocysteine) (see Yap S. Homocystinuria due to cystathionine beta-synthase deficiency. Orphanet Encyclopaedia [serial online] 2005, which is hereby incorporated by reference in its entirety) is important because many of the pathophysiological effects depend on the presence of the sulfhydryl group in Hcy (see Yap S. Homocystinuria due to cystathionine beta-synthase deficiency. Orphanet Encyclopaedia [serial online] 2005; Ueland et al. Nord Med 1989; 104:293-298; Mudd et al. N Engl J Med 1995; 333:325; each of which is hereby incorporated by reference in its entirety).

CBS is mainly expressed in liver, pancreas, kidney and brain (see Morris et al. J Inherit Metab Dis 2017; 40:49-74, which is hereby incorporated by reference in its entirety). The catalytic domain binds pyridoxal 5′-phosphate (the cofactor also known as pyridoxine or vitamin B6) and the regulatory domain binds SAM (an allosteric activator).

Insufficient levels of CBS enzymatic activity block the transsulfuration pathway at the first step, resulting in Hcy accumulation, elevated SAH and Met levels and decreased Cth and Cys levels. As the clinical evidence of these dysregulated Met metabolites reviewed herein demonstrates elevated Hcy (most often measured clinically as plasma tHcy) is most strongly implicated in the pathophysiology of HCU.

Higher than normal Hcy levels modify sulfhydryl groups on proteins, preventing correct protein crosslinking and leading to structural abnormalities across multiple body systems. Elevated Hcy levels also impair intracellular signaling, resulting in endothelial dysfunction and, ultimately, thromboembolism and vascular disease. In HCU, accumulation of Hcy, leads to ocular, skeletal, vascular, and psychological manifestations.

The diagnosis of HCU is sometimes confirmed by molecular genetic testing of the CBS gene (see Sacharow et al. Homocystinuria Caused by Cystathionine Beta-Synthase Deficiency. In: Adam M P, Ardinger H H, Pagon R A, Wallace S E, Bean L J H, Mefford H C, et al, editors. GeneReviews™ [Internet]. Seattle (WA): University of Washington, Seattle; 1993-2017, which is hereby incorporated by reference in its entirety). The current screening approaches usually fail to detect newborns with less severe CBS deficiency and only detect a minority of patients with more severe HCU (see Huemer et al. J Inherit Metab Dis. 2015 November; 38(6):1007-19; Yap, Orphanet Encyclopedia [online serial]. 2005, pages 1-13; Schiff et al. Neuropediatrics. 2012 December; 43(6):295-304).

The measurement of choice to determine Hcy levels in clinical samples is tHcy which includes free Hcy as well as Hcy bound to protein or in the form of disulfides. Normal tHcy levels vary with age, sex, and nutritional status, but typically range between 4.5 and 11 μM (QUEST DIAGNOSTICS™ reference range). Many HCU patients present with severe hyper-homocystinuria with total tHcy levels greater than 100 μM, while others exhibit elevations ranging from mild to several times normal (see Morris et al. J Inherit Metab Dis. 2017 January; 40(1):49-74, which is hereby incorporated by reference in its entirety). tHcy levels are highly correlated with the severity of the disease (see Yap et al. J Inherit Metab Dis 1998; 21: 738-47).

Studies have shown that a reduction of Hcy levels in HCU patients is correlated with less severe manifestations of the clinical symptoms (see Yap et al. J Inherit Metab Dis 1998; 21: 738-47; Yap et al. Arterioscler Thromb Vasc Biol. 2001 December; 21(12):2080-5; which are both hereby incorporated by reference herein). The pathways through which homocysteine levels cause damage to these systems have been widely described (see Ajith et al. Clin Chim Acta 2015; 450:316-321; Behera et al. J Cell Physiol 99999:1-6, 2016; Saha et al. FASEB J 2016; 30:441-456; which are hereby incorporated by reference in their entireties) and have led to studies investigating the role of Hcy in the general population, which have exposed the significant pathogenic role of Hcy in disease.

One goal of treatment with the drug product described herein is to increase CBS enzyme activity in circulation, resulting in improved metabolic control, thereby ameliorating the clinical manifestations of the disease and slowing or preventing further deterioration. High molecular-weight compounds, such as enzymes, have limited tissue penetration capability and are thus mainly present in the plasma. These proteins are typically maintained in the circulation for a short period of time, as they are removed from the bloodstream by several mechanisms (see Vugmeyster et al. World J Biol Chem. 2012; 3(4):73-92, which is hereby incorporated by reference in its entirety). Ideally, administered CBS would maintain high activity in plasma for sufficient time to have a steady effect on sulfur amino acid metabolism. This goal may be achieved by PEGylation, the addition of PEG moieties onto the surface of the protein. PEGylation of proteins is a strategy that has become widely accepted and has been shown to minimize proteolysis, immune response, and antigenicity, while increasing protein stability and size and reducing renal excretion (see Kang et al. 2009; 14(2):363-380, which is hereby incorporated by reference in its entirety). The drug product described herein is a PEGylated htCBS C15S enzyme formulated for administration to a subject and designed for prolonged systemic exposure.

### A. Clinical Manifestation of Homocystinuria

There is significant evidence indicating the causal effect of elevated tHcy levels and negative clinical outcomes in the four systems commonly affected in HCU patients (ocular, skeletal, cardiovascular, and neurologic). These data are further supplemented by studies in the general population demonstrating a strong relationship between mildly elevated levels of tHcy and negative outcomes.

1. Eyes

Abnormalities affecting the eyes may be an early clinical sign of HCU. Many individuals develop displacement of the lenses of the eyes away from the center of the eyeball (ectopia lentis). Affected individuals also usually develop severe myopia (short or near sightedness) and iridodonesis (quivering of the colored portion of the eye). Ectopia lentis and myopia usually develop after the first year of life and, in untreated individuals, before ten years of age (see Mudd et al. Am J Hum Genet 1985; 37: 1-31, which is hereby incorporated by reference in its entirety). Other eye abnormalities that occur less frequently include cataracts, degeneration of the optic nerve and glaucoma. Some individuals may have retinal detachment, which can cause blurred vision or the appearance of “floaters” in the field of vision (see Burke et al. Br J Ophthalmol, 1989; 73(6): 427-31, which is hereby incorporated by reference in its entirety).

Elevated Hcy levels are a strong and independent risk factor for ocular complications, in particular lens dislocation, in patients with HCU and in the general population (see Mudd et al. Am J Hum Genet 1985; 37: 1-31; Ajith et al. Clin Chim Acta 2015; 450:316-321; Mulvihill et al. J AAPOS 2001; 5:311-315; which are hereby incorporated by reference in their entireties). Even with prescribed pharmacologic and dietary interventions, the majority of HCU patients eventually present with ocular complications. Lowering Hcy levels has been observed to delay and perhaps prevent lens dislocation in HCU patients (see Yap et al. J Inherit Metab Dis 1998; 21: 738-47, which is hereby incorporated by reference in its entirety).

2. Central Nervous System

Developmental delay and learning problems, such as cognitive symptoms, may also be early signs of HCU occurring at one to three years of age (see Screening, Technology and Research in Genetics (STAR-G) Project. 2016. Homocystinuria. Available at newbornscreening.info; National Institutes of Health (NIH), US National Library of Medicine, Genetics Home Reference. Homocystinuria. 2016. ghr.nlm.nih.gov; which are hereby incorporated by reference in their entireties). Intelligence quotient (IQ) in individuals with HCU has been reported to range from 10 to 138. Patients with the highest tHcy levels are more likely to have lower IQs (with a mean IQ of 57 if untreated) compared to less severely affected patients (with a mean IQ of 79) (see Sacharow et al., Homocystinuria Caused by Cystathionine Beta-Synthase Deficiency. In: Adam M P, Ardinger H H, Pagon R A, Wallace S E, Bean L J H, Mefford H C, et al, editors. GeneReviews™ [Internet]. Seattle (WA): University of Washington, Seattle; 1993-2017, which is hereby incorporated by reference in its entirety).

Seizures occur in approximately 20% of untreated individuals with HCU (see Mudd et al. Am J Hum Genet 1985; 37: 1-31, which is hereby incorporated by reference in its entirety). Many individuals have psychiatric problems including personality disorder, anxiety, depression, obsessive compulsive behavior, and psychotic episodes (see Sacharow et al. 2017). Extrapyramidal signs such as dystonia may occur as well (see Screening, Technology and Research in Genetics (STAR-G) Project. 2016. Homocystinuria. Available at newbornscreening.info, which is hereby incorporated by reference in its entirety).

Studies have shown that early decreases in Hcy levels, induced by a low Met diet, folic acid/B vitamin supplementation and/or pyridoxine and betaine therapy can delay and sometimes prevent or reverse progression of various neurological disorders and allow normal IQ development in patients with HCU (see El Bashir et al. JIMD Rep 2015; 21:89-95; Yap et al. J Inherit Metab Dis 2001; 24:437-447; which are hereby incorporated by reference in their entireties). Associations between elevated levels of Hcy and central nervous system (CNS) symptoms, including cognitive symptoms, neurodegenerative diseases, seizures, dystonia, psychosis, cognitive impairment, dementia and depression, are well documented in HCU patients and in the general population (see Abbott et al. Am J Med Genet 1987; 26:959-969; Schimke et al. JAMA 1965; 193:711-719; Herrmann et al. Clin Chem Lab Med 2011; 49:435-441; which are hereby incorporated by reference in their entireties).

3. Skeletal System

Individuals with HCU frequently develop a variety of skeletal abnormalities. Affected individuals are often tall and slender with “marfanoid” habitus, which includes thinning and lengthening of the long bones (dolichostenomelia), knees that are bent inward so that they touch when the legs are straight (“knock knees” or genu valgum), a highly arched foot (pes cavus), abnormal sideways curvature of the spine (scoliosis), an abnormally protruding chest (pectus carinatum) or an abnormally sunken chest (pectus excavatum). By the teenage years, 50% of individuals show signs of osteoporosis (see Screening, Technology and Research in Genetics (STAR-G) Project. 2016. Homocystinuria. Available at newbornscreening.info, which is hereby incorporated by reference in its entirety). HCU is associated with an increased risk of osteoporotic fractures that partly can be attributed to low bone mineral density (see Mudd et al. Am J Hum Genet 1985; 37: 1-31; Weber et al. Mol Genet Metab 2016; 117:351-354; which are hereby incorporated by reference in their entireties).

In a study of 25 Irish patients with HCU followed over 25 years found that the risk of skeletal abnormalities was considerably lower in patients with good compliance with Hcy-lowering treatment compared with non-compliant patients (see Yap et al. J Inherit Metab Dis 1998; 21: 738-47, which is hereby incorporated by reference in its entirety).

4. Cardiovascular System

The relationship between HCU and vascular disease was first demonstrated in 1985 in an epidemiological study in patients with moderate to severely elevated Hcy levels due to homozygous HCU (see Mudd et al. Am J Hum Genet 1985; 37: 1-31, which is hereby incorporated by reference in its entirety). Thromboembolism is the most serious, often life threatening, complication of HCU, and can affect any vessel. It is the major cause of morbidity and early death in patients with HCU (see Yap et al. Arterioscler Thromb Vasc Biol. 2001 December; 21(12):2080-5, which is hereby incorporated by reference in its entirety).

The risk of thromboembolic events was approximately 25% by age 16 years and 50% by age 29 years. Several reports described how treatments decreasing tHcy levels significantly reduced the incidence of vascular events, the main cause of morbidity, in HCU patients (see Wilcken et al. J Inherit Metab Dis 1997; 20:295-300; Yap et al. Arterioscler Thromb Vasc Biol. 2001 December; 21(12):2080-5, which are hereby incorporated by reference in their entireties). Since then, a number of other studies demonstrated an increased risk of vascular events, in particular venous thrombosis, in HCU patients (see Kelly et al. Neurology 2003; 60:275-279; Magner et al. J Inherit Metab Dis 2011; 34:33-37; which are hereby incorporated by reference in their entireties).

5. Additional Manifestations

Although less common, several additional findings have been reported in patients with HCU including extremely fine, fragile skin, brittle hair, discoloration of the skin (hypopigmentation) and rashes on the cheeks (malar flushing). Some individuals may develop fatty changes in the liver, protrusion of part of the intestines through a tear in the abdominal wall (inguinal hernia) or inflammation of the pancreas. Abnormal front-to-back curvature of the spine (kyphosis) and a collapsed lung (spontaneous pneumothorax) have also been reported in individuals with HCU (see Yap; Orphanet Encyclopedia [online serial]. 2005, pages 1-13, which is hereby incorporated by reference in its entirety).

B. Elevated Homocysteine in Non-Genetically-Defined Patient Populations

Normal tHcy levels vary with age, sex, and nutritional status, but typically range between 4.5 and 11 μmol/L (Quest Diagnostics Reference Range, questdiagnostics.com). Men tend to have slightly (by 1 to 2 μmol/L) higher tHcy levels than women, and an approximate doubling of mean values is observed as patients age from childhood to 80 years (Refsum et al. Clin Chem 2004; 50:3-32, which is hereby incorporated by reference in its entirety). In folate-supplemented populations, the upper limit (97.5%) of tHcy levels is approximately 12 μmol/L in adults <65 years old and 16 μmol/L in adults greater than 65 years.

Subjects having tHcy levels above normal are at higher risk for developing complications related to elevated tHcy. For example, elevated total plasma homocysteine levels have been associated with increased risk of osteoporosis and/or bone fracture (see van Meurs et al. N Engl J Med 2004; 350:2033-2041; incorporated by reference herein in its entirety). Additionally, elevated tHcy is a known risk factor for cognitive symptoms including dementia and Alzheimer's disease (Smith, A. David, et al. “Homocysteine and dementia: an international consensus statement.” Journal of Alzheimer's Disease 62.2 (2018): 561-570; incorporated by reference herein in its entirety).

Thus, patients having elevated tHcy levels, i.e., greater than the normal range of between 4.5 and 11 μmol/L, can be treated using the compounds and methods described herein to alleviate skeletal, cardiovascular, and/or cognitive symptoms of elevated tHcy levels, irrespective of a genetically defined deficiency in cystathionine β-synthase.

### II. Compositions

A. Native Human CBS Enzyme

The CBS full native enzyme is a tetramer with four identical monomers, in which each monomer (63 kDa is size) is organized into three functional domains. The first is a N-terminal region of about 70 amino acids that binds heme and is thought to function in redox sensing and/or enzyme folding. The second is a central domain that contains the catalytic core and shows the fold of the type II family PLP (pyridoxal-5′-phosphate)-dependent enzymes. The coenzyme PLP is deeply buried in a cleft between the N- and C-terminal domains. The third region is the C-terminal regulatory domain, that consists of a tandem pair of CBS motifs that upon binding to S-adenosylmethionine (SAM) activates the enzyme. Removal of the regulatory region generates an enzyme which is constitutively active (see Miles et al. J Biol Chem. 2004 Jul. 16; 279(29):29871-4, which is hereby incorporated by reference in its entirety).

The pyridoxal-5′-phospahte (PLP)-dependent enzyme fold contains a heme group. It catalyzes the PLP-dependent beta-replacement reaction in which it condenses L-homocysteine with L-serine to form L-cystathionine. It is allosterically regulated by binding of S-adenosyl-L-methionine (Ado-Met) to the C-terminal regulatory domains, resulting in a conformational rearrangement of these domains and a release of an autoinhibitory block. CBS activation can also be achieved by totally removing the C-terminal regulatory domains, generating a dimeric form of the enzyme which is constitutively active (see Miles et al. J Biol Chem. 2004 Jul. 16; 279(29):29871-4; Ereno-Orbea et al. Proc Natl Acad Sci USA 111(37), E3845-3852 (2014); each of which is hereby incorporated by reference in its entirety).

The active substance in the drug product described herein is a recombinant human truncated CBS protein with a cysteine to serine substitution at amino acid position 15 of the protein (htCBS C15S) compared to the amino acid sequence of SEQ ID NO: 2 in the present sequence listing, which represents a native CBS protein, that has been modified by the addition of polyethylene glycol (PEG). The enzyme is also known as htCBS C15S. In certain embodiments, the drug substance htCBS C15S has the amino acid sequence of SEQ ID NO: 1.

This form of the enzyme has a high tendency toward aggregation, which poses a major constraint on manufacturing and production of human CBS (hCBS). PEGylated htCBS C15S (including “20NHS PEG-CBS” as defined herein) has been engineered to form dimers rather than tetramers, which are less susceptible to aggregation. High molecular-weight compounds, such as enzymes, are removed from circulation by degradation by proteolysis and various clearance mechanisms (see Vugmeyster et al. World J Biol Chem. 2012; 3(4):73-92, which is hereby incorporated by reference in its entirety). PEGylation is known to minimize proteolysis and immunogenicity, while increasing protein stability and reducing renal excretion (see Kang et al. 2009; 14(2):363-380, which is hereby incorporated by reference in its entirety). These structural modifications make the drug product described herein comprising PEGylated htCBS C15S a more suitable candidate than native hCBS as an enzyme therapy (ET) for HCU.

Native CBS is an intracellular enzyme, and no mechanism is known to exist for the uptake of the enzyme from the extracellular environment to its primary intracellular site of action, while PEGylated htCBS C15S acts extracellularly. Unlike native endogenous CBS, PEGylated htCBS C15S corrects the metabolic abnormalities by operating directly in circulation and indirectly in tissues and does so without requiring SAM for activation. The native hCBS enzyme is activated in cells upon binding of S-adenosyl methionine (SAM) to its C-terminal regulatory domain. However, SAM levels in circulation in both patients and healthy individuals are far below the levels required for CBS activation (see Stabler et al. Metabolism, 2002.51(8): p. 981-8, which is hereby incorporated by reference in its entirety). Therefore, administration of native CBS into the circulation would be ineffective, as CBS would not become activated. PEGylated htCBS C15S, although it remains in circulation and does not enter cells, has been engineered to bypass the need for SAM activation by the removal of the CBS C-terminal regulatory domain rendering the enzyme constitutively active.

B. Enzyme Therapy (ET)

PEGylated htCBS C15S is a PEGylated, truncated hCBS with a cysteine to serine substitution at position 15 for ET for the treatment of HCU and/or the treatment of elevated total plasma homocysteine in non-genetically defined patients. This modification optimizes the enzyme to form dimers rather than tetramers and is constitutively active.

PEGylated htCBS C15S supplements deficient CBS activity, thereby reducing plasma levels of homocysteine (Hcy) and methionine (Met), increasing cystathionine (Cth) levels, and normalizing cysteine (Cys) levels in models of HCU. Reduction of total Hcy (tHcy) levels is the current treatment target (see, Morris et al. J Inherit Metab Dis. 2017 January; 40(1):49-74, which is hereby incorporated by reference in its entirety) and is strongly correlated with amelioration of clinical (ocular, skeletal, vascular, and neurological) outcomes (Yap; Orphanet Encyclopedia [online serial]. 2005, pages 1-13, which is hereby incorporated by reference in its entirety).

PEG htCBS C15S reduces plasma levels of homocysteine (Hcy) in subjects with elevated Hcy levels, including non-genetically-defined subjects having elevated tHcy levels.PEGylated htCBS C15S is a recombinant form of the native human CBS enzyme, which is produced in E. coli bacteria. The DNA sequence of native human CBS (SEQ ID NO: 3 in the present sequence listing was genetically modified to remove the C-terminal regulatory region (amino acids 414-551) (SEQ ID NO: 4), forming the human truncated CBS. The DNA sequence of the human truncated CBS was further modified to introduce a point mutation of T→A at position 43 of the DNA coding region (corresponding to SEQ ID NO: 3), resulting in a cysteine to serine substitution at position 15 of the translated protein, generating the human truncated CBS C15S (htCBS C15S) (SEQ ID NO: 5). This change reduces aggregation and allows for batch to batch consistency compared to the native hCBS.

The enzyme is further modified in the E. coli bacteria during expression, resulting in a removal of the first Met from the protein as shown in SEQ ID NO: 1. After its purification, the htCBS C15S enzyme is further modified by PEGylation with N-hydroxylsuccinimide ester functionalized 20 kDa PEG moieties, which react with primary amines on the surface of the protein. An approximate average of 5.1 PEG molecules are attached to each monomeric unit of the enzyme yielding a heterogeneous dimeric product of mean molecular weight of 290 kDa.

C. PEGylation of htCBS C15S to Produce 20NHS PEG-CBS

ME-200GS (also referred to as methoxy-PEG-CO(CH₂)₃COO—NHS) is used herein to PEGylate htCBS C15S:

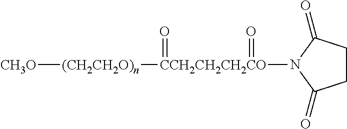

ME-200GS has a molecular weight of 20 kDa and a chemical name of α-Succinimidyloxyglutaryl-ω-methoxy, polyoxyethylene. ME-200GS targets free amines on the surface of htCBS C15S. An amide bond is formed between the PEG and the lysine residue on htCBS C15S. The resulting molecule is referred to throughout the disclosure as “20NHS PEG-CBS,” and is a PEGylated human cystathionine beta-synthase molecule that is truncated and that has a C15S mutation, as provided in SEQ ID NO: 1.

D. Post-Translational Modifications

Post-translational modifications can require additional bioprocess steps to separate modified and unmodified polypeptides, increasing costs and reducing efficiency of biologics production. Accordingly, in some embodiments, production of a polypeptide agent in a cell is enhanced by modulating the expression of a target gene encoding a protein that affects post-translational modification. In additional embodiments, biologics production is enhanced by modulating the expression of a first target gene encoding a protein that affects a first post-translational modification and modulating the expression of a second target gene encoding a protein that affects a second post-translational modification.

Additionally, proteins expressed in prokaryotic or eukaryotic cells can undergo several post-translational modifications that can impair production and/or the structure, biological activity, stability, homogeneity, and/or other properties of the biological product. Many of these modifications occur spontaneously during cell growth and polypeptide expression and can occur at several sites, including the peptide backbone, the amino acid side-chains, and the amino and/or carboxyl termini of a given polypeptide. In addition, a given polypeptide can comprise several different types of modifications. For example, proteins expressed in bacterial cells, such as E. coli, can be subject to acetylation, histone clipping, carboxylation, and/or deamidation (see Yang et al., PNAS 111 (52) E5633-E5642 (2014), which is hereby incorporated by reference in its entirety). For example, proteins expressed in avian and mammalian cells, such as Chinese hamster ovary (CHO) cells, can be subject to acetylation, carboxylation, gamma-carboxylation, histone clipping, deamidation, N-terminal glutamine cyclization and deamidation, and asparagine deamidation.

In some embodiments, protein production is enhanced by modulating expression of a target gene which encodes a protein involved in protein deamidation. Proteins can be deamidated via several pathways, including the cyclization and deamidation of N-terminal glutamine and deamidation of asparagine. Thus, in one embodiment, the protein involved in protein deamidation is N-terminal asparagine amidohydrolase. Protein deamidation can lead to altered structural properties, reduced potency, reduced biological activity, reduced efficacy, increased immunogenicity, and/or other undesirable properties and can be measured by several methods, including but not limited to, separations of proteins based on charge by, e.g., ion exchange chromatography, HPLC, isoelectric focusing, capillary electrophoresis, native gel electrophoresis, reversed-phase chromatography, hydrophobic interaction chromatography, affinity chromatography, mass spectrometry, or the use of L-isoaspartyl methyltransferase.

In some embodiments, the protein that affects protein secretion is a molecular chaperone selected from the group consisting of: Hsp40, HSP47 (also referred to as serpin peptidase inhibitor, clade H; heat shock protein 47), HSP60, Hsp70, HSP90, HSP100, protein disulfide isomerase, peptidyl prolyl isomerase, calnexin, Erp57 (protein disulfide isomerase family A, member 3), and BAG 1. In some embodiments, the protein that affects protein secretion is selected from the group consisting of γ-secretase, p115, a signal recognition particle (SRP) protein, secretin, and a kinase (e.g., MEK).

It is contemplated that further optimization could be achieved by systematically either adding or removing nucleotides to generate longer or shorter sequences and testing those sequences generated by walking a window of the longer or shorter size up or down the target RNA from that point. Coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

E. Stability

The drug substance or the drug product is stable at a variety of temperatures and storage conditions. In some embodiments, the drug substance or the drug product is stable when stored at −65° C. and −20° C. Alternatively, the drug substance or the drug product may be stable when stored at a temperature in a range of about 2° C. to about 8° C. Alternatively, the drug substance or the drug product may be stable when stored at a temperature in a range of 25° C.±2° C. For example, the drug substance or the drug product remains stable between 20° C. and 25° C. In certain embodiments, the drug substance or the drug product is stable under reducing conditions. In certain embodiments, the drug substance or the drug product is stable under non-reducing conditions. In some embodiments, the drug substance or the drug product remains stable for at least 2 days, at least 7 days, at least 1 month, at least 2 months, at least 3 months, at least 6 months, or at least 12 months. For example, the drug substance or the drug product remains stable during storage for about 2 days. For example, the drug substance or the drug product remains stable during storage for about 7 days. For example, the drug substance or the drug product remains stable during storage for about 1 month. For example, the drug substance or the drug product remains stable during storage for about 2 months. For example, the drug substance or the drug product remains stable during storage for about 3 months. For example, the drug substance or the drug product remains stable during storage for about 6 months. For example, the drug substance or the drug product remains stable during storage for about 12 months. For example, the drug substance or the drug product remains stable during storage for about 18 months.

In some embodiments, the drug substance or the drug product remains stable for storage at −65° C. for up to 18 months. In some embodiments, the drug substance or the drug product remains stable for storage between about 2° C. and about 8° C. for up to 3 months. In some embodiments, the drug substance or the drug product remains stable for storage at 25° C.±2° C. for up to 1 month.

In some embodiments, the drug substance or the drug product remains stable for at least 3 freeze and thaw cycles. In some embodiments, the drug substance or the drug product remains stable for up to 6 freeze and thaw cycles. For example, the drug substance or the drug product remains stable for 5 freeze and thaw cycles. In certain embodiments, the drug product is stable following ejection from a syringe.

### III. Pharmaceutical Compositions

The drug product described herein comprising PEGylated htCBS C15S is intended to restore metabolic control and ameliorate the clinical manifestations of the disease by reducing homocysteine levels, and normalizing cysteine levels in patients with HCU. htCBS C15S is manufactured by recombinant technology using E. coli BL21 (DE3) and is formulated as a sterile drug product in 15 mM Potassium phosphate, 8% (w/v) trehalose, pH 7.5. The drug product is intended for administration by subcutaneous (SC) injection.

PEGylated htCBS C15S activity in circulation improved or even entirely normalized the metabolite profiles in tissues as well (see WO 2017/083327, which is hereby incorporated by reference in its entirety). Therefore, the drug product does not necessarily need to be delivered into its native intracellular milieu.

The drug product reduces the accumulation of toxic Hcy in HCU patients; normalizes Cys levels in circulation; increases the levels of Cth in circulation; and/or prevents, delays, and/or reverses the onset of HCU manifestations. The drug product achieves at least one of these benefits while allowing the patients to enjoy normal diet. In fact, increased Cth activity even with a regular diet (e.g. 4.0 g/kg of MET) has been observed to be evidence of increased activity of the drug product and/or decreased renal elimination.

The molecular weight of the drug substance calculated from isotopically averaged molecular weight from SEC/UV/MS is 45.290 kDa for the monomer and 90.58 kDa for the dimer.

All batches were a clear liquid that was practically free from visible particles and dark red in color. Additionally, SDS-PAGE performed in both reducing and non-reducing conditions and a Western blot provided results were consistent with each other for each batch. A distinctive, uniform, and constant pattern of the PEGylation variant was demonstrated using each of these methods. Concomitant medications, including anticoagulants, vitamin and mineral supplementation, betaine, antidepressants, may also be combined with the drug product described herein to enhance the efficacy of the pharmaceutical composition.

### IV. Formulations

For the above-mentioned therapeutic uses, the dosage administered will vary with the compound employed, the mode of administration, the treatment desired and the disorder indicated. For example, the daily dosage of the compound of the disclosure, if inhaled, may be in the range from 0.05 micrograms per kilogram body weight (μg/kg) to 100 micrograms per kilogram body weight (μg/kg). Alternatively, if the compound is administered orally, then the daily dosage of the compound of the disclosure may be in the range from 0.01 micrograms per kilogram body weight (μg/kg) to 100 milligrams per kilogram body weight (mg/kg).

The protein having an amino acid sequence SEQ ID NO: 1, which is PEGylated to form the drug substance described herein, may be used on its own but will generally be administered in the form of a pharmaceutical composition in association with a pharmaceutically acceptable adjuvant, diluent, or carrier. Therefore, the present disclosure further provides a pharmaceutical composition comprising the drug substance described herein in association with a pharmaceutically acceptable adjuvant, diluent or carrier.

Pharmaceutically acceptable adjuvants, diluents or carriers that may be used in the pharmaceutical compositions of the disclosure are those conventionally employed in the field of pharmaceutical formulation, and include, but are not limited to, sugars, sugar alcohols, starches, ion exchangers, alumina, aluminum stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat (lanolin).

The pharmaceutical compositions of the present disclosure may be administered orally, parenterally, by inhalation spray, rectally, nasally, buccally, vaginally or via an implanted reservoir. In one embodiment, the pharmaceutical composition may be administered orally. In one embodiment, the pharmaceutical composition may be administered subcutaneously. The pharmaceutical compositions of the disclosure may contain any conventional non-toxic pharmaceutically acceptable adjuvants, diluents or carriers. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable adjuvant, diluent, or carrier, for example, as a solution in 1,3-butanediol. Among the acceptable adjuvants, diluents, and carriers that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this disclosure may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of the disclosure may also be formulated in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to, cocoa butter, beeswax and polyethylene glycols.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions herein may be in a form to be administered through the circulatory system as shown in WO 2015/153102, WO 2016/183482, and WO 2018/009838, which are each hereby incorporated by reference in its entirety. The CBS protein may be encoded by a recombinant nucleic acid expressed by enucleated hematopoietic cells (EHCs), including erythroid or thromboid cells. For example, the erythroid cells are red blood cells, erythrocytes, or reticulocytes. For example, the thromboid cells are platelets. In certain embodiments, the encoded CBS protein is fused to a translated membrane-anchored polypeptide. In certain embodiments, the CBS protein is localized on the surface of the EHC. The CBS protein may be cleaved for activation of the enzyme in the extracellular space. Alternatively, the internally localized CBS protein may be released into the extracellular space by lysis of the EHC. Alternatively, the enzymatic target of the CBS protein may enter the EHC and then exits through the membrane after alteration. In certain embodiments, the CBS protein has an amino acid sequence of SEQ ID NO: 1, SEQ ID NO: 2, SEQ ID NO: 4 or SEQ ID NO: 5 in the present sequence listing.

Depending on the mode of administration, the pharmaceutical composition will comprise from 0.05 to 99% w (percent by weight), more specifically from 0.05 to 80% w, still more specifically from 0.10 to 70% w, and even more specifically from 0.10 to 50% w, of active ingredient, all percentages by weight being based on total composition.

Conventional procedures for the selection and preparation of suitable pharmaceutical formulations are described in, for example, “Pharmaceutics—The Science of Dosage Form Design”, M. E. Aulton, Churchill Livingstone, 1988, which is herein incorporated by reference in its entirety.In certain embodiments, the drug product is formulated for exposure of about 50 mU/μL in a subject. A lyophilized formulation may be used for administration to humans upon reconstitution.

A. Lyophilization

The pharmaceutical compositions may be in a lyophilized formulation. In some embodiments, the lyophilized formulation comprises the drug substance, a buffer, and an excipient. In certain embodiments, upon reconstitution of the lyophilized formulation in a suitable reconstitution buffer, water or any other pharmaceutically acceptable adjuvant, diluent or carrier, the concentration of the drug substance is between about 20-30 mg/ml. In some embodiments, the concentration of the drug substance is about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, or about 30 mg/ml. In some embodiments, the concentration of the drug substance is about 25.4 mg/ml. In certain embodiments, upon reconstitution of the lyophilized formulation in a suitable reconstitution buffer, water or any other pharmaceutically acceptable adjuvant, diluent or carrier, the buffer is potassium phosphate at a concentration of 15 mM. In certain embodiments, the excipient is trehalose at a concentration of 8% (w/v). In some embodiments, the formulation comprises sucrose such that, upon reconstitution of the lyophilized formulation in a suitable reconstitution buffer, water, or pharmaceutically acceptable adjuvant, diluent or carrier, the concentration of sucrose is 5%. In some embodiments, the collapse onset temperature (Tc,on) determined by freeze drying microscopy is −21° C. In some embodiments, the formulation has a pH of 7.5.

In some embodiments, the lyophilization process may be performed in 48 hours or less without the melting of the crystalline cake structure. The lyophilization process may be optimized to tune one or more of the following parameters or properties such as, but not limited to, (i) reduced reconstitution time of the lyophilized formulation (e.g. less than 1 minute), (ii) reduced viscosity to allow a more concentrated drug product, (iii) incorporation of an isotonic buffer to minimize pain to patients, and/or (iv) reduced de-PEGylation.

The lyophilized formulation may be prepared using the following protocol. Three days prior to the formulation preparation, the drug substance (stored at −80 ° C.) at 20-30 mg/ml or about 25 mg/ml is thawed for 72 hours at 2-8° C. in a refrigerator. After thawing, the drug substance is homogenized by gentle swirling. Dialysis is performed under controlled conditions at 2-8° C. for 24 hours. Dialysis cassettes with a 20-kDa cut-off are used and buffer is exchanged three times at a volume ratio of greater than or equal to 1:50 each time. The buffer is exchanged after 3 and 6 hours of total dialysis time. The last dialysis step is performed overnight. After dialysis, the formulation is recovered from the dialysis cassettes and filtered by using a 0.22-μm polyvinylidene difluoride (PVDF) filter. After filtration, vials are filled with a filling volume of 1.0 ml under laminar air-flow conditions.

Lyophilization is performed in an Epsilon 2-12D pilot scale freeze dryer (Martin Christ, Osterode, Germany). The chamber pressure is controlled by a capacitance gauge and regulated by a vacuum pump and a controlled nitrogen dosage.

After equilibration of the vials to 5° C., the vials are frozen to −45° C. and equilibrated for further 5 hours at −45° C. Shelf temperature is set to −15° C. for 31 hours in primary drying. Secondary drying is performed at a shelf temperature of 40° C. for 2.5 hours. At the end of the lyophilization process, the chamber is aerated with nitrogen to 800 mbar and the vials are stoppered by lifting the shelves. After stoppering, the chamber is aerated to atmospheric pressure with nitrogen.

During the freeze-drying process, product temperature, shelf temperature, condenser temperature and chamber pressure (capacitance and Pirani gauge) are monitored. The product temperature is monitored by Pt100 sensors (OMEGA™).

### V. Treatments of Diseases, Disorders, or Conditions

Individuals with HCU are typically asymptomatic at birth and, unless they are treated, symptoms appear in these individuals over time, some as early as infancy, many in childhood, and, as this is a spectrum disease, in some patients symptoms appear only in adulthood (see Yap, 2005; Mudd et al. Am J Hum Genet 1985; 37:1-31; Morris et al. Guidelines for the diagnosis and management of cystathionine beta-synthase deficiency. J Inherit Metab Dis 2017; 40:49-74; Mudd et al. In: Scriver C L, Beaudet A L, Sly W S, Valle D, eds. The Metabolic and Molecular Basis of Inherited Diseases. 7 ed. New York: McGraw Hill; 2001; 1279-1327, each of which is hereby incorporated by reference in its entirety). Four main organ systems are typically involved, ocular, skeletal and vascular systems, as well as the CNS. Other organs, such as liver, pancreas, gastrointestinal tract and skin, including hair follicles, may also be involved (see Mudd et al. Am J Hum Genet 1985; 37:1-31; Morris et al. J Inherit Metab Dis 2017; 40:49-74; Muacevic-Katanec et al. Coll Antropol 2011; 35:181-185; Suri et al. J Neurol Sci 2014; 347:305-309, each of which is hereby incorporated by reference in its entirety).

The accumulated data show that the reduction of Hcy levels can serve as an indicator for the successful application of enzyme therapy (ET) in HCU. It is consistent with the definition by the NIH-FDA Biomarker Working Group (see FDA-NIH Biomarker Working Group. BEST (Biomarkers, EndpointS, and other Tools) Resource [Internet]. Silver Spring (MD): Food and Drug Administration (US); 2016-. Reasonably Likely Surrogate Endpoint. 2017 Sep. 25. Co-published by National Institutes of Health (US), Bethesda (MD). ncbi.nlm.nih.gov/books/NBK326791/, which is hereby incorporated by reference in its entirety) of a “pharmacodynamic/response biomarker, whose level changes in response to exposure to a medical product . . . ” and, even further, of a marker closely linked, in the case of an ET for HCU, to the drug mechanism of action. Therefore, blood or plasma Hcy is not only a useful marker for pharmacodynamic studies but has also been previously recognized as a “reasonably likely surrogate endpoint” for homocystinuria (HCU).

In infants and children with HCU, the priority is to prevent complications associated with HCU and to ensure proper growth and development of normal intelligence (see Morris et al. J Inherit Metab Dis 2017; 40:49-74, which is hereby incorporated by reference in its entirety). In patients diagnosed later in life, the aims of treatment should be to prevent life-threatening thromboembolism and to minimize progression of already established complications. To address these goals, the biochemical abnormalities associated with HCU must be improved and, if possible, normalized (see Morris et al. J Inherit Metab Dis 2017; 40:49-74, which is hereby incorporated by reference in its entirety).

According to the 2016 Guidelines for the Diagnosis and Management of HCU, Hcy levels should be maintained as close to normal as possible. This is not typically possible in patients with HCU given available treatments and so aspirational targets are suggested, below 50 μmol/L in patients with pyridoxine-responsive HCU and below 100 μmol/L in non-pyridoxine responsive patients (see Morris et al. J Inherit Metab Dis 2017; 40:49-74, which is hereby incorporated by reference in its entirety). As noted earlier, non-pyridoxine responsive patients tend to have higher Hcy levels than pyridoxine responsive ones. Although two goals are recommended for patients with the same disease, these goals were designed to be achievable, rather than optimized, in order to minimize complications.

Overall, the effectiveness of long-term treatments required for managing HCU, especially because they most frequently depend upon dietary restrictions and supplementation, are subject to poor or inconsistent lifelong compliance. An ET for HCU would avoid many of these pitfalls. By compensating for the metabolic defect in HCU through a mechanism that should not require severe Met restriction or Cys supplementation, an ET would be expected to achieve more consistent Hcy lowering, while not dangerously elevating Met levels, and also allowing for relaxation or normalization of the diet.

There is presently no cure for HCU that corrects the underlying genetic causes of the condition, but the generally accepted therapeutic goal is to reduce tHcy levels as much as possible (see Morris et al. J Inherit Metab Dis. 2017 January; 40(1):49-74, which is hereby incorporated by reference in its entirety). Consequently, current therapy is directed at correcting the biochemical abnormalities thereby reducing the risk of adverse clinical manifestations of the disease. Hcy levels are seldom fully normalized by treatments currently available for patients with HCU.

A combination of strategies is required to achieve treatment targets in most patients. These treatment strategies include: 1) increasing residual CBS activity by administering pharmacologic doses of pyridoxine (vitamin B6, a cofactor for CBS, along with folic acid) to pyridoxine sensitive patients (see Yap et al. Arterioscler Thromb Vasc Biol. 2001 December; 21(12):2080-5, which is hereby incorporated by reference in its entirety); 2) decreasing the methionine load through severe dietary/protein restriction, while supplementing the diet with products beyond the metabolic block, and 3) enhancing alternative metabolic pathways to counter the effects of the CBS deficiency, e.g., administer betaine (a methyl donor) to enhance remethylation of Hcy to Met. In certain embodiments, folate supplementation and (if needed) vitamin B12 supplements are provided (see Morris et al. J Inherit Metab Dis. 2017 January; 40(1):49-74, which is hereby incorporated by reference in its entirety).

A. Current Therapies for HCU

In some embodiments, patients with HCU should receive adequate folate supplementation and (if needed) vitamin B12 supplements (see Morris et al. J Inherit Metab Dis 2017; 40:49-74, which is hereby incorporated by reference in its entirety). In addition, patients should be treated with pyridoxine therapy (if responsive), a Met-restricted, Cys-supplemented diet and/or betaine therapy. A combination of strategies is required to achieve treatment targets in most patients.

The most common prescribed treatment was a combination of diet and betaine, followed by betaine alone and diet alone (see Adam et al. Mol Genet Metab 2013; 110:454-459, which is hereby incorporated by reference in its entirety). As patients become older than 16, they are most often prescribed betaine only without diet, in recognition of the poor compliance of adult patients with the Met-restricted diet. However, compliance with betaine in adults is also poor. The median protein intake varied widely among patients and increased dramatically with age.

Consistent with these findings, a recent report stated that only four adult patients among 24 patients prescribed a low protein diet with specific HCU-appropriate amino acid supplementation followed this treatment (see Lorenzini et al. J Inherit Metab Dis. 2017 Oct. 4, which is hereby incorporated by reference in its entirety). Multiple HCU experts have described similarly wide variability in the United States (Orphan Technology Scientific Advice Board comprised of physicians who are US HCU experts). A study comparing tHcy values from untreated versus treated patients (25 and 93 patients, respectively) concluded that there were no significant differences between these two groups (tHcy ranges 15.7 to 281.4 and 4.8 to 312 μmol/L, respectively; median values 125.0 and 119.0 μmol/L, respectively) although the study provided no details about the patients' treatment regimens (see Stabler et al. ENID Rep 2013; 11:149-163, which is hereby incorporated by reference in its entirety). These results suggest all or a combination of the following conclusions: that patients either had a heterogeneous presentation of the disease, that standard treatments were ineffective, and/or that compliance with treatment was poor.

1. Pyridoxine

Upon diagnosis, patients are tested for responsiveness to pyridoxine, a co-factor of CBS. Administration of pyridoxine (Vitamin B6) at pharmacologic doses increases the residual activity of CBS in individuals who have been shown to be pyridoxine responsive. The definition of pyridoxine responsiveness varies widely from site to site, though the 2016 guideline, written as part of the European network and registry for homocystinuria and methylation defects (EHOD), defined pyridoxine responsiveness as a 20% reduction in tHcy levels within 6 weeks of pyridoxine exposure. Patients with severely elevated tHcy levels and patients with mildly or moderately elevated tHcy levels can both be defined as responsive despite presenting with very different tHcy levels. Furthermore, different treatment centers define pyridoxine responsiveness differently, and therefore classification of patients by tHcy levels, rather than by their pyridoxine responsiveness, is more appropriate and rigorous. In general, patients who are responsive to pyridoxine have some residual CBS activity and therefore lower tHcy levels resulting in less severe presentation.

Pyridoxine is generally considered to be safe in patients with HCU (see Yap; Orphanet Encyclopedia [online serial]. 2005, pages 1-13, which is hereby incorporated by reference in its entirety). Its most commonly reported adverse effects include peripheral neuropathy in patients treated with high doses defined as greater than 900 mg/day (see Schaumburg et al. N Engl J Med 1983; 309:445-448; Ludolph et al. Eur J Pediatr 1993; 152:271; which are hereby incorporated by reference in their entireties), apnea and unresponsiveness in neonates receiving pyridoxine at 500 mg/day (Mudd et al. Am J Hum Genet 1985; 37: 1-31, which is hereby incorporated by reference in its entirety), and rhabdomyolysis (see Shoji et al. J Inherit Metab Dis 1998; 21:439-440, which is hereby incorporated by reference in its entirety).

While pyridoxine treatment is widely used, it provides a modest decrease in tHcy levels, and most patients who are defined as responsive are not able to significantly reduce, let alone normalize, tHcy levels on pyridoxine alone, as their starting levels are many fold above normal (see Morris et al. J Inherit Metab Dis. 2017 January; 40(1):49-74, which is hereby incorporated by reference in its entirety). Besides long-term pyridoxine treatment, it is recommended that pyridoxine responsive patients HCU patients also receive folate and as required, vitamin B12 supplementation.

2. Dietary Restriction

Lifelong dietary restrictions have been recommended previously for all patients with HCU (see Morris et al. J Inherit Metab Dis. 2017 January; 40(1):49-74; Walter et al. Eur J Pediatr. 1998 April; 157 Suppl 2:S71-6; which are hereby incorporated by reference in their entireties). The recommended diet is extremely limited and is aimed at reducing Met intake by restriction of protein content.

The mainstay of the present therapy for HCU patients is a lifelong low protein diet including as little as 5 g of natural protein per day (www.hcunetworkamerica.org) supplemented with Met-free-L-amino acids and, in many cases, additional Cys (Yap et al. J Inherit Metab Dis 1998; 21: 738-47; Morris et al. J Inherit Metab Dis. 2017 January; 40(1):49-74; which are hereby incorporated by reference in their entireties) is given to supplement the diet. The severely restricted diet consists of low-methionine cereal-based foods, low-methionine fruits and vegetables, low-methionine medical foods, oils, and sugar. Foods such as meat, chicken, fish, eggs, milk, yogurt, cheese, soy products, nuts, legumes and many fruits and vegetables that contain moderate amounts of Met should be avoided. Since native CBS is a key enzyme in Met metabolism, the ingestion of Met, an essential amino acid found in many foods, results in elevated plasma concentrations of tHcy and reduced concentrations of the downstream metabolites Cth and Cys.

Prepared foods, baked goods, and packaged foods must be highly restricted, as they often contain milk, eggs or flour. The amount of protein needed and tolerated by each HCU patient is different and is likely to vary with time. This amount is adjusted according to tHcy levels which are monitored in frequent blood tests (ASIEM Low Protein Handbook for Homocystinuria). The majority of patients on dietary treatment also requires a daily consumption of a poorly palatable, Met-free synthetic amino acid formula to prevent secondary malnutrition and for proper growth in children, and in adults for proper nutrition (see Morris et al. J Inherit Metab Dis. 2017 January; 40(1):49-74; which is hereby incorporated by reference in its entirety).

Though dietary modifications with combination of vitamin supplementation can decrease tHcy levels to some extent in individuals fully compliant with the highly restrictive diet, the tHcy levels of most HCU patients remain several-fold to orders of magnitude above normal. For most individuals, it is highly challenging to achieve full lifelong compliance with dietary modifications and the resulting periods of poor metabolic control have cumulative deleterious effects (see Morris et al. J Inherit Metab Dis. 2017 January; 40(1):49-74; which is hereby incorporated by reference in its entirety). Compliance with diet is often poor and generally deteriorates further during adolescence and adulthood (see Walter et al. Eur J Pediatr. 1998 April; 157 Suppl 2:S71-6; Schiff et al. Neuropediatrics. 2012 December; 43(6):295-304; Garland et al. Paediatr Child Health. 1999 November; 4(8):557-62; which are hereby incorporated by reference in their entireties). Moreover, eating high Met foods does not elicit an immediate negative physical reaction, further compounding the difficulty with diet compliance (www.hcunetworkamerica.org). Met restriction is even more challenging in children because of the need to ensure sufficient Met to facilitate growth and development. The majority of patients on dietary treatment also requires a Cys-enriched, Met-free L-amino acid supplement for proper growth in children, and in adults for proper nutrition (see Morris et al. J Inherit Metab Dis 2017; 40:49-74, which is hereby incorporated by reference in its entirety).

A recent report stated that only four adult patients among 24 patients prescribed a low protein diet with specific HCU-appropriate amino acid supplementation followed this treatment (see Lorenzini et al. J Inherit Metab Dis. (2018) 41:109-115, which is hereby incorporated by reference in its entirety). Multiple HCU experts have described similarly wide variability in the United States.

3. Betaine Supplementation

The problems associated with therapies based largely on diet have necessitated other approaches for Hcy lowering, most notably supplementation with betaine (N,N,N-trimethylglycine, marketed as CYSTADANE™) administered at least twice per day. Betaine is seldom effective as a monotherapy (Sakamoto et al. Pediatr Int 2003; 45:333-338, which is hereby incorporated by reference in its entirety), and is normally used as an adjunct to pyridoxine and/or a Met-restricted diet (see Morris et al. J Inherit Metab Dis. 2017 January; 40(1):49-74; which is hereby incorporated by reference in its entirety).

Betaine does not address the underlying CBS deficiency but rather induces an alternate pathway, resulting in remethylation of Hcy back to Met and by correcting the partial misfolding of CBS mutants (see Kopecka et al. J Inherit Metab Dis 2011; 34:39-48, which is hereby incorporated by reference in its entirety). In the presence of betaine, the enzyme betaine homocysteine methyltransferase (BHMT) remethylates Hcy to Met (Singh et al. Genet Med 2004; 6:90-95, which is hereby incorporated by reference in its entirety), thus partially reducing Hcy levels while increasing already highly elevated Met levels. Metabolites downstream of CBS are not ameliorated by betaine administration and Cys supplementation may be necessary. Moreover, betaine treatment has been associated with cerebral white matter abnormalities—a sign of vascular damage in the brain (Prins et al. Nat Rev Neurol 2015; 11:157-165, which is hereby incorporated by reference in its entirety)—in patients with (Devlin et al. J Pediatr 2004,144:545-548; Yaghmai et al. Am J Med Genet 2002; 108:57-63; which are hereby incorporated by reference in their entireties) and without (Vatanavicharn et al. J Inherit Metab Dis 2008; 31 Suppl 3:477-481; Brenton et al. J Child Neurol 2014; 29:88-92; Sasai et al. Tohoku J Exp Med 2015; 237:323-327; which are hereby incorporated by reference in their entireties) acute cerebral edema. Although betaine treatment is known to exacerbate methionine accumulation, Schwann et al. have shown that methionine is the primary agent causing brain edema (see Schwahn, Bernd C., et al. JIMD reports 52.1 (2020): 3-10, incorporated herein by reference in its entirety).

Betaine may be unpalatable (Walter et al. Eur J Pediatr. 1998 April; 157 Suppl 2:S71-6, which is hereby incorporated by reference in its entirety) and result in unpleasant fishy body odor and/or breath (see Manning et al. JIMD Rep 2012; 5:71-75, which is hereby incorporated by reference in its entirety). Both effects potentially exacerbated by the requirement for high doses (greater than 6 g/day in adult and pediatric patients). Consequently, compliance is generally poor (see Adam et al. Mol Genet Metab. 2013 December; 110(4):454-9; Walter et al. Eur J Pediatr. 1998 April; 157 Suppl 2:S71-6; Sakamoto et al. Pediatr Int 2003; 45:333-338; which are hereby incorporated by reference in their entireties).

The pharmaceutical formulation of betaine, CYSTADANE™, was approved by the FDA in 2006 and is indicated to decrease elevated blood Hcy in homocystinuria disorders including CBS deficiency, 5,10-methylenetetrahydrofolate reductase (MTHFR) deficiency, and cobalamin cofactor metabolism (61) defects (see Recordati. CYSTADANE™ Product Information (PI). 2017. Ref Type: Online Source, which is hereby incorporated by reference in its entirety).

The largest survey to date on dietary practices among HCU pyridoxine non-responsive patients indicated that betaine was a common treatment choice, particularly in late diagnosed patients, adolescents and adults. The use of betaine as the primary therapy in 34% of patients without diet is believed to be due to lack of compliance with the diet, since there are no controlled studies examining betaine's long-term effectiveness when given without diet (see Adam et al. Mol Genet Metab 2013; 110:454-459, which is hereby incorporated by reference in its entirety). Indeed, a study in an HCU mouse model found that the ability of betaine treatment to significantly lower tHcy was decreased over time (see Maclean KN. Betaine treatment of cystathionine b-synthase-deficient homocystinuria; does it work and can it be improved? Dove press 2012; 2:23-33, which is hereby incorporated by reference in its entirety).

4. Anti-Platelet Therapies

In addition to Hcy-lowering therapies, patients with poorly controlled Hcy levels and/or those who have additional risk factors for thrombosis (e.g. Factor V Leiden, previous thrombosis and pregnancy), may benefit from treatment with anti-platelet agents (e.g. aspirin, dipyridamole or clopidogrel) (see Morris et al. J Inherit Metab Dis. 2017 January; 40(1):49-74; which is hereby incorporated by reference in its entirety). COUMADIN™ blood thinners may also be used in patients with previous venous thrombosis. However, anticoagulation agents are associated with an increased risk of cerebral hemorrhage and their use should be determined on an individual patient basis (see Morris et al. J Inherit Metab Dis. 2017 January; 40(1):49-74; which is hereby incorporated by reference in its entirety).

5. Clinical Outcomes with Current Therapies

The manifestations of HCU continue to progress with the classic clinical symptoms reaching varying degrees of disability and impact on the quality of life of affected individuals. Regardless of the individual's age of onset, the loss of biochemical control at any age is associated with the development of serious complications that can be life-threatening (see Walter et al. Eur J Pediatr. 1998 April; 157 Suppl 2:S71-6, which is hereby incorporated by reference in its entirety).

Treatment must be continued throughout life, as periods of poor metabolic control have cumulative deleterious effects that can lead to severe complications and premature death (see Morris et al. J Inherit Metab Dis. 2017 January; 40(1):49-74, which is hereby incorporated by reference in its entirety).

Without treatment, the prognosis of pyridoxine-unresponsive HCU is poor and patients' life expectancy is markedly reduced. No randomized controlled trials of dietary or other therapy of HCU have been conducted since the disease was first described in 1962 (see Carson et al. Arch Dis Child. 1969 June; 44(235):387-92; Gerritsen et al. Biochem Biophys Res Commun. 1962 Dec. 19; 9:493-6; which are hereby incorporated by reference in their entireties). However, several observational studies have been published.

An international study that documented the natural history of 629 untreated CBS patients showed that the risk of complications increases with age (see Mudd et al. Am J Hum Genet 1985; 37: 1-31, which is hereby incorporated by reference in its entirety). Treatment (pyridoxine, Met restricted diet) was observed to lower plasma tHcy levels and markedly reduced the risk of thromboembolic events and lens dislocation, although compliance with the restricted diet was observed to be poor.

Yap et al. (Arterioscler Thromb Vasc Biol. 2001 December; 21(12):2080-5) conducted an international multicenter study in 158 treated patients. The incidence of cardiovascular events was reduced markedly in this treated group when compared to the historical control data from Mudd et al. (Am J Hum Genet 1985; 37: 1-31, which is hereby incorporated by reference in its entirety). This apparent benefit was correlated with lower (but not normalized) tHcy plasma levels in the treated patients. It was noted that consistent compliance with the required regimen was very difficult.

Overall, while several HCU treatment strategies are available, they are unable to restore most patients to near normal tHcy levels. Moreover, their long-term effectiveness, are subject to poor or inconsistent lifelong compliance. Thus, consistent Hcy lowering is difficult to maintain in HCU patients. Treatment with the drug product described herein aims to avoid many of these pitfalls. By compensating for the metabolic defect in HCU through a mechanism that should not require severe Met restriction or Cys supplementation, enzyme therapy (ET) therapy is expected to achieve more consistent Hcy lowering, while not dangerously elevating Met levels.

B. Methods of Improving Cognitive Function

The present patent application is directed to methods of improving cognitive function in a subject having or suspected of having homocystinuria. The methods can comprise administering to the subject a therapeutically effective amount of a pharmaceutical formulation comprising: a drug substance comprising an isolated cystathionine β-synthase (CBS) protein (SEQ ID NO: 1); a PEG molecule covalently bound to the CBS protein; and a pharmaceutically acceptable excipient, diluent, or adjuvant. In some aspects, combining enzyme therapy comprising administration of a pharmaceutical formulation comprising htCBS C15S protein as described herein with one or more cognitive or behavioral intervention can provide a further improvement in cognitive function or behavior in a subject having or suspected of having homocystinuria. In some embodiments, neurobehavioral or cognitive treatment or intervention includes supports similar to those for other executive functioning problems, such as ADHD, including, for example, behavioral therapies such as behavioral parent training (BPT) and behavioral classroom management (BCM) (see, e.g., Pelham Jr, William E., and Gregory A. Fabiano. “Evidence-based psychosocial treatments for attention-deficit/hyperactivity disorder.” Journal of Clinical Child & Adolescent Psychology 37.1 (2008): 184-214; and Pfiffner, Linda J., and Lauren M. Haack. “Behavior management for school-aged children with ADHD.” Child and Adolescent Psychiatric Clinics 23.4 (2014): 731-746, the contents of each which are incorporated by reference herein in their entirety). Thus, the present disclosure provides combination therapies involving enzyme therapy as described herein combined with neurobehavioral or cognitive therapies to further improve cognitive function in subjects having or suspected of having homocystinuria. It is expected that such combination therapies will effectively improve patient cognitive function, including cognitive development in pediatric subjects, to a greater degree than either enzyme therapy or neurobehavioral/cognitive therapy alone.

The pharmaceutical formulations for use in the methods of improving cognitive function can comprise administering ME-200GS as the PEG molecule. The pharmaceutical formulations can comprise the drug substance in a concentration of about 25.4 mg/ml, optionally in 5% sucrose, about 15 mM potassium phosphate; and about 8% (w/v) trehalose. The pharmaceutical formulations can be lyophilized.

C. Treatment Methods Based on Metabolic Indicators

Also described herein are methods of treating a subject having or suspected of having homocystinuria comprising: measuring a level of one or more of creatinine, high sensitivity C-reactive protein, fibrinogen, or Protein C activity in the subject; comparing the one or more levels against a known range of values for the one or more levels in a population of subjects known to not have homocystinuria or a population of subjects known to have homocystinuria, or both; evaluating disease progression or disease severity in the subject according to the one or more levels measured in the subject; adjusting a dosage of enzyme therapy for the subject according to the disease progression or disease severity; and administering the enzyme therapy to the subject. In these methods, administering the enzyme therapy comprises administering a drug substance comprising an isolated cystathionine β-synthase (CBS) protein comprising SEQ ID NO: 1 or consisting of SEQ ID NO: 1, wherein the CBS protein has a PEG molecule covalently bound to the CBS protein.

In the methods described herein, methionine levels ≥600 μM can be used to identify and stratify subjects based on response to methionine restricted diet and/or betaine and/or vitamin supplementation in pediatric and adult HCU subjects. In some embodiments, methionine levels >1000 μM can be used to identify and stratify subjects based on response to methionine restricted diet and/or betaine and/or vitamin supplementation in pediatric and adult HCU subjects.

Dimethylglycine (DMG) levels above the upper limit of normal (ULN) can be used to identify and stratify subjects based on response to betaine in pediatric and adult HCU patients. Creatinine levels below the lower limit of normal (LLN) can be used to identify and stratify subjects that may benefit from enzyme therapy according to the methods described herein. In some embodiments, high sensitivity C-reactive protein (hsCRP) levels above the ULN can be used to identify and stratify subjects that may benefit from enzyme therapy according to the methods described herein. In some embodiments, low Protein C activity levels and/or low fibrinogen levels can be used to identify and stratify subjects that may benefit from enzyme therapy according to the methods described herein.

The methods described herein can further comprise molecular genetic testing of a CBS gene in the subject, wherein identification of a mutation in the CBS gene indicates a level of disease severity or confirms a diagnosis of homocystinuria. Non-limiting examples of CBS gene mutations are provided in Table 3.

D. Methods of Improving Skeletal Fragility

In some embodiments, the present application is directed to methods of reducing skeletal fragility in a subject having or suspected of having homocystinuria. The methods can comprise administering to the subject a therapeutically effective amount of a pharmaceutical formulation comprising: a drug substance comprising an isolated cystathionine β-synthase (CBS) protein (SEQ ID NO: 1); a PEG molecule covalently bound to the CBS protein; and a pharmaceutically acceptable excipient, diluent, or adjuvant.

The pharmaceutical formulations for use in the methods of improving skeletal fragility can comprise ME-200GS as the PEG molecule. The pharmaceutical formulations can comprise administering the drug substance in a concentration of about 25.4 mg/ml, optionally in 5% sucrose, about 15 mM potassium phosphate; and about 8% (w/v) trehalose. The pharmaceutical formulations can be lyophilized.

E. Methods of Improving Cardiovascular Symptoms

In some embodiments, the present application is directed to methods of improving cardiovascular symptoms and vascular complications as described herein in a subject having or suspected of having homocystinuria (see van Meurs et al. N Engl J Med 2004; 350:2033-2041; Hankey et al. Lancet 1999; 354:407-413; both of which are hereby incorporated by reference in their entirety). The methods can comprise administering to the subject a therapeutically effective amount of a pharmaceutical formulation comprising: a drug substance comprising an isolated cystathionine β-synthase (CBS) protein (SEQ ID NO: 1); a PEG molecule covalently bound to the CBS protein; and a pharmaceutically acceptable excipient, diluent, or adjuvant.

The pharmaceutical formulations for use in the methods of improving cardiovascular symptoms and vascular complications can comprise ME-200GS as the PEG molecule. The pharmaceutical formulations can comprise administering the drug substance in a concentration of about 25.4 mg/ml, optionally in 5% sucrose, about 15 mM potassium phosphate; and about 8% (w/v) trehalose. The pharmaceutical formulations can be lyophilized.

F. Methods of Treating Elevated tHcy Levels in Non-Genetically-Defined Patient Populations

The methods described herein comprise treating non-genetically defined patient populations having elevated tHcy levels. The terms “non-genetically-defined patient” or “non-genetically-defined subject” refer to one or more individuals not having or not diagnosed as having a genetically-defined deficiency in cystathionine β-synthase (e.g., not having a missense or loss-of-function mutation in one or more CBS gene allele).

Non-genetically defined subjects having elevated tHcy levels may present with or have increased risk for developing skeletal abnormalities, such as increased prevalence of bone fractures, reduced bone mineral density, and/or osteoporosis (see, for example, Filip, Alexandru, et al. “The Relationship between Homocysteine and Fragility Fractures-A Systematic Review.” Annual Research & Review in Biology (2017): 1-8; van Meurs et al. N Engl J Med 2004; 350:2033-2041; each of which is incorporated by reference herein in its entirety). Thus, some embodiments of the methods described herein provide for treatment or alleviation of skeletal symptoms associated with elevated tHcy levels by administering to a subject the pharmaceutical compositions or pharmaceutical formulations described herein. In some embodiments, administering the described pharmaceutical compositions or pharmaceutical formulations to non-genetically-defined subjects having elevated tHcy levels reduces tHcy levels in the subjects. In some embodiments, administering the described pharmaceutical compositions or pharmaceutical formulations to non-genetically-defined subjects having elevated tHcy levels alleviates skeletal symptoms associated with elevated tHcy levels in the subject, including risk for bone fracture, reduced bone mineral density, and/or osteoporosis.

Non-genetically defined subjects having elevated tHcy levels may present with or have increased risk for developing cognitive symptoms or cognitive abnormalities, such as increased prevalence cognitive decline, dementia, and/or Alzheimer's disease (see, for example, Smith, A. David, et al. “Homocysteine and dementia: an international consensus statement.” Journal of Alzheimer's Disease 62.2 (2018): 561-570; Smith, A. David, and Helga Refsum. “Homocysteine, B vitamins, and cognitive impairment.” Annual review of nutrition 36 (2016): 211-239; Setién-Suero, Esther, et al. “Homocysteine and cognition: a systematic review of 111 studies.” Neuroscience & Biobehavioral Reviews 69 (2016): 280-298; each of which is incorporated by reference herein in its entirety). Thus, some embodiments of the methods described herein provide for treatment or alleviation of cognitive symptoms associated with elevated tHcy levels by administering to a subject the pharmaceutical compositions or pharmaceutical formulations described herein. In some embodiments, the methods described herein provide for treatment or avoidance or slowing the progression of neurodegenerative disorders associated with elevated tHcy levels, including Alzheimer's disease (see Farina, Nicolas, et al. “Homocysteine concentrations in the cognitive progression of Alzheimer's disease.” Experimental Gerontology 99 (2017): 146-150, the disclosure of which is incorporated herein in its entirety). In some embodiments, administering the described pharmaceutical compositions or pharmaceutical formulations to non-genetically-defined subjects having elevated tHcy levels reduces tHcy levels in the subjects. In some embodiments, administering the described pharmaceutical compositions or pharmaceutical formulations to non-genetically-defined subjects having elevated tHcy levels alleviates cognitive symptoms associated with elevated tHcy levels in the subject, including cognitive decline, dementia, or Alzheimer's disease.

Non-genetically defined subjects having elevated tHcy levels may present with or have increased risk for developing vascular disease (see, for example, Kang, Soo-Sang, and Robert S. Rosenson. “Analytic approaches for the treatment of hyperhomocysteinemia and its impact on vascular disease.” Cardiovascular drugs and therapy 32.2 (2018): 233-240, the contents of which are incorporated by reference herein in their entirety). Thus, some embodiments of the methods described herein provide for treatment, alleviation, or avoidance of vascular disease.

Non-genetically defined subjects having elevated tHcy levels may present with or have increased risk for developing stroke (see, for example, Spence, J. David. “Homocysteine lowering for stroke prevention: unravelling the complexity of the evidence.” International Journal of Stroke 11.7 (2016): 744-747, the contents of which are incorporated by reference herein in their entirety). Thus, some embodiments of the methods described herein provide for treatment, alleviation, or avoidance of stroke.

Non-genetically defined subjects having elevated tHcy levels may present with or have increased risk for developing ocular disease (see, for example, Ajith, Thekkuttuparambil Ananthanarayanan. “Homocysteine in ocular diseases.” Clinica Chimica Acta 450 (2015): 316-321, the contents of which are incorporated by reference herein in their entirety). Thus, some embodiments of the methods described herein provide for treatment, alleviation, or avoidance of ocular disease.

Elevated tHcy levels may affect a wide range of metabolic and physiological systems. Accordingly, non-genetically defined subjects may present with or have increased risk for developing various phenotypic outcomes, including skeletal symptoms, cognitive symptoms, ocular symptoms, cardiovascular symptoms, and reproductive symptoms, among others.

For example, non-genetically defined subjects having elevated tHcy levels may present with or have increased risk for miscarriage (see Cavallé-Busquets, Pere, et al. “Moderately elevated first trimester fasting plasma total homocysteine is associated with increased probability of miscarriage. The Reus-Tarragona Birth Cohort Study.” Biochimie (2020), the disclosure of which is incorporated herein in its entirety). Accordingly, the methods described herein provide for avoidance of miscarriage in women with moderately elevated first trimester tHcy levels.

Non-genetically defined subjects, including children and adolescents, having elevated tHcy levels may present with or have increased risk for anxiety and depression (see Folstein, Marshal, et al. “The homocysteine hypothesis of depression.” American Journal of Psychiatry 164.6 (2007): 861-867; Chung, Kuo-Hsuan, Hung-Yi Chiou, and Yi-Hua Chen. “Associations between serum homocysteine levels and anxiety and depression among children and adolescents in Taiwan.” Scientific reports 7.1 (2017): 1-7; see also Mech, Arnold W., and Andrew Farah. “Correlation of clinical response with homocysteine reduction during therapy with reduced B vitamins in patients with MDD who are positive for MTHFR C677T or A1298C polymorphism: a randomized, double-blind, placebo-controlled study.” The Journal of clinical psychiatry 77.5 (2016): 668-671; the disclosures of which are incorporated herein in their entirety). Accordingly, the methods described herein provide for avoidance of anxiety or depression in children and adolescents with elevated first trimester tHcy levels.

Non-genetically defined subjects having elevated tHcy levels may present with or have increased risk for renal complications and/or cardiovascular disease and related conditions including ischemic heart disease and stroke (see van Guldener, Coen. “Why is homocysteine elevated in renal failure and what can be expected from homocysteine-lowering?.” Nephrology Dialysis Transplantation 21.5 (2006): 1161-1166; Clarke, Robert, Sarah Lewington, and Martin Landray. “Homocysteine, renal function, and risk of cardiovascular disease.” Kidney international 63 (2003): S131-S133; Yang, Hui-Fang, et al. “Does serum homocysteine explain the connection between sexual frequency and cardiovascular risk?.” The Journal of Sexual Medicine 14.7 (2017): 910-917; the disclosures of which are incorporated herein in their entirety). Accordingly, the methods described herein provide for treatment or avoidance of renal complications, including for example renal failure and chronic kidney disease, and cardiovascular disease in subjects having elevated tHcy levels.

### VI. Dosing and Administration

In certain embodiments, the drug product may be administered to a subject by subcutaneous (SC), intravenous (IV) or intraperitoneal (IP) injection. In one embodiment, the drug product may be administered to a subject 1, 2, 3, 4, 5, 6, 7, 8, 9, 10, 11, 12, 13, 14, 15, 16, 17, 18, 19, or 20 time(s). In another embodiment, the drug product is administered more than 20 times. In another embodiment, the drug product is administered more than 100 times. Alternatively, the drug product may be administered for the remaining life span of the subject.

In certain embodiments, the administration of the drug product may be repeated every 12 hours, 13 hours, 14 hours, 15 hours, 16 hours, 17 hours, 18 hours, 19 hours, 20 hours, 21 hours, 22 hours, 23 hours, daily, 2 days, 3 days, 4 days, 5 days, 6 days, week, 2 weeks, 3 weeks, and month. In certain embodiments, administration of the drug product is performed once every 3 days, once every 2 days, or once per day.

In certain embodiments, the administration of the drug product may be a series of doses which are minutes, hours, days or weeks apart. For example, the number of doses in a series may be 1, 2, 3, 4, 5, or 6. As a non-limiting example, a subject is administered 3 doses 24 hours apart. As another non-limiting example, a subject is administered 5 doses 12 hours apart. The subject may be a human.

In certain embodiments, the administration of the drug product may follow a dosing schedule of a series of doses that has a gap between the first series and the second series of doses. The gap between the doses may be 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, day, 2 days, 3 days, 4 days, 5 days, 6 days, a week, 2 weeks, 3 weeks, monthly, 2 months, 3 months, 4 months, 5 months, 6 months, 7 months, 8 months, 9 months, 10 months, 11 months, 12 months, 13 months, 14 months, 15 months, 16 months, 17 months, or 18 months. The number of doses in a series may be 2, 3, 4, 5 or 6. As a non-limiting example, a subject may be administered a first series of 5 doses 12 hours apart and then 14 days after the first dose a subject is administered a second series of 5 doses 12 hours apart. As another non-limiting example, a subject is administered two series of doses over a period of 8 weeks where the first series is one dose twice a week for two weeks and the second series of doses is three times a week for 6 weeks.

In certain embodiments, the drug product may be administered at least once after a subject has been administered Betaine. The time between the Betaine administration the drug product administration may be 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, week, 2 weeks, 3 weeks, monthly, 2 months, quarterly, 4 months, 5 months, 6 months, 7 months, 8 months, 9 months, 10 months, 11 months, 12 months, 13 months, 14 months, 15 months, 16 months, 17 months, or 18 months. As a non-limiting example, the drug product may be administered 14 days after the subject was administered Betaine. As another non-limiting example, a subject may be administered the drug product two doses after the subject was administered Betaine. As another non-limiting example, the drug product may be administered 14 or 15 days after Betaine administration.

In certain embodiments, the drug product may be administered in combination with Betaine to a subject. In some embodiments, betaine administration involves self-administration of an over the counter supplement. In some embodiments, betaine administration is prescribed, for example as Cystadane. Betaine administration may include various supplement or drug products, and may comprise betaine HCl, trymethylglycine, betaine anhydrous, or other forms. The combination of betaine and the drug product described herein may be administered at least 1, 2, 3, 4, 5, 6, 7, 8, 9, 10, 11, 12, 13, 14, and 15. In certain embodiments, the drug product may be administered in combination with Betaine more than 15 times. Additional combination therapies that may be administered to a patient include the drug product and at least one treatment to lower tHcy levels, such as very low protein/Met diet and/or vitamins/supplements.

In some embodiments, the dose of the drug product administered to a subject is determined based on such subject characteristics as age, sex, and body weight. For example, the drug product may be provided in a dosage form such as a vial, wherein the amount of the drug product in the dosage form is predetermined based on age of the subject, weight of the subject, and/or sex of the subject. The drug product may be administered in a number of dosage forms over a set time period according to the subject's age, the subject's weight, the subject's sex, and/or other characteristics, in order to adjust the subject's drug product exposure over time. The dosage form of the drug product may be provided at any therapeutically effective amount.

In some embodiments, the dose of the drug product administered to a subject may be between about 0.25 mg/kg and about 10 mg/kg. For example, the dose is one of about 0.33 mg/kg, about 0.66 mg/kg, 1.0 mg/kg, or 1.5 mg/kg. Alternatively, the dose is about 2 mg/kg, about 7 mg/kg, and about 10 mg/kg. For example, the dose may be about 0.5 mg/kg. Alternatively, the therapeutically effective amount is a dosage selected from the range of about 5.0 mg/kg to about 50 mg/kg, and about 10.0 mg/kg to about 25 mg/kg. For example, the dosage is selected from the group consisting of: about 0.25 mg/kg, about 0.33 mg/kg, about 0.66 mg/kg, about 1.00 mg/kg, about 1.10 mg/kg, about 1.20 mg/kg, about 1.30 mg/kg, about 1.40 mg/kg, about 1.50 mg/kg, about 1.60 mg/kg, about 1.70 mg/kg, about 1.80 mg/kg, about 1.90 mg/kg, about 2.00 mg/kg, about 3.00 mg/kg, about 4.00 mg/kg, about 5.00 mg/kg, about 6.00 mg/kg, about 7.00 mg/kg, about 8.00 mg/kg, about 9.00 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, about 25.0 mg/kg, about 26.0 mg/kg, about 27.0 mg/kg, about 28.0 mg/kg, about 29.0 mg/kg, about 30.0 mg/kg, about 31.0 mg/kg, about 32.0 mg/kg, about 33.0 mg/kg, about 34.0 mg/kg, about 35.0 mg/kg, about 36.0 mg/kg, about 37.0 mg/kg, about 38.0 mg/kg, about 39.0 mg/kg, about 40.0 mg/kg, about 41.0 mg/kg, about 42.0 mg/kg, about 43.0 mg/kg, about 44.0 mg/kg, about 45.0 mg/kg, about 46.0 mg/kg, about 47.0 mg/kg, about 48.0 mg/kg, about 49.0 mg/kg, and about 50.0 mg/kg. The doses described herein may be administered once daily or, in some embodiments, twice daily.

In certain embodiments, the drug product is administered to a subject on a methionine-restricted diet. Alternatively, the drug product is administered to a subject that is not on a methionine-restricted diet. In some embodiments, the drug product is administered to a subject having elevated tHcy levels. In some embodiments, the subject is not a genetically-defined HCU patient.

In certain embodiments, the drug product may be co-administered with another therapeutic for treating HCU. As used herein, “co-administered” means the administration of two or more components. These components for co-administration include but are not limited to betaine or Vitamin B6. Co-administration refers to the administration of two or more components simultaneously or with a time lapse between administration such as 1 second, 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, or 3 days. In certain embodiments, the time lapse between administration of two or more components is greater than 3 days.

In certain embodiments, the drug product may be used as a parenteral agent, to be administered to patients chronically via subcutaneous (SC) injection in an initial dosing interval once per week. For example, weekly drug dosing for 6 doses. In certain embodiments, the subject may be within an age range of 18 to 65 years old. In certain embodiments, a subject as young as 16 years of age may be similarly treated.

In certain embodiments, administration occurs over the course of 1 day, 2 days, 3 days, 4 days, 5 days, or 6 days. In certain embodiments, administration occurs over the course of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks.

In some embodiments, subjects having CBS deficiency, including genetically defined HCU patients or non-genetically defined patients having elevated tHcy or CBS deficiency, are administered a varying dose of a treatment as described herein according to the level(s) of one or more metabolic indicator or other indicator or disease severity or progression. For example, a subject may be administered a dosage of 20NHS PEG-CBS within the anticipated therapeutic dose range (e.g., 0.25 to 10 mg/kg twice daily) based on a measurement of the subject's tHcy level or other metabolic indicator level. In this way, subjects can be stratified according to the metabolic indicators of disease severity or progression described herein, and administered a dosage of 20NHS PEG-CBS according to the disease severity or progression.

As a non-limiting example, tHcy level can be measured in a subject having CBS deficiency. The measured level of tHcy can be stratified according to elevated-low, elevated-medium, or elevated-high.

In some embodiments, elevated-low tHcy levels are in the range of about 10 μmol/L to about 50 μmol/L, i.e., about 10 μmol/L, about 15 μmol/L, about 20 μmol/L, about 25 μmol/L, about 30 μmol/L, about 35 μmol/L, about 40 μmol/L, about 45 μmol/L, or about 50 μmol/L.

In some embodiments, elevated-medium tHcy levels are in the range of about 50 μmol/L to about 100 μmol/L, i.e., about 50 μmol/L, about 55 μmol/L, about 60 μmol/L, about 65 μmol/L, about 70 μmol/L, about 75 μmol/L, about 80 μmol/L, about 85 μmol/L, about 90 μmol/L, about 95 μmol/L, or about 100 μmol/L.

In some embodiments, elevated-high tHcy levels are in the range of about 100 μmol/L or higher, for example, about 100 μmol/L to about 1000 μmol/L or about 100 μmol/L to about 500 μmol/L; i.e., about 100 μmol/L, about 110 μmol/L, about 120 μmol/L, about 130 μmol/L, about 140 μmol/L, about 150 μmol/L, about 160 μmol/L, about 170 μmol/L, about 180 μmol/L, about 190 μmol/L, about 200 μmol/L, about 210 μmol/L, about 220 μmol/L, about 230 μmol/L, about 240 μmol/L, about 250 μmol/L, about 260 μmol/L, about 270 μmol/L, about 280 μmol/L, about 290 μmol/L, about 300 μmol/L, about 310 μmol/L, about 320 μmol/L, about 330 μmol/L, about 340 μmol/L, about 350 μmol/L, about 360 μmol/L, about 370 μmol/L, about 380 μmol/L, about 390 μmol/L, about 400 μmol/L, about 410 μmol/L, about 420 μmol/L, about 430 μmol/L, about 440 μmol/L, about 450 μmol/L, about 460 μmol/L, about 470 μmol/L, about 4800 μmol/L, about 490 μmol/L, or about 500 μmol/L.

In some embodiments, a dosage of 20NHS PEG-CBS within the anticipated therapeutic dose range (0.25 to 10 mg/kg) is administered to a subject based on a measurement of the subject's tHcy level as elevated-low, elevated-medium, or elevated-high. For example, a low dose, a medium dose, or a high dose can be administered to the subject, respectively. In some embodiments, a low dose of 20NHS PEG-CBS comprises about 0.25 mg/kg to about 1.0 mg/kg, i.e., about 0.25 mg/kg, about 0.50 mg/kg, about 0.75 mg/kg, or about 1 mg/kg. In some embodiments, a medium dose of 20NHS PEG-CBS comprises about 0.5 mg/kg to about 1.5 mg/kg, i.e., about 0.5 mg/kg, 0.75 mg/kg, 1 mg/kg, 1.25 mg/kg, or about 1.5 mg/kg. In some embodiments, a high dose of 20NHS PEG-CBS comprises about 1 mg/kg to about 2 mg/kg, i.e., about 1 mg/kg, about 1.25 mg/kg, about 1.50 mg/kg, about 1.75 mg/kg, or about 2 mg/kg. In some embodiments, a high dose of 20NHS PEG-CBS comprises a dose higher than about 2 mg/kg, i.e., about 2.25 mg/kg, about 2.50 mg/kg, about 2.75 mg/kg, about 3 mg/kg, about 3.25 mg/kg, about 3.50 mg/kg, about 3.75 mg/kg, about 4 mg/kg, about 4.25 mg/kg, about 4.50 mg/kg, about 4.75 mg/kg, or about 5 mg/kg or higher.

In certain embodiments, drug product is administered as a combination therapy with pyridoxine (also referred to as vitamin B6) and/or an anti-platelet therapy. In some embodiments, vitamin B12 is administered as a combination therapy with pyridoxine and/or an anti-platelet therapy. In some embodiments, folate/folic acid is administered as a combination therapy with pyridoxine and/or an anti-platelet therapy. Accordingly, some embodiments of the methods of treatment provided herein include administering the drug product described herein with one or more of pyridoxine, an anti-platelet therapy, vitamin B12, and/or folate/folic acid.

### VII. Patient Stratification

“Patient stratification,” as used herein, refers to the use of clinical, biochemical, molecular, behavioral, cognitive, or other indicators of disease progression or disease severity in patients having or suspected of having HCU. In some embodiments, stratifying patients according to clinical, biochemical, molecular, behavioral, cognitive, or other indicators of disease progression or disease severity comprises quantitatively or qualitatively recording one or more clinical, biochemical, molecular, behavioral, cognitive, or other indicator of disease progression or disease severity in a patient or patient group and ranking the patient or group on a scale of disease progression or disease severity according to the patient's or group's recorded qualitative or quantitative indicators in comparison with corresponding quantitative or qualitative records or observations from a normal patient population (i.e., a patient population known to not have HCU) and/or a control patient population known to have HCU. Stratifying patients according to the methods described herein can facilitate various aspects of clinical diagnosis, research, and/or treatment of CSBDH. For example, patient strata based on clinical, biochemical, molecular, behavioral, cognitive, or other indicators of disease progression or disease severity described herein can be used to enroll subjects in clinical trials, determine dosage of treatment, determine treatment administration regimes, and/or inform additional treatments or interventions that can be used in combination with the enzyme therapies described herein to alleviate patient symptoms and/or improve patient quality of life. In certain embodiments, individuals eligible for effective enzyme therapy using the drug product described herein include patients having a diagnosis of HCU, based on confirmation of genetic CBS deficient homocystinuria by mutation analysis of CBS gene and a plasma level of tHcy greater than or equal to 80 μM. In certain embodiments, individuals eligible for enrollment in clinical trials of enzyme therapy using the drug product described herein include patients having a diagnosis of HCU, based on confirmation of genetic CBS deficient homocystinuria by mutation analysis of CBS gene and a plasma level of tHcy greater than or equal to 80 μM.

In some embodiments, subjects having CBS deficiency are administered a varying dose of a treatment as described herein according to the level(s) of one or more metabolic indicator. For example, a subject may be administered a dosage of 20NHS PEG-CBS within the anticipated therapeutic dose range (e.g., 0.25 to 10 mg/kg twice daily) based on a measurement of the subject's tHcy level or other metabolic indicator level. In this way, subjects can be stratified according to the metabolic indicators of disease severity or progression described herein, and administered a dosage of 20NHS PEG-CBS according to the disease severity or progression.

As a non-limiting example, tHcy level can be measured in a subject having CBS deficiency. The measured level of tHcy can be stratified according to elevated-low, elevated-medium, or elevated-high.

In some embodiments, elevated-low tHcy levels are in the range of about 10 μmol/L to about 50 μmol/L, i.e., about 10 μmol/L, about 15 μmol/L, about 20 μmol/L, about 25 μmol/L, about 30 μmol/L, about 35 μmol/L, about 40 μmol/L, about 45 μmol/L, or about 50 μmol/L.

In some embodiments, elevated-medium tHcy levels are in the range of about 50 μmol/L to about 100 μmol/L, i.e., about 50 μmol/L, about 55 μmol/L, about 60 μmol/L, about 65 μmol/L, about 70 μmol/L, about 75 μmol/L, about 80 μmol/L, about 85 μmol/L, about 90 μmol/L, about 95 μmol/L, or about 100 μmol/L.

In some embodiments, elevated-high tHcy levels are in the range of about 100 μmol/L or higher, for example, about 100 μmol/L to about 1000 μmol/L or about 100 μmol/L to about 500 μmol/L; i.e., about 100 μmol/L, about 110 μmol/L, about 120 μmol/L, about 130 μmol/L, about 140 μmol/L, about 150 μmol/L, about 160 μmol/L, about 170 μmol/L, about 180 μmol/L, about 190 μmol/L, about 200 μmol/L, about 210 μmol/L, about 220 μmol/L, about 230 μmol/L, about 240 μmol/L, about 250 μmol/L, about 260 μmol/L, about 270 μmol/L, about 280 μmol/L, about 290 μmol/L, about 300 μmol/L, about 310 μmol/L, about 320 μmol/L, about 330 μmol/L, about 340 μmol/L, about 350 μmol/L, about 360 μmol/L, about 370 μmol/L, about 380 μmol/L, about 390 μmol/L, about 400 μmol/L, about 410 μmol/L, about 420 μmol/L, about 430 μmol/L, about 440 μmol/L, about 450 μmol/L, about 460 μmol/L, about 470 μmol/L, about 4800 μmol/L, about 490 μmol/L, or about 500 μmol/L.

In some embodiments, a dosage of 20NHS PEG-CBS within the anticipated therapeutic dose range (e.g., 0.25 to 10 mg/kg twice daily) is administered to a subject based on a measurement of the subject's tHcy level as elevated-low, elevated-medium, or elevated-high. For example, a low dose, a medium dose, or a high dose can be administered to the subject, respectively. In some embodiments, a low dose of 20NHS PEG-CBS comprises about 0.25 mg/kg to about 1.0 mg/kg, i.e., about 0.25 mg/kg, about 0.50 mg/kg, about 0.75 mg/kg, or about 1 mg/kg. In some embodiments, a medium dose of 20NHS PEG-CBS comprises about 0.5 mg/kg to about 1.5 mg/kg, i.e., about 0.5 mg/kg, 0.75 mg/kg, 1 mg/kg, 1.25 mg/kg, or about 1.5 mg/kg. In some embodiments, a high dose of 20NHS PEG-CBS comprises about 1 mg/kg to about 2 mg/kg, i.e., about 1 mg/kg, about 1.25 mg/kg, about 1.50 mg/kg, about 1.75 mg/kg, or about 2 mg/kg. In some embodiments, a high dose of 20NHS PEG-CBS comprises a dose higher than about 2 mg/kg, i.e., about 2.25 mg/kg, about 2.50 mg/kg, about 2.75 mg/kg, about 3 mg/kg, about 3.25 mg/kg, about 3.50 mg/kg, about 3.75 mg/kg, about 4 mg/kg, about 4.25 mg/kg, about 4.50 mg/kg, about 4.75 mg/kg, or about 5 mg/kg or higher.

A. Clinical Presentation

According to the Guidelines for the Diagnosis and Management of HCU, the disease should be suspected in children presenting with severe or rapidly progressing myopathy, lens dislocation and/or developmental delays (see Morris et al. Guidelines for the diagnosis and management of cystathionine beta-synthase deficiency. J Inherit Metab Dis 2017; 40:49-74, which is hereby incorporated by reference in its entirety). Tests are also warranted in adults presenting with thromboembolism and/or lens dislocation but no other symptoms and in those with multi-system disease, including ocular, connective tissue, neuro-psychiatric and vascular complications (see Mudd et al. Am J Hum Genet 1985; 37:1-31; Morris et al. Guidelines for the diagnosis and management of cystathionine beta-synthase deficiency. J Inherit Metab Dis 2017; 40:49-74; Kelly et al. Neurology 2003; 60:275-279, each of which is hereby incorporated by reference in its entirety).

B. Biochemical Analysis

1. Total Homocysteine Levels

Levels of plasma tHcy are determined using the sum of all free and bound homocysteine species after treating the plasma with a reducing agent. In healthy individuals with stable dietary habits, tHcy levels remain relatively constant over time (see Refsum et al. Clin Chem 2004; 50:3-32; McKinley et al. Clin Chem 2001; 47:1430-1436; each of which is hereby incorporated by reference in its entirety). However, consumption of a protein-rich meal can increase tHcy levels by approximately 10% over a period of several hours (see Verhoef et al. Am J Clin Nutr 2005; 82:553-558, which is hereby incorporated by reference in its entirety). A study in individuals with hyperhomocysteinemia (tHcy>40 μmol/L) found that intra-individual tHcy levels varied up to 25% over a period of 4 to 8 months. However, no information was provided on the variability of the diets, assay methods, and times of sampling, hindering the ability to interpret the data (see Refsum H, Smith A D, Ueland P M et al. Facts and recommendations about total homocysteine determinations: an expert opinion. Clin Chem 2004; 50:3-32, which is hereby incorporated by reference in its entirety).

In populations without folate supplementation, corresponding upper reference limits are approximately 15 and 20 μmol/L, respectively. To support a diagnosis of CBS in a neonate, tHcy in plasma is expected to be between 50 to greater than 100 μmol/L and Met in plasma is expected to be between 200 to 1500 μmol/L (i.e., 3-23 mg/dL) (see Sacharow et al. Homocystinuria Caused by Cystathionine Beta-Synthase Deficiency. GeneReviews 2017, which is hereby incorporated by reference in its entirety).To support a diagnosis of CBS in an untreated older individual, tHcy in plasma is expected to be greater than 100 μmol/L and Met in plasma is expected to be greater than 50 μmol/L (i.e., greater than 0.7 mg/dL). A control neonate or older individual would be expected to have tHcy in plasma less than 15 μmol/L and Met between 10 to 40 μmol/L (0.2-0.6 mg/dL).

In some embodiments, subjects having elevated tHcy and/or CBS deficiency are administered a varying dose of a treatment as described herein according to the level(s) of tHcy. For example, a subject may be administered a dosage of 20NHS PEG-CBS within the anticipated therapeutic dose range (0.25 to 10 mg/kg) based on a measurement of the subject's tHcy level. In this way, subjects can be stratified according to the metabolic indicators of disease severity or progression described herein, and administered a dosage of 20NHS PEG-CBS according to the disease severity or progression.

As a non-limiting example, tHcy level can be measured in a subject having CBS deficiency. The measured level of tHcy can be stratified according to elevated-low, elevated-medium, or elevated-high.

In some embodiments, elevated-low tHcy levels are in the range of about 10 μmol/L to about 50 μmol/L, i.e., about 10 μmol/L, about 15 μmol/L, about 20 μmol/L, about 25 μmol/L, about 30 μmol/L, about 35 μmol/L, about 40 μmol/L, about 45 μmol/L, or about 50 μmol/L.

In some embodiments, elevated-medium tHcy levels are in the range of about 50 μmol/L to about 100 μmol/L, i.e., about 50 μmol/L, about 55 μmol/L, about 60 μmol/L, about 65 μmol/L, about 70 μmol/L, about 75 μmol/L, about 80 μmol/L, about 85 μmol/L, about 90 μmol/L, about 95 μmol/L, or about 100 μmol/L.

In some embodiments, elevated-high tHcy levels are in the range of about 100 μmol/L or higher, for example, about 100 μmol/L to about 1000 μmol/L or about 100 μmol/L to about 500 μmol/L; i.e., about 100 μmol/L, about 110 μmol/L, about 120 μmol/L, about 130 μmol/L, about 140 μmol/L, about 150 μmol/L, about 160 μmol/L, about 170 μmol/L, about 180 μmol/L, about 190 μmol/L, about 200 μmol/L, about 210 μmol/L, about 220 μmol/L, about 230 μmol/L, about 240 μmol/L, about 250 μmol/L, about 260 μmol/L, about 270 μmol/L, about 280 μmol/L, about 290 μmol/L, about 300 μmol/L, about 310 μmol/L, about 320 μmol/L, about 330 μmol/L, about 340 μmol/L, about 350 μmol/L, about 360 μmol/L, about 370 μmol/L, about 380 μmol/L, about 390 μmol/L, about 400 μmol/L, about 410 μmol/L, about 420 μmol/L, about 430 μmol/L, about 440 μmol/L, about 450 μmol/L, about 460 μmol/L, about 470 μmol/L, about 4800 μmol/L, about 490 μmol/L, or about 500 μmol/L.

In some embodiments, a dosage of 20NHS PEG-CBS within the anticipated therapeutic dose range (0.25 to 10 mg/kg) is administered to a subject based on a measurement of the subject's tHcy level as elevated-low, elevated-medium, or elevated-high. For example, a low dose, a medium dose, or a high dose can be administered to the subject, respectively. In some embodiments, a low dose of 20NHS PEG-CBS comprises about 0.25 mg/kg to about 1.0 mg/kg, i.e., about 0.25 mg/kg, about 0.50 mg/kg, about 0.75 mg/kg, or about 1 mg/kg. In some embodiments, a medium dose of 20NHS PEG-CBS comprises about 0.5 mg/kg to about 1.5 mg/kg, i.e., about 0.5 mg/kg, 0.75 mg/kg, 1 mg/kg, 1.25 mg/kg, or about 1.5 mg/kg. In some embodiments, a high dose of 20NHS PEG-CBS comprises about 1 mg/kg to about 2 mg/kg, i.e., about 1 mg/kg, about 1.25 mg/kg, about 1.50 mg/kg, about 1.75 mg/kg, or about 2 mg/kg. In some embodiments, a high dose of 20NHS PEG-CBS comprises a dose higher than about 2 mg/kg, i.e., about 2.25 mg/kg, about 2.50 mg/kg, about 2.75 mg/kg, about 3 mg/kg, about 3.25 mg/kg, about 3.50 mg/kg, about 3.75 mg/kg, about 4 mg/kg, about 4.25 mg/kg, about 4.50 mg/kg, about 4.75 mg/kg, or about 5 mg/kg or higher.

2. Methionine Levels

High to high-normal Met levels (reference ranges are typically 40 to 45 and 12 to 15 μmol/L, respectively) in combination with low to low-normal Cth levels (reference ranges 0.05 to 0.08 and 0.35 to 0.5 μmol/L, respectively) may be useful for distinguishing HCU from HCU caused by genetic and nutritional disorders of Hcy remethylation (see Morris et al. J Inherit Metab Dis 2017; 40:49-74; Stabler et al. JIMD Rep 2013; 11:149-163; Bartl et al. Clin Chim Acta 2014; 437:211-217, each of which is hereby incorporated by reference in its entirety). Another useful test determines Cth production from Hcy and serine in cultured fibroblasts, using radioactive or deuterium labeled substrates (see Morris et al. J Inherit Metab Dis 2017; 40:49-74; Kraus JP. Methods Enzymol 1987; 143:388-394; Smith et al. J Chromatogr B Analyt Technol Biomed Life Sci 2012; 911:186-191, each of which is hereby incorporated by reference in its entirety). However, enzyme analysis cannot always distinguish between pyridoxine responsive and non-responsive individuals and the enzymatic activity may be normal in mild cases (see Alcaide et al. Clin Chim Acta 2015; 438:261-265, which is hereby incorporated by reference in its entirety). More recently, rapid stable isotope assays measuring activity of CBS released from organs into plasma showed 100% sensitivity in pyridoxine non-responsive patients, but only 86% sensitivity in pyridoxine responders (see Alcaide et al. Clin Chim Acta 2015; 438:261-265; Krijt et al. J Inherit Metab Dis 2011; 34:49-55, both of which are hereby incorporated by reference in their entirety).

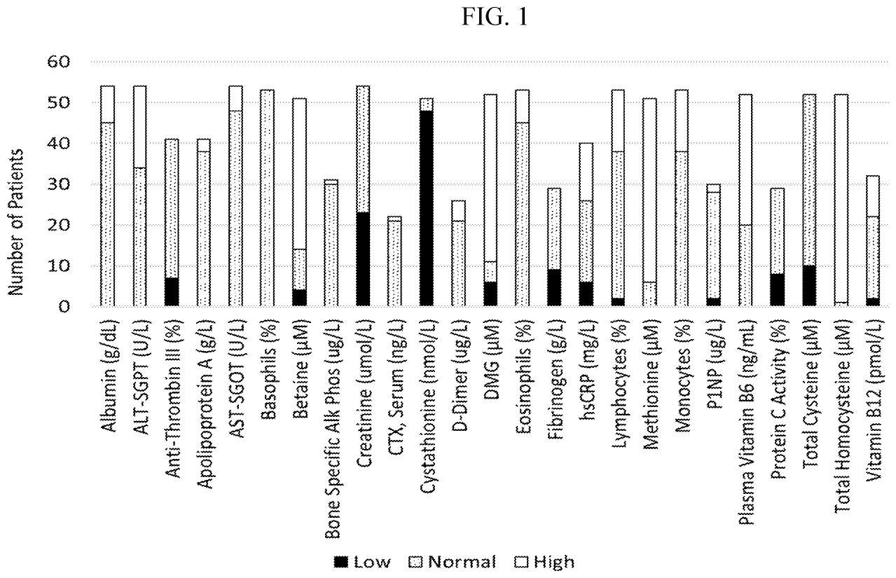

Methionine levels ≥600 and in some cases greater than ≥1000 as well as dimethylglycine (DMG) levels above the upper limit of normal (ULN), are observed in significant numbers even in patients undergoing treatment using a natural protein-restricted diet and/or a Met-free L-amino acid mixture and supplements, including betaine, as discussed in Example 4-5 and demonstrated in Table 5 and FIG. 1. These data demonstrate that current diet and therapeutic interventions are poorly effective. In particular, betaine is not in itself adequate treatment for HCU, contrary to common belief. Further research is needed to understand the correlations between patient response to conventional treatments and elevated methionine, DMG, and other biochemical indicators. In some embodiments of the methods described herein, measuring and tracking levels of various biochemical indicators can be useful in identifying and stratifying HCU subjects in terms of disease severity and patient-response to treatments.

In some embodiments, subjects having CBS deficiency are administered a varying dose of a treatment as described herein according to the level(s) of methionine. For example, a subject may be administered a dosage of 20NHS PEG-CBS within the anticipated therapeutic dose range (0.25 to 10 mg/kg) based on a measurement of the subject's methionine level. In this way, subjects can be stratified according to the metabolic indicators of disease severity or progression described herein, and administered a dosage of 20NHS PEG-CBS according to the disease severity or progression.

As a non-limiting example, tHcy level can be measured in a subject having CBS deficiency. The measured level of tHcy can be stratified according to elevated-low or elevated-high. In some embodiments, an elevated-low methionine level is about 10 μmol/L to about 100 μmol/L, and an elevated high methionine level is about 100 μmol/L to about 500 μmol/L, or higher than about 150 μmol/L.

3. Creatinine Levels

Creatinine levels below the lower limit of normal (LLN) can be used to identify and stratify HCU in pediatric and adult patients, including stratifying based on patient response to methionine restricted diet and/or betaine and/or vitamin supplementation. Decreased creatinine levels can be due to low muscle mass secondary to protein restriction in subjects on protein-restricted diets. Therefore, creatinine levels below the LLN can be used to monitor and stratify subjects according to restricted diet and/or restricted diet compliance.

4. C-Reactive Protein Levels

High Sensitivity C-Reactive Protein (hsCRP) levels above the ULN can be used to identify and stratify HCU in patients, including stratifying based on patient response to methionine restricted diet and/or betaine and/or vitamin supplementation.

Low Protein C activity levels and/or low fibrinogen levels can be used to identify and stratify HCU in patients, including stratifying based on patient response to methionine restricted diet and/or betaine and/or vitamin supplementation.

5. Other Metabolic Indicators

More refined biochemical analysis using a number of biochemical indicators individually or in combination can be used to stratify patients for diagnosis or treatment of HCU (see Example 5, Table 5, FIG. 1). The biochemical markers can alternatively be used to monitor patient response to traditional non-ET treatments. ET treatment methods as described herein can, in some cases, alleviate elevation or reduction outside normal ranges of these biochemical markers in subjects on methionine restricted diets or in subjects taking vitamin or betaine supplements. In addition to elevated tHcy and Met and low cystathionine and total cysteine levels, the following biochemical indicators can help to identify and stratify HCU in subjects, including based on patient response to restricted diet and/or betaine and vitamin supplement treatment methods.

In some embodiments, methionine levels, DMG levels, ALT levels, creatinine levels, hsCRP levels, and/or Protein C activity levels can be used individually or in combination to identify and stratify HCU in patients, including stratifying based on patient response to methionine restricted diet and/or betaine and/or vitamin supplementation. Use of multiple of these biochemical indicators can increase sensitivity and accuracy of diagnosis and stratification. These biochemical indicators can further be combined with biochemical indicators known to be within normal ranges in HCU patients. For example, normal levels of aspartate aminotransferase (AST), anti-thrombin III, and apolipoprotein A can be used to further identify and stratify HCU patients.

6. Adjustment of CBS Treatment According to Metabolic Indicators

In some embodiments, the administration of a treatment for CBS deficiency as described herein can be dose-adjusted according the levels of one or more metabolic indicators of disease severity or progression provided herein. For example, if a measurement or determination of one or more metabolic indicators of disease progression or severity indicates severe or advanced progression of CBS deficiency a CBS treatment such as 20NHS PEG-CBS can be administered at a low dose, a medium dose, or a high dose. In some embodiments, a low dose of 20NHS PEG-CBS comprises about 0.25 mg/kg to about 1.0 mg/kg, i.e., about 0.25 mg/kg, about 0.50 mg/kg, about 0.75 mg/kg, or about 1 mg/kg. In some embodiments, a medium dose of 20NHS PEG-CBS comprises about 0.5 mg/kg to about 1.5 mg/kg, i.e., about 0.5 mg/kg, 0.75 mg/kg, 1 mg/kg, 1.25 mg/kg, or about 1.5 mg/kg. In some embodiments, a high dose of 20NHS PEG-CBS comprises about 1 mg/kg to about 2 mg/kg, i.e., about 1 mg/kg, about 1.25 mg/kg, about 1.50 mg/kg, about 1.75 mg/kg, or about 2 mg/kg. In some embodiments, a high dose of 20NHS PEG-CBS comprises a dose higher than about 2 mg/kg, i.e., about 2.25 mg/kg, about 2.50 mg/kg, about 2.75 mg/kg, about 3 mg/kg, about 3.25 mg/kg, about 3.50 mg/kg, about 3.75 mg/kg, about 4 mg/kg, about 4.25 mg/kg, about 4.50 mg/kg, about 4.75 mg/kg, or about 5 mg/kg or higher.

C. Molecular Diagnosis

Molecular genetic testing can be performed by either single-gene testing or using a multi-gene panel (see Yap S. Homocystinuria due to cystathionine beta-synthase deficiency. Orphanet Encyclopaedia [serial online] 2005; Sacharow S J, Picker J D, Levy H L. Homocystinuria Caused by Cystathionine Beta-Synthase Deficiency. GeneReviews 2017; Morris et al. J Inherit Metab Dis 2017; 40:49-74; Katsanis et al. Nat Rev Genet 2013; 14:415-426; each of which is hereby incorporated by reference in its entirety). Individuals with high risk of having a particular CBS mutation should be screened using targeted single-gene testing. However, this is only useful in select populations with a common CBS mutation, (e.g. 93% of individuals with HCU from Qatar carry the p.Arg336Cys; c.1006C>T mutation) and in individuals from families with a known pathogenic variant. In other patients, the CBS gene can be sequenced, and gene-targeted deletion/duplication analysis performed only if one or no pathogenic variant is found. Table 3 provides a non-limiting list of genetic mutations that can be used identify and stratify HCU in patients.

Alternatively, simultaneous molecular testing of multiple genes can be performed using a multiple gene panel. Methods used may include sequence analysis, deletion/duplication analysis and other non-sequencing-based tests (see Morris et al. J Inherit Metab Dis 2017; 40:49-74, which is hereby incorporated by reference in its entirety). Generally, molecular genetic testing is reserved for high-risk populations with a limited number of prevalent mutations (see Morris et al. J Inherit Metab Dis 2017; 40:49-74; Huemer et al. J Inherit Metab Dis 2015; 38:1007-101, each of which is hereby incorporated by reference in its entirety).

D. Pyridoxine Responsiveness Tests

Pyridoxine-responsiveness tests are used in the clinic to determine whether pyridoxine supplementation should be prescribed for patients with HCU. Because different treatment centers have defined pyridoxine responsiveness differently (see Morris et al. J Inherit Metab Dis 2017; 40:49-74, which is hereby incorporated by reference in its entirety), classification of patients by tHcy levels, rather than by their pyridoxine responsiveness, is more rigorous. Pyridoxine-responsiveness is not a measure of metabolic control but rather an indication that there remains some residual CBS activity.

E. Newborn Screening (NBS)

In general, NBS tests for HCU deficiency are carried out by analyzing dried blood spots to determine Met levels. Alternately, assessment of tHcy levels rather than Met in dried blood spots for NBS is available in a few centers worldwide. It is employed as a second-tier test to reduce the false-positive rates of NBS in individuals with high Met levels (see Turgeon et al. Clin Chem 2010; 56:1686-1695, which is hereby incorporated by reference in its entirety) and is not used to improve sensitivity or reduce false-negative rates.

F. Cognitive Function

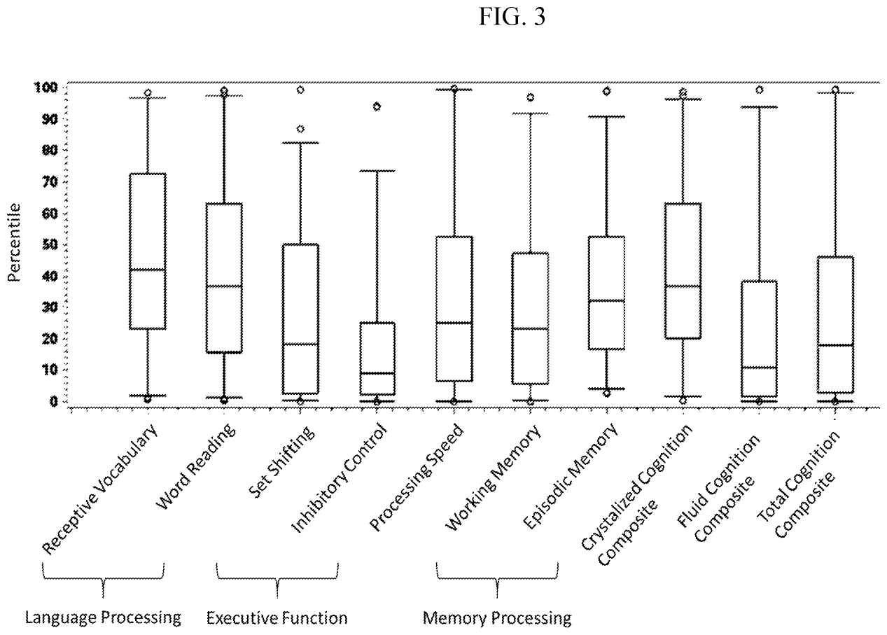

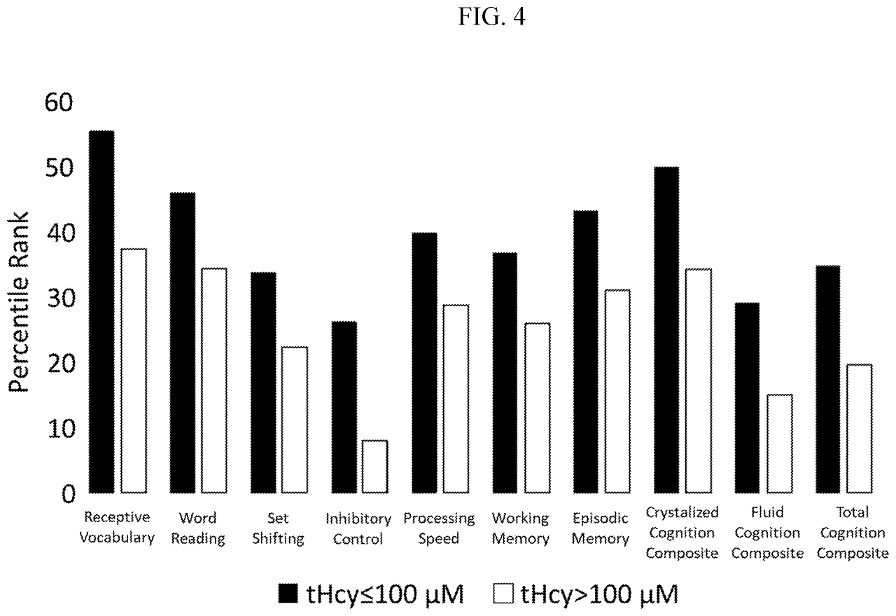

Patients with lower plasma tHcy levels perform better on measures of executive functioning. The NIH Toolbox or other assessments of neurobehavioral or cognitive function can be used to assess and stratify patients according to cognitive functioning over time, including tracking response to intervention. Correlations between cognition and tHcy levels can further be used to stratify patients (see, e.g., Table 9 and FIG. 3 and FIG. 4). Reduced tHcy levels in a subject following ET as described herein can be used as an indicator that the subject will have improved outcome for neurobehavioral or cognitive intervention. Elevated tHcy levels, on the other hand, can indicate that a subject is in need of or will benefit from neurobehavioral or cognitive intervention in combination with ET as described herein. For example, tHcy levels >50 μM, >60 μM, >70 μM, >80 μM, >90 μM, or >100 μM can be used as a threshold for measuring and/or tracking neurobehavioral and cognitive function and/or as an indicator of patient susceptibility to or need for neurobehavioral or cognitive treatment or intervention in patients having or suspected of having HCU. In some embodiments, neurobehavioral or cognitive treatment or intervention includes supports similar to those for other executive functioning problems, such as ADHD, including, for example, behavioral therapies such as behavioral parent training (BPT) and behavioral classroom management (BCM) (see, e.g., Pelham Jr, William E., and Gregory A. Fabiano. “Evidence-based psychosocial treatments for attention-deficit/hyperactivity disorder.” Journal of Clinical Child & Adolescent Psychology 37.1 (2008): 184-214; and Pfiffner, Linda J., and Lauren M. Haack. “Behavior management for school-aged children with ADHD.” Child and Adolescent Psychiatric Clinics 23.4 (2014): 731-746, the contents of each which are incorporated by reference herein in their entirety).

G. Non-Genetically-Defined Patient Stratification

The compositions and methods described here are useful in treating or alleviating complications or conditions associated with elevated tHcy levels, including in non-genetically-defined subjects or populations having elevated tHcy levels.

Using the Broad Institute GnomAD database to conservatively screen for the frequency of CBS mutations, it is estimated that thousands of patients are prevalent in the US with loss-of-function and/or missense mutation variants of CBS. Some estimates provide for over 4,000 individuals with loss-of-function and/or missense mutation variants of CBS, while other estimates provide for over 14,000 individuals with loss-of-function and/or missense mutation variants of CBS.

tHcy testing is common and inexpensive. An estimated 5 million patients per year have their tHcy tested. 31,000 to 35,000 patients demonstrate tHcy levels more than 2 standard deviations above the mean. Many patients with elevated tHcy have a history of diagnoses that are also associated with classical HCU, yet fewer than 10% have a recorded diagnosis of HCU or a sulfur amino acid metabolism disorder. Many of these patients may have non-genetically-defined elevated tHcy levels.

Elevated tHcy levels, as used herein, refers to tHcy levels (i.e., total plasma homocysteine levels) higher than the normal mean value expected for a subject based on the subjects age, sex, diet, or other factors. In some embodiments, subjects having elevated tHcy levels have higher than about 4 μmol/L, higher than about 5 μmol/L, higher than about 6 μmol/L, higher than about 7 μmol/L, higher than about 8 μmol/L, higher than about 9 μmol/L, higher than about 10 μmol/L, higher than about 11 μmol/L, higher than about 12 μmol/L, higher than about 13 μmol/L, higher than about 14 μmol/L, higher than about 15 μmol/L, higher than about 16 μmol/L, higher than about 17 μmol/L, higher than about 18 μmol/L, higher than about 19 μmol/L, higher than about 20 μmol/L, higher than about 21 μmol/L, higher than about 22 μmol/L, higher than about 23 μmol/L, higher than about 24 μmol/L, higher than about 25 μmol/L, higher than about 26 μmol/L, higher than about 27 μmol/L, higher than about 28 μmol/L, higher than about 29 μmol/L, higher than about 30 μmol/L. In some embodiments, elevated tHcy levels can be as high as 100 μmol/L. In some embodiments, elevated tHcy levels can be higher than 100 μmol/L. Thus, some embodiments of the present disclosure provide for elevated tHcy levels in a subject as being between about 4 μmol/L up to about 100 μmol/L, or higher.

Accordingly, some embodiments of the described methods and compositions comprise stratifying a patient population based on an elevated tHcy level, and selecting subjects within the population for treatment of elevated tHcy levels, including treating and alleviating conditions and complications associated with elevated tHcy levels. In some embodiments, treating subjects having elevated tHcy levels alleviates related conditions and complications, such as cognitive or skeletal abnormalities, independent of any underlying pathology, e.g., CBS deficiency or HCU.

### VIII. Phenotypic Outcomes

Retrospective studies show a proportional relationship between tHcy levels and outcomes. Patients with the highest tHcy levels (treated or untreated) present with more severe symptoms earlier in life, whereas patients with lower tHcy levels present with fewer symptoms and progress less rapidly (see Yap S. Homocystinuria due to cystathionine beta-synthase deficiency. Orphanet Encyclopaedia [serial online] 2005; Mudd et al. Am J Hum Genet 1985; 37:1-31, each of which is hereby incorporated by reference in its entirety). Individuals with elevated tHcy levels typically present with failure to thrive, thromboembolism, severe myopia with subsequent dislocation of the optic lens, osteoporosis-type fractures, a Marfanoid habitus (in particular elongation of the long bones) and/or psychiatric abnormalities such as learning difficulties (see Yap S. Homocystinuria due to cystathionine beta-synthase deficiency. Orphanet Encyclopaedia [serial online] 2005; Morris et al. J Inherit Metab Dis 2017; 40:49-74; NORD, Kraus J P. Homocystinuria due to cystathionine beta-synthase deficiency. NORD [serial online] 2017, each of which are hereby incorporated by reference in its entirety). Reflecting the spectrum of CBS deficiency, some patients have a severe childhood-onset multisystemic disease, whereas those with less severely elevated Hcy may remain undiagnosed into adulthood (see Morris et al. J Inherit Metab Dis 2017; 40:49-74, which is hereby incorporated by reference in its entirety). Life expectancy is markedly reduced in patients with severely elevated Hcy levels, though even patients with moderately elevated tHcy levels suffer from multiple negative clinical outcomes.

Significant evidence has been observed to indicate the causal effect of elevated tHcy levels and negative clinical outcomes in the four systems commonly affected in HCU patients (ocular, skeletal, cardiovascular, and neurologic). In the ocular system, frequently observed phenotypic outcomes include: ectopia lentis, iridodonesis, myopia, and less frequently observed phenotypic outcomes include: glaucoma, optic atrophy, retinal degeneration, retinal detachment, cataracts, and corneal abnormalities. In the skeletal system, frequently observed phenotypic outcomes include: osteoporosis, biconcave vertebrae, scoliosis, Increased length of long bones, Irregular widened metaphysis, metaphyseal spicules, abnormal size/shape of epiphyses, growth arrest lines, pes cavus, and high-arched palate, and less frequently observed phenotypic outcomes include: arachnodactyly, enlarged carpal bones, abnormal bone age, pectus carinatum/excavatum, genu valgum, kyphosis, and short fourth metacarpal. In the vascular system, frequently observed phenotypic outcomes include: vascular occlusions, malar flush, and livedo reticularis. In the central nervous system, frequently observed phenotypic outcomes include: cognitive symptoms, psychiatric disturbances, and extrapyramidal signs, and less frequently observed phenotypic outcomes include: seizures and abnormal electroencephalogram. In additional body systems, the following phenotypic outcomes are frequently observed fair, brittle hair, thin skin, fatty changes in liver, inguinal hernia, myopathy, endocrine abnormalities, reduced clotting factors, and spontaneous bowel perforation.

A strong relationship has been observed between mildly elevated levels of tHcy and negative outcomes, but data has also indicated that lowering tHcy levels positively impacts clinical manifestations. The literature on HCU suffers from the rarity of the disease and subsequent smaller studies but benefits from the magnification of clinical outcomes in a population with severely elevated Hcy levels. Conversely, studies in the broader population benefit from large sample sizes but smaller elevations in tHcy levels. Taken together these studies consistently demonstrate that elevated tHcy levels are strongly predictive of negative clinical outcomes and that pharmacological intervention to reduce those levels is beneficial.

A potential outcome from treatment of HCU with the drug product described herein is to lower the plasma tHcy concentration to the lowest possible levels while maintaining a more relaxed diet, including higher concentrations of Met than provided in other therapies for HCU and other essential amino acids. In infants and children with HCU, the priority is to prevent complications associated with HCU and to ensure proper growth and development of normal intelligence (see Morris et al. J Inherit Metab Dis. 2017 January; 40(1):49-74, which is hereby incorporated by reference in its entirety). In patients diagnosed later in life, the priority may be to prevent life-threatening thromboembolism and to minimize progression of already established complications. To address these goals, the biochemical abnormalities associated with HCU may be improved and, if possible, normalized (see Morris et al. J Inherit Metab Dis. 2017 January; 40(1):49-74, which is hereby incorporated by reference in its entirety). A survey comparing dietetic management practices for patients with HCU across 29 centers in 8 European countries, found that there was little consensus in treatment centers about target ranges for plasma tHcy levels, with the median recommended target of less than 55 μM and within a range of less than 20 to 100 μM among the 29 centers (see Adam et al. Mol Genet Metab. 2013 December; 110(4):454-9, which is hereby incorporated by reference in its entirety).

The cut-off value of greater than or equal to 80 μM for tHcy levels for eligibility for treatment was chosen herein to avoid excluding patients with prior plasma tHcy levels of about 100 μM given a within-person variability of 25% in plasma tHcy levels tested several months apart (see Refsum et al. Clin Chem 2004; 50:3-32; Guttormsen et al., J Clin Invest. 1996, 98(9):2174-83; each of which is hereby incorporated by reference in its entirety) to provide levels high enough to detect clinically significant reductions in a small number of patients. The within-person variability of 25% in plasma tHcy levels tested several months apart (see Refsum et al. Clin Chem 2004; 50:3-32; Guttormsen et al., J Clin Invest. 1996, 98(9):2174-83; each of which is hereby incorporated by reference in its entirety) may be partially due to changes in diet, medications, or supplements in HCU patients over time.

If untreated, the prognosis for patients with pyridoxine-unresponsive HCU is bleak (see Morris et al. J Inherit Metab Dis 2017; 40:49-74, which is hereby incorporated by reference in its entirety). In 1985, an international retrospective study documenting the natural history of HCU in 629 patients, by time-to-event analyses before treatment, showed that 70% of patients experienced lens dislocation by the age of 10 years, with 85% developing symptoms by 12 years (see Mudd et al. Am J Hum Genet 1985; 37:1-31; Mudd et al., Skovby F. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327, both of which are hereby incorporated by reference in its entirety). Overall, 50% of affected individuals had radiographically detected spinal osteoporosis by the age of 15 years and 23% of pyridoxine non-responsive patients (4% of responsive patients) died by the age of 30 years (see Mudd et al. Am J Hum Genet 1985; 37:1-31, which is hereby incorporated by reference in its entirety).

Taken in its entirety, the available evidence indicates that the current approaches to treatment of HCU, including restrictive diet and use of dietary supplements, are ineffective in halting progression of the disease progression in most patients. Consequently, there is a substantial unmet medical need to identify well tolerated therapies that will improve or normalize the metabolic abnormalities of HCU and slow or halt progression of the clinical manifestations of the disease.

A causal effect between increased Hcy levels and key clinical outcomes associated with HCU, including ocular complications (in particular, lens dislocation), skeletal outcomes (in particular, osteoporosis), vascular events (in particular, stroke and small vessel disease) and various CNS outcomes (in particular, cognitive function), has been observed. The relationship between elevated tHcy levels and negative clinical outcomes and conversely, by reduced Hcy levels and improved clinical outcomes, is further strengthened by multiple studies in the general population. Overall, these clinical findings highlight the need for early HCU diagnosis and prompt treatment to decrease Hcy levels to as close as possible to normal.

Although no studies have been published on the Quality of Life (QoL) of HCU patients, unpublished reports indicate that patients and their caregivers suffer from the psychosocial effects of following and managing a highly restricted and socially isolating diet and are extremely anxious about the long-term medical consequences of the disease. Not surprisingly, patients yearn for the ability to relax their diets without compromising their long-term prospects.

The strong relationship between tHcy levels and key clinical outcomes in patients with HCU shows that the change in tHcy levels is a reliable surrogate marker for a combination of clinical endpoints in HCU. Changes in tHcy levels are therefore useful for (i) monitoring patient progress in the clinic and (ii) predicting the clinical benefits of new treatments in a clinical trial and (iii) predicting the efficacy of a therapy.

tHcy levels also provide a reliable surrogate for selection of subjects having non-genetically defined CBS deficiency. According to the methods described herein, subjects having elevated tHcy and/or having CBS deficiency can be treated using the PEGylated htCBS C15S drug product described herein to treat or ameliorate symptoms associated with elevated tHcy, including ocular, skeletal, cardiovascular, and neurologic symptoms of elevated tHcy.

For example, the drug product normalizes or increases femoral artery flexibility in a subject compared to before administration of the drug product to the subject. For example, I278T mice have significantly lower femoral artery flexibility compared to wildtype mice. A Met-restricted diet may, in fact, result in a smaller femoral artery diameter in I278T mice compared to a regular diet in both mice treated with the drug products and those that are not.

Studies previously conducted in 3 murine models of the disease have demonstrated that htCBS C15S is effective after systemic administration as described in WO 2017/083327, which is hereby incorporated by reference in its entirety. These studies showed an up to 90% decrease in extracellular Hcy plasma and intracellular Hcy levels in tissue, such as brain. Administration of the drug product was observed to result in a concentration gradient, with flux of Hcy from higher concentrations in the intracellular space to the lower concentrations in the extracellular space where the drug product can further process it. The extracellular PEGylated htCBS C15S serves as a Hcy “sink.” In summary, drug product restored control of the Met metabolism pathway in animal models of HCU.

These studies have shown also that SC dosing of PEGylated htCBS C15S in murine models of HCU corrected metabolite levels, including elevation in Cth levels and normalization of Cys levels. In addition, PEGylated htCBS C15S positively affected the phenotypic expression of the disease in mice, including facial alopecia, liver histology, osteoporosis, body composition, diabetic retinopathy (possibly secondary to renal disease) and macular and optic atrophy due to retinal vascular occlusion or non-arteritic ischemic optic neuropathy, cytokines, and lipid levels. PEGylated htCBS C15S also rescued CBS knockout (KO) mice from early death (see Looker et al. Diabetologia 2003; 46:766-772; Pusparajah et al. Front Physiol 2016; 7:200; Gerth et al. J AAPOS 2008; 12:591-596; Stanger et al. Clin Chem Lab Med 2005; 43:1020-1025; Cahill et al. Am J Ophthalmol 2003; 136:1136-1150; Minniti et al. Eur J Ophthalmol 2014; 24:735-743; each of which is hereby incorporated by reference in its entirety). PEGylated htCBS C15S was also observed to be well tolerated with no toxicological effects noted with chronic dosing in animal models of the disease.

PEGylated htCBS C15S acts in the extracellular space and is anticipated to lower tHcy plasma concentrations regardless of the patients' genetics, concurrent therapy, or baseline tHcy level. Thus, the eligible population for the study should include both pyridoxine responsive and nonresponsive patients.

In healthy individuals, tHcy levels are in the range of approximately 5 to 15 μM (OECD Environmental Health and Safety Publications. Series on Principles of Good Laboratory Practice and Compliance Monitoring. No. 1 ENV/MC/CHEM(98)17 “Principles of Good Laboratory Practice (as revised in 1997), which is hereby incorporated by reference in its entirety), 98% of which is in the form of disulfides or is protein bound. Only 2% of the tHcy exists as a non-bound, free, reduced aminothiol that can serve as a substrate for the enzyme (see EMA: Guideline on bioanalytical method validation, EMEA/CHMP/EWP/192217/2009, ev. 1, 21 Jul. 2011; ATL-15-1419 Atlanbio Study Report “LC-MS/MS determination of cystathionine-D4 as product of the cystathionine β-synthase activity in monkey plasma samples collected during the study 529736”; which are both hereby incorporated by reference in their entireties). HCU patients on the other hand, not only present with plasma levels that may reach 400 μM and more, but also present with a dramatically altered balance, with free homocysteine reaching 10-25% of the tHcy values.

In mouse models, administration of PEGylated htCBS C15S resulted in up to 90% reduction in tHcy levels. Thus, the initial levels of free homocysteine available for the enzyme (10%-25% of the total) cannot solely account for the significant decrease in tHcy levels that were recorded, and additional pools must become available to the enzyme. For example, as free Hcy becomes scarce as a result of PEG htCBS activity, the balance between free Hcy and Hcy adducts (in the form of protein-bound Hcy or disulfides) in plasma, changes to favor the generation of free Hcy, which can further be processed by the enzyme.

A. Ocular Complications

Elevated Hcy levels are a strong and independent risk factor for ocular complications, in particular lens dislocation, in patients with HCU and in the general population. Even with prescribed pharmacologic and dietary interventions, the majority of HCU patients eventually present with ocular complications. Lowering Hcy levels has been shown to delay and perhaps prevent lens dislocation in HCU patients (see Yap S. Homocystinuria due to cystathionine beta-synthase deficiency. Orphanet Encyclopaedia [serial online] 2005; Mudd et al. Am J Hum Genet 1985; 37:1-31; Martinez-Gutierrez et al. Int Ophthalmol (2011) 31:227-232; Ajith et al. Clin Chim Acta 2015; 450:316-321; Mulvihill et al. J AAPOS 2001; 5:311-315; Marti-Carvajal et al. Cochrane Database Syst Rev 2015; 1:CD006612; Sweetser et al. N Engl J Med 2016, 375:1879-1890; Sadiq et al. Semin Ophthalmol 2013; 28:313-320; Wright et al. Homocysteine, folates, and the eye. Eye (Lond) 2008; 22:989-993; Lieberman et al. Am J Ophthalmol 1966, 61:252-255; Harrison et al. Ophthalmology 1998, 105:1886-1890; Ramsey et al. Am J Ophthalmol 1972; 74:377-385; Couser et al. Ophthalmic Genet 2017, 38:91-94; Ghorbanihaghjo et al. Mol Vis 2008, 14:1692-1697; Javadzadeh et al. Mol Vis 2010; 16:2578-2584; Seddon et al. Am J Ophthalmol 2006; 141:201-203; Coral et al. Eye (Lond) 2006; 20:203-207; Axer-Siegel et al. Am J Ophthalmol 2004, 137:84-89; Heuberger et al. Am J Clin Nutr 2002; 76:897-902; Huang et al. Sci Rep 2015; 5:10585; Sen et al. Indian J Clin Biochem 2008; 23:255-257; Yousefi et al. Protein Pept Lett 2013; 20:932-941; Gerth et al. J AAPOS 2008; 12:591-596; Stanger et al. Clin Chem Lab Med 2005, 43:1020-1025; Cahill et al. Am J Ophthalmol 2003, 136:1136-1150; Minniti et al. Eur J Ophthalmol 2014, 24:735-743; Turkcu et al. Medicina (Kaunas) 2013,49:214-218; Vessani et al. Am J Ophthalmol 2003; 136:41-46; Leibovitch et al. J Glaucoma 2003, 12:36-39; Leibovitzh et al. Medicine (Baltimore) 2016; 95:e4858; Micheal et al. Mol Vis 2009; 15:2268-2278; Clement et al. J Glaucoma 2009; 18:73-78; Cumurcu et al. BMC Ophthalmol 2006; 6:6; Bleich et al. J Neural Transm (Vienna) 2002; 109:1499-1504; Lee et al. Curr Eye Res 2017; 1-6; Wang et al. Am J Ophthalmol 2004; 137:401-406; Ganapathy et al. Invest Ophthalmol Vis Sci 2009; 50:4460-4470, each of which is hereby incorporated by reference in its entirety). Even with prescribed pharmacologic and dietary interventions, the majority of HCU patients eventually present with ocular complications. Lowering Hcy levels has been shown to delay and perhaps prevent lens dislocation in HCU patients (see Yap et al. J Inherit Metab Dis 1998; 21:738-747, which is hereby incorporated by reference in its entirety).

One of the most consistently present and earliest manifestations of HCU is ectopia lentis (lens dislocation) (see Mulvihill et al. J AAPOS 2001; 5:311-315, which is hereby incorporated by reference in its entirety). This usually occurs after the age of two years and is present in approximately 50% of untreated, pyridoxine non-responsive patients by the age of six years and in 50% of untreated pyridoxine-responsive patients by the age of 10 years (see Mudd et al. Am J Hum Genet 1985; 37:1-31, which is hereby incorporated by reference in its entirety). Dislocation may be partial (subluxation) or complete and, although it may occur inferiorly or nasally, it is usually bilateral (see Mulvihill et al. J AAPOS 2001; 5:311-315; Sweetser et al. N Engl J Med 2016; 375:1879-1890, both of which are hereby incorporated by reference in its entirety).

Lens dislocation often follows a period of rapidly progressing myopia, which can lead to marked astigmatism, monocular diplopia and decreased best-corrected acuity (see Sadiq et al. Semin Ophthalmol 2013; 28:313-320, which is hereby incorporated by reference in its entirety). Overall, myopia (greater than 1 diopter [D]) is believed to affect approximately 85% of HCU patients, with very high myopia (greater than 5D) affecting 50 to 76% of patients. Iridodonesis (quivering of the iris after moving the eyeball) affects approximately 56% of patients and spherophakia (a small, spherical lens that is prone to subluxation) affects 50% of patients (see Yap S. Homocystinuria due to cystathionine beta-synthase deficiency. Orphanet Encyclopaedia [serial online] 2005; Mulvihill et al. J AAPOS 2001; 5:311-315, each of which is hereby incorporated by reference in its entirety). Additional complications associated with HCU include cataract formation, chronic vitritis (inflammation of the vitreous humor) and chorioretinal inflammation, pupillary block with acute and/or chronic angle closure glaucoma and (in children), amblyopia (lazy eye) (see Sadiq et al. Semin Ophthalmol 2013; 28:313-320, which is hereby incorporated by reference in its entirety).

Evidence from a long-term, retrospective study in 25 patients under the age of 24 with HCU suggests that lens dislocation can be prevented, or at least significantly reduced and delayed, in patients whose tHcy levels are consistently lowered from an early age (see Yap et al. J Inherit Metab Dis 1998; 21:738-747, which is hereby incorporated by reference in its entirety). Early Hcy-lowering treatment was also associated with a reduced risk of overall ocular complications, including worsening myopia. Supportive evidence derives from a case-control study in 32 patients with HCU and 25 sibling controls, in which early Hcy-lowering treatment was associated with a significant reduction in ocular complications compared with patients who were treated later in life or were not fully compliant with treatment (see El Bashir et al. JIMD Rep 2015; 21:89-95, which is hereby incorporated by reference in its entirety).

The largest and longest longitudinal study to date of the ocular outcomes of 25 patients with cobalamin C deficiency, which is similarly characterized by elevated tHcy levels, found that macular degeneration, optic nerve pallor, nystagmus, strabismus, and vascular changes were all present in the majority of patients.

Numerous studies in HCU patients and in the general population demonstrated relationships between elevated Hcy levels and a variety of ocular disorders (see Mudd et al. Am J Hum Genet 1985; 37:1-31; Ajith, Clin Chim Acta 2015; 450:316-321; Mulvihill et al. J AAPOS 2001; 5:311-315; Wright et al. Eye (Lond) 2008; 22:989-993, each of which is hereby incorporated by reference in its entirety), including myopia and lens dislocation (see Yap S. Homocystinuria due to cystathionine beta-synthase deficiency. Orphanet Encyclopaedia [serial online] 2005; Mudd et al. Am J Hum Genet 1985; 37:1-31; Martinez-Gutierrez et al. Int Ophthalmol 2011; 31:227-232; Suri et al. J Neurol Sci 2014; 347:305-309; Mulvihill et al. J AAPOS 2001; 5:311-315; Lieberman et al. Am J Ophthalmol 1966; 61:252-255; Harrison et al. Ophthalmology 1998; 105:1886-1890; Ramsey et al. Am J Ophthalmol 1972; 74:377-385; Couser et al. Ophthalmic Genet 2017; 38:91-94; each of which is hereby incorporated by reference in its entirety), iridodonesis (see Mulvihill et al. J AAPOS 2001; 5:311-315, which is hereby incorporated by reference in its entirety), retinal arteriosclerosis (see Ghorbanihaghjo et al. Mol Vis 2008; 14:1692-1697, which is hereby incorporated by reference in its entirety), age-related macular degeneration (see Javadzadeh et al. Mol Vis 2010; 16:2578-2584; Seddon et al. Am J Ophthalmol 2006; 141:201-203; Coral et al. Eye (Lond) 2006; 20:203-207; Axer-Siegel et al. Am J Ophthalmol 2004; 137:84-89, each of which is hereby incorporated by reference in its entirety), age-related maculopathy (AMD) (see Heuberger et al. Am J Clin Nutr 2002; 76:897-902; Huang et al. Sci Rep 2015; 5:10585, both of which are hereby incorporated by reference in its entirety), cataracts (see Sen et al. Indian J Clin Biochem 2008; 23:255-257; Yousefi et al. Protein Pept Lett 2013; 20:932-941, both of which are hereby incorporated by reference in its entirety), diabetic retinopathy (possibly secondary to renal disease) (see Looker et al. Diabetologia 2003; 46:766-772; Pusparajah et al. Front Physiol 2016; 7:200, both of which are hereby incorporated by reference in its entirety) and macular and optic atrophy due to retinal vascular occlusion or non-arteritic ischemic optic neuropathy (see Gerth et al. J AAPOS 2008; 12:591-596; Stanger et al. Clin Chem Lab Med 2005; 43:1020-1025; Cahill et al. Am J Ophthalmol 2003; 136:1136-1150; Minniti et al. Eur J Ophthalmol 2014; 24:735-743; each of which is hereby incorporated by reference in its entirety).

A retrospective study of 629 patients with HCU found that lens dislocation usually occurs after the age of two years, and is present in approximately 50% of untreated, pyridoxine non-responsive patients by the age of six years and in 50% of untreated pyridoxine-responsive patients by the age of 10 years (see Mudd et al. Am J Hum Genet 1985; 37:1-31, which is hereby incorporated by reference in its entirety). The largest and longest longitudinal study to date of the ocular outcomes of 25 patients with cobalamin C deficiency, which is similarly characterized by elevated tHcy levels, found that macular degeneration, optic nerve pallor, nystagmus, strabismus, and vascular changes were all present in the majority of patients (see Brooks et al. Ophthalmology. 2016 March; 123(3):571-82, which is hereby incorporated by reference in its entirety).

Numerous studies in both HCU patients and the general population have demonstrated relationships between elevated Hcy levels and a variety of ocular disorders (see Mudd et al. Am J Hum Genet 1985; 37:1-31; Ajith T A, Ranimenon. Clin Chim Acta 2015; 450:316-321; Mulvihill et al. J AAPOS 2001; 5:311-315; Wright et al. Eye (Lond) 2008; 22:989-993; each of which is hereby incorporated by reference in its entirety), including myopia and lens dislocation (see Yap S. Homocystinuria due to cystathionine beta-synthase deficiency. Orphanet Encyclopaedia [serial online] 2005; Mudd et al. Am J Hum Genet 1985; 37:1-31; Martinez-Gutierrez et al. Int Ophthalmol 2011; 31:227-232; Mulvihill et al. J AAPOS 2001; 5:311-315; Sadiq et al. Semin Ophthalmol 2013; 28:313-320; Lieberman et al. Am J Ophthalmol 1966; 61:252-255; Harrison et al. Ophthalmology 1998; 105:1886-1890; Ramsey et al. Am J Ophthalmol 1972; 74:377-385; Couser et al. Ophthalmic Genet 2017; 38:91-94, each of which is hereby incorporated by reference in its entirety), iridodonesis (see Mulvihill et al. J AAPOS 2001; 5:311-315, which is hereby incorporated by reference in its entirety), retinal arteriosclerosis (see Ghorbanihaghjo et al. Mol Vis 2008; 14:1692-1697, which is hereby incorporated by reference in its entirety), age-related macular degeneration (see Javadzadeh et al. Mol Vis 2010; 16:2578-2584; Seddon et al. Am J Ophthalmol 2006; 141:201-203; Coral et al. Eye (Lond) 2006; 20:203-207; Axer-Siegel et al. Am J Ophthalmol 2004; 137:84-89; each of which is hereby incorporated by reference in its entirety), age-related maculopathy (AMD) (see Heuberger et al. Am J Clin Nutr 2002; 76:897-902; Huang et al. Sci Rep 2015; 5:10585, both of which are hereby incorporated by reference in its entirety), cataracts (see Sen et al. Indian J Clin Biochem 2008; 23:255-257; Yousefi et al. Protein Pept Lett 2013; 20:932-941, both of which are hereby incorporated by reference in its entirety), diabetic retinopathy (possibly secondary to renal disease) (see Looker et al. Diabetologia 2003; 46:766-772; Pusparajah et al. Front Physiol 2016; 7:200, both of which are hereby incorporated by reference in its entirety) and macular and optic atrophy due to retinal vascular occlusion or non-arteritic ischemic optic neuropathy (see Gerth et al. J AAPOS 2008; 12:591-596; Stanger et al. Clin Chem Lab Med 2005; 43:1020-1025; Cahill et al. Am J Ophthalmol 2003; 136:1136-1150; Minniti et al. Eur J Ophthalmol 2014; 24:735-743; each of which is hereby incorporated by reference in its entirety).

Studies investigating associations between Hcy levels and glaucoma provided inconsistent results. Some showed a positive relationship between Hcy levels and normal tension glaucoma, pseudoexfoliative glaucoma (PEXG) and primary open-angle glaucoma (POAG), while others did not (see Lieberman et al. Am J Ophthalmol 1966; 61:252-255; Turkcu et al. Medicina (Kaunas) 2013; 49:214-218; Vessani et al. Am J Ophthalmol 2003; 136:41-46; Leibovitch et al. J Glaucoma 2003; 12:36-39; Leibovitzh et al. Relationship between homocysteine and intraocular pressure in men and women: A population-based study. Medicine (Baltimore) 2016; 95:e4858; Micheal et al. Mol Vis 2009; 15:2268-2278; Clement et al. J Glaucoma 2009; 18:73-78; Cumurcu et al. BMC Ophthalmol 2006; 6:6; Bleich et al. J Neural Transm (Vienna) 2002; 109:1499-1504; Lee et al. Curr Eye Res 2017; 1-6; Wang et al. Am J Ophthalmol 2004; 137:401-406; each of which is hereby incorporated by reference in its entirety). However, loss of retinal ganglion cells (RGC)—a common observation in individuals with glaucoma—was demonstrated in mice with endogenously elevated Hcy levels caused by CBS gene deletion, suggesting a likely link between glaucoma and elevated tHcy levels in patients with HCU (see Ganapathy et al. Invest Ophthalmol Vis Sci 2009; 50:4460-4470, which is hereby incorporated by reference in its entirety).

1. Mechanism

A number of mechanisms have been proposed to explain effects of elevated Hcy levels on ocular health (see Ajith T A, Ranimenon; Clin Chim Acta 2015; 450:316-321, which is hereby incorporated by reference in its entirety). Mechanisms explaining the effects of elevated tHcy include impaired vascular endothelial function, apoptosis of retinal ganglion cells, extracellular matrix alterations, decreased lysyl oxidase activity and oxidative stress, as well as the direct cytotoxic and pro-inflammatory effects of Hcy, that appear to contribute to lens opacification and optic nerve damage.

Potential mechanisms also include activation of the N-methyl-D-aspartate (NMDA) receptor, leading to a cellular influx of calcium and increased reactive oxygen species (ROS) production, both of which contribute to cataract formation. These changes, along with the direct cytotoxic effects of Hcy, may cause endothelial injury, which initiates thrombogenesis and apoptosis of RGC, leading to retinopathy and glaucoma. Elevated Hcy levels have also been shown to increase levels of asymmetric dimethylarginine (AMDA) and block nitric oxide synthase (NOS) activity, thereby causing vasoconstriction and optic nerve atrophy by decreasing nitric oxide (NO) levels. Finally, an accumulation of homocysteinylated proteins on the vascular wall can trigger anti-Hcy antibody production and inflammatory responses, leading to phagocytosis, oxidative stress, apoptosis of RGCs and extracellular matrix (ECM) alterations. Together, such changes damage the vasculature, lens proteins and optic nerve, ultimately causing visual dysfunction.

In HCU patients, lens dislocation is regarded as being primarily caused by degenerative changes in zonular fibers, in particular Cys-rich, multidomain ECM proteins such as fibrillin-1 (see Sadiq et al. Semin Ophthalmol 2013; 28:313-320; Hubmacher et al. Biochemistry 2011; 50:5322-5332; Hubmacher et al. J Biol Chem 2005; 280:34946-34955; Hubmacher et al. J Biol Chem 2010; 285:1188-1198; each of which is hereby incorporated by reference in its entirety).

In healthy individuals, formation of numerous intra-domain disulfide bonds within fibrillin-1 enables precise protein folding, essential for structural integrity and function. Fibrillin-1 strands can then form inter-strand disulfide bonds, leading to assembly of high molecular weight multiprotein assemblies known as microfibrils (see Kinsey et al. J Cell Sci 2008; 121:2696-2704; Hubmacher et al. Proc Natl Acad Sci USA 2008; 105:6548-6553, both of which are hereby incorporated by reference in its entirety).

This process is highly dependent on interactions between fibrillin-1 and fibronectin (see Hubmacher et al. Biochemistry 2011; 50:5322-5332, which is hereby incorporated by reference in its entirety). Microfibrils form a scaffold for deposition of tropoelastin, an essential step in formation of elastic fibers such as those found in skin, lung, blood vessels/arteries, ligaments and the eye (see Hubmacher et al. J Biol Chem 2010; 285:1188-1198, which is hereby incorporated by reference in its entirety). The importance of fibrillin-1 is illustrated by patients with Marfan syndrome—a condition caused by mutation(s) in the fibrillin-1 gene—in which connective tissue dysfunction is associated with symptoms such as lens dislocation, organ prolapse, osteoporosis and joint hypermobility (see Suk et al. J Biol Chem 2004; 279:51258-51265; Collod-Beroud et al. Hum Mutat 2003; 22:199-208, both of which are hereby incorporated by reference in their entirety).

In vitro studies showed that addition of Hcy to fibrillin-1 disrupted disulfide bond formation, which in turn led to abnormal protein folding, increased susceptibility to proteolytic degradation and abnormal formation of ECM and elastic fibers (see Hubmacher et al. J Biol Chem 2010; 285:1188-1198; Whiteman et al. Antioxid Redox Signal 2006; 8:338-346, both of which are hereby incorporated by reference in their entirety). Addition of Hcy to human dermal fibroblasts was also associated with reduced forms of fibronectin that bound to fibrillin-1 sub-optimally, thereby preventing microfibril formation (see Hubmacher et al. Biochemistry 2011; 50:5322-5332; Hubmacher et al. J Biol Chem 2010; 285:1188-1198, both of which are hereby incorporated by reference in their entirety).

In addition to lens dislocation, degeneration of the zonular fibers in patients with HCU can lead to increased lens curvature, lenticular myopia, astigmatism, retinal detachment, strabismus, cataracts and iridodonesis (see Sadiq et al. Semin Ophthalmol 2013; 28:313-320, which is hereby incorporated by reference in its entirety). If untreated, anterior dislocation of the lens can cause acute pupillary block glaucoma. In extreme cases, complete lens dislocation is associated with increased ocular axial length, possibly a compensatory reaction to blurred vision (see Mulvihill et al. J AAPOS 2001; 5:311-315, which is hereby incorporated by reference in its entirety).

A retrospective study was performed on 25 HCU cases detected in Ireland between 1971 and 1996, either by the national NBS program or by clinical presentation, to examine the effects of Hcy-lowering therapies on clinical outcomes(see Yap et al. J Inherit Metab Dis 1998; 21:738-747, which is hereby incorporated by reference in its entirety). The majority of the cases (24/25) were pyridoxine non-responsive. Consequently, treatment of most patients consisted of a Met-free, Cys-supplemented diet, with vitamin B₁₂ and folate supplements, if required. Treatment was started before 6 weeks of age for patients and compared to a different group where treatment began upon diagnosis and one control patient who was never treated. The mean period of follow-up was 14.3 years (range 2.5 to 23.4) in groups treated before 6 weeks of age and 14.7 years (range 11.7 to 18.8) for the other patients, resulting in a total of 365.7 patient-years of treatment. Of the 21 patients detected by NBS, 18 remained free from complications during treatment. Of these individuals, 15/18 had 20:20 vision and 3/18 had had increasing myopia during the previous two years.

Consistent with the findings of Mudd et al. (see Mudd et al. Am J Hum Genet 1985; 37:1-31, which is hereby incorporated by reference in its entirety), lens dislocation in late-diagnosed individuals occurred at around two years of age. Lens dislocation was not reported in any of the early-treated individuals who had good compliance with therapy. Three of the ‘early-treated patients’ (those with the highest levels of fHcy) had worsening myopia without lens dislocation, which was most likely because of the relatively high fHcy levels in this small group of patients. This led the authors to suggest that progressive myopia might be the first sign, prior to lens dislocation, of poor dietary compliance, despite patient insistence to the contrary. The worsening myopia in these patients highlights how tenuous the balance is between neutral and negative clinical outcomes for these patients. Late detected patients all developed ectopia lentis. This suggests that treatment might delay the onset of lens dislocation, rather than prevent it.

Lifetime median plasma fHcy levels were higher in patients with myopia than in those without (18, 18 and 48 μmol/L vs 11 μmol/L, respectively). Of the three patients identified by NBS that developed complications in the group where treatment began upon diagnosis, all were non-compliant with their diets. Overall, 6/24 patients had lens dislocation; of these, two had an early diagnosis but were non-compliant with their diets and four had a late diagnosis, including the one patient that was never treated. Consistent with the findings of Mudd et al. (see Mudd et al. Am J Hum Genet 1985; 37:1-31, which is hereby incorporated by reference in its entirety), lens dislocation in late-diagnosed individuals (i.e. patients presenting with complications after age 2) occurred at approximately two years of age. At the time the study was published, lens dislocation had not been reported by any of the early-treated individuals with good compliance to therapy.

The compliant patients maintained their fHcy levels to tHcy equivalent levels largely below 120 μmol/L. However, all patients were under 24 years at the time of publication and many were still pediatric patients. Compliance with the protein restricted diet has been shown to rapidly decrease from adolescence through adulthood. The delicate balance described above suggests that the modest reduction of tHcy levels that was achieved by these patients may delay the onset of symptoms rather than prevent them as these patients age.

These results (see Yap et al. J Inherit Metab Dis 1998; 21:738-747; Mudd et al. Am J Hum Genet 1985; 37:1-31; each of which is hereby incorporated by reference in its entirety) were supported by those from a similar case-control study, conducted in Qatar, reporting on outcomes, including vision disturbances, in 32 cases of HCU and 25 sibling controls (see El Bashir et al. JIMD Rep 2015; 21:89-95, which is hereby incorporated by reference in its entirety). The mean age of the subjects was 11.2 years (range 0.6 to 29) and 56% were male. Overall, 9/32 cases (28%) were diagnosed by NBS and treated in the first month of life. The rest were diagnosed between 14 and 240 months of age. tHcy and Met levels were significantly lower among those diagnosed through NBS compared with those diagnosed clinically. This was possibly attributable to better compliance with diet and medications early in life. None of the 9 cases identified by NBS had vision problems at the time the study was published, compared with 18 (78%) in the late diagnosed group (p<0.001 between groups). However, similarly to the Irish study of 25 patients described above, patients in this study ranged from 0.6 to 29 years of age, and the long-term complications cannot be known yet.

A comparison of data from Yap and Naughten (see Yap et al. J Inherit Metab Dis 1998; 21:738-747, which is hereby incorporated by reference in its entirety) with the Kaplan-Meier curves produced by Mudd et al. showed that the proportion of treatment-compliant ‘early-treated’ patients with lens dislocation and osteoporosis was significantly lower than that expected for untreated patients with HCU (p≤0.001).

Therefore, an increased Hcy level is considered to be a strong and independent risk factor for ocular complications, in particular lens dislocation, in patients with HCU and in the general population (for example, as shown in Yap et al. and Mudd et al.). This highlights the need for early HCU diagnosis and treatment, as well as treatment compliance by the patient.

B. Skeletal Complications

HCU is associated with an increased risk of osteoporotic fractures that can be attributed partly to low bone mineral density (see Mudd et al. and Weber et al. Mol Genet Metab 2016; 117:351-354; each of which is hereby incorporated by reference in its entirety).

A retrospective chart review of data from 19 HCU patients over 8 years found that low bone mineral density (BMD) was common among both pediatric and adult HCU patients (see Weber et al.). This study suggested that accrual of bone mass during childhood and adolescence, a critical period for skeletal growth, is deficient in HCU and may negatively impact attainment of peak bone mass. This study also highlighted how even diet compliant patients with moderately elevated tHcy levels of only 5-fold above the normal range already suffer from poor skeletal clinical outcomes in childhood.

According to Mudd et al., 80% of patients with HCU develop osteoporosis before the age of 30 years. Moreover, elevated Hcy levels are associated with an increased risk of osteoporotic fractures, even in patients that do not have HCU (see; van Meurs et al. N Engl J Med 2004; 350:2033-2041; McLean et al. N Engl J Med 2004; 350:2042-2049; each of which is hereby incorporated by reference in its entirety).

A retrospective chart review of data from 19 subjects (9 males aged 3.5 to 49.2 years) undergoing clinical DXA bone densitometry between 2002 and 2010 found that low BMD was common among both pediatric and adult HCU patients (see Weber et al. Mol Genet Metab 2016; 117:351-354, which is hereby incorporated by reference in its entirety). At the time of the first DXA scan, the mean lumbar spine (LS) BMD Z-score was −1.2±1.3, and total hip BMD Z-score was −0.89±0.4; both were significantly lower than 0 (the expected mean Z-score in the general population) with p=0.002 and 0.02, respectively. The LS BMD Z-score at diagnosis was −1.26±1.4 in patients aged <21 years and −1.06±1.1 in adults. Overall, 38% of patients had low BMD for age (as defined by a Z-score ≤−2). Both tHcy and Met levels were positively associated with LS BMD Z-score in multiple linear regression models (see Weber et al. Mol Genet Metab 2016; 117:351-354, which is hereby incorporated by reference in its entirety). The mean tHcy levels for these 19 individuals was only 59.2 μmol/L, and the majority of the 19 patients were pediatric. This study suggests that accrual of bone mass during childhood and adolescence, a critical period for skeletal growth, is deficient in HCU and may negatively impact attainment of peak bone mass. This study also highlights how diet compliant patients with moderately elevated tHcy levels of only 5-fold above the normal range already suffer from poor skeletal clinical outcomes in childhood.

Previous studies have demonstrated clear relationships between Hcy levels and risk of fractures in elderly populations (see van Meurs et al. N Engl J Med 2004; 350:2033-2041; McLean et al. N Engl J Med 2004; 350:2042-2049; each of which is hereby incorporated by reference in its entirety). Results from two prospective, population-based studies, including 2406 subjects aged ≥55 years, showed that the age- and sex-adjusted risks of fracture were increased by 30% for each one SD increase in tHcy level (see van Meurs et al. N Engl J Med 2004; 350:2033-2041; which is hereby incorporated by reference in its entirety). A homocysteine level in the highest age-specific quartile was associated with an increase by a factor of 1.9 in the risk of fracture. The associations between homocysteine levels and the risk of fracture appeared to be independent of bone mineral density and other potential risk factors for fracture. An increased homocysteine level was a strong and independent risk factor for osteoporotic fractures in older men and women in the general population, similar in magnitude to that of established risk factors for fractures and for cardiovascular disease (see van Meurs et al. N Engl J Med 2004; 350:2033-2041; which is hereby incorporated by reference in its entirety). Furthermore, a US prospective study of 825 men and 1174 women (HOPE-2 trial sub-study) found that a serum tHcy level in the highest quartile was associated with a 1.9-fold increased risk of hip fractures among women and a four-fold increased risk among men, compared with serum tHcy levels in the lowest quartile (see Sawka et al. Arch Intern Med. 2007 Oct. 22; 167(19):2136-9, which is hereby incorporated by reference in its entirety). The associations between tHcy levels and fracture risk were independent of BMD and other potential risk factors for fracture (see van Meurs et al. N Engl J Med 2004; 350:2033-2041; McLean et al. N Engl J Med 2004,350:2042-2049; Sawka et al. Arch Intern Med. 2007 Oct. 22; 167(19):2136-9; each of which is hereby incorporated by reference in its entirety).

Consistent with these results, a study in 433 stroke patients, aged greater than 65 years, found that the age-adjusted incidence rates per 1000 person-years for hip fractures increased almost linearly from 2.89 in the lowest quartiles of Hcy levels to 27.87 in the highest quartiles (see). Together, these results suggest that increased Hcy levels are a strong and independent risk factor for osteoporotic fractures in older men and women.

Skeletal abnormalities are not present at birth and are unusual in infants and very young children (see Mudd et al. Am J Hum Genet 1985; 37:1-31, which is hereby incorporated by reference in its entirety). The first signs of skeletal involvement are usually genu valgum and pes cavus, with elongation of the long bones—a typical characteristic of Marfan syndrome—often developing close to puberty (see Morris et al. J Inherit Metab Dis 2017; 40:49-74; which is hereby incorporated by reference in its entirety). Osteoporosis, especially of the vertebrae and long bones, is common in HCU patients and may lead to scoliosis/kyphosis and/or vertebral collapse (see Mudd et al. Am J Hum Genet 1985; 37:1-31; Weber et al. Mol Genet Metab 2016; 117:351-354; each of which is hereby incorporated by reference in its entirety). Other skeletal manifestations may include Marfanoid facial features caused by prominent upper teeth and a high palate and anterior chest wall deformities, such as pectus excavatum or carinatum (see Morris et al. J Inherit Metab Dis 2017; 40:49-74; Sweetser et al. N Engl J Med 2016; 375:1879-1890; Brenton et al. J Bone Joint Surg Br 1972; 54:277-298; each of which is hereby incorporated by reference in its entirety). Because of these shared skeletal characteristics between Marfan syndrome and HCU, HCU patients are sometimes mischaracterized as Marfan patients.

A study in 25 Irish patients with HCU followed over 25 years found that the risk of osteoporosis was considerably lower in patients identified through newborn screening with good Hcy-lowering treatment compliance (diet, vitamins, and/or betaine), compared with non-compliant patients or in those with a late diagnosis (see Yap et al. J Inherit Metab Dis 1998; 21:738-747). Supportive evidence for these results came from a small Korean study in five HCU patients with good, long-term metabolic control. In this study, patients receiving early Hey-lowering therapy had fewer skeletal abnormalities than those with a later diagnosis (see Lim et al. Osteoporos Int 2013,24:2535-2538, which is hereby incorporated by reference in its entirety). Finally, in a study using a murine model for HCU, normalization of tHcy levels by treatment with a CBS ET was associated with osteoporosis prevention (see Majtan et al. Enzyme replacement prevents neonatal death, liver damage, and osteoporosis in murine homocystinuria, FASEB J 2017, which is hereby incorporated by reference in its entirety).

The precise mechanisms leading to low BMD and skeletal fragility in patients with HCU are not fully understood (see Weber et al. Mol Genet Metab 2016; 117:351-354; Lim J S, Lee D H. Changes in bone mineral density and body composition of children with well-controlled homocystinuria caused by CBS deficiency. Osteoporos Int 2013; 24:2535-2538, both of which are hereby incorporated by reference in its entirety). However, many of the connective tissue disorders in patients with HCU resemble those seen in Marfan Syndrome, a connective tissue disorder caused by mutations in the fibrillin-1 gene and characterized by features including elongation of the long bones and osteoporosis-type fractures (see Hubmacher et al. Biochemistry 2011; 50:5322-5332; Hubmacher et al. J Biol Chem 2010; 285:1188-1198, both of which are hereby incorporated by reference in its entirety). Elevated Hcy levels are believed to lead to bone fragility and fractures via two distinct pathways (see Behera et al. J Cell Physiol 2016, which is hereby incorporated by reference in its entirety). The first results in reduced accrual of bone mass during childhood and adolescence via impaired fibrillin assemblies. The second pathway leads to impaired bone remodeling, resulting in brittle bones via decreased collagen crosslink formation (see Behera et al. J Cell Physiol 2016; Kang et al. J Clin Invest 1973; 52:2571-2578, both of which are hereby incorporated by reference in its entirety). Together, these data suggest that accrual of bone mass during childhood and adolescence, a critical period for skeletal growth, is deficient in patients with HCU and that this negatively affects attainment of peak bone mass.

Moreover, a strong relationship exists between Hcy levels and risk of fracture, in elderly populations (see van Meurs et al. N Engl J Med 2004; 350:2033-2041; McLean et al. N Engl J Med 2004; 350:2042-2049; each of which is hereby incorporated by reference in its entirety). Results from two international, prospective, population-based studies, including 2,406 subjects aged greater than or equal to 55 years, showed that a homocysteine level in the highest age-specific quartile was associated with an increase by a factor of 1.9 in the risk of fracture (see van Meurs et al. N Engl J Med 2004; 350:2033-2041, which is hereby incorporated by reference in its entirety). An increased homocysteine level was a strong and independent risk factor for osteoporotic fractures in older men and women in the general population, similar in magnitude to that of established risk factors for fractures (low bone mineral density, cognitive impairment, recent falls) and for cardiovascular disease (see van Meurs et al. N Engl J Med 2004; 350:2033-2041, which is hereby incorporated by reference in its entirety). Furthermore, a US prospective study of 1,999 subjects (HOPE-2 trial sub-study) found that a serum tHcy level in the highest quartile was associated with a 1.9-fold increased risk of hip fractures among women and a four-fold increased risk among men, compared with serum tHcy levels in the lowest quartile (see Sawka et al. Arch Intern Med. 2007 Oct. 22; 167(19):2136-9, which is hereby incorporated by reference in its entirety). The associations between tHcy levels and fracture risk were independent of BMD and other potential risk factors for fracture (see van Meurs et al. N Engl J Med 2004; 350:2033-2041; McLean et al. N Engl J Med 2004; 350:2042-2049; Sawka et al. Arch Intern Med. 2007 Oct. 22; 167(19):2136-9, each of which is hereby incorporated by reference in its entirety). Consistent with these results, a study in 433 stroke patients, aged >65 years, found that the age-adjusted incidence rates per 1000 person-years for hip fractures increased almost linearly from 2.89 in the lowest quartiles of Hcy levels to 27.87 in the highest quartiles 0. Together, these results suggest that increased Hcy levels are a strong and independent risk factor for osteoporotic fractures in older men and women.

The precise mechanisms leading to low BMD and skeletal fragility in patients with HCU are not fully understood (see Weber et al. Mol Genet Metab 2016; 117:351-354; Lim JS, Lee DH. Changes in bone mineral density and body composition of children with well-controlled homocystinuria caused by CBS deficiency. Osteoporos Int 2013; 24:2535-2538, both of which are hereby incorporated by reference in its entirety). However, many of the connective tissue disorders in patients with HCU resemble those seen in Marfan Syndrome, a connective tissue disorder caused by mutations in the fibrillin-1 gene and characterized by features including elongation of the long bones and osteoporosis-type fractures (see Brenton et al. J Bone Joint Surg Br 1972; 54:277-298; Hubmacher et al. Biochemistry 2011; 50:5322-5332; Hubmacher et al. J Biol Chem 2010; 285:1188-1198, each of which is hereby incorporated by reference in its entirety).

In healthy individuals, fibrillin-1, together with collagen and elastin polymers, assembles to form the ECM, the architectural scaffolds for bone formation, homeostasis and repair (see Olivieri et al. Fibrogenesis Tissue Repair 2010; 3:24, which is hereby incorporated by reference in its entirety). Studies show that elevated tHcy levels can lead to structural modifications of fibrillin-1 fragments, which prevent multimerization and lead to fibrillin-1 degradation (see Hubmacher et al. J Biol Chem 2005; 280:34946-34955; Hubmacher et al. J Biol Chem 2010; 285:1188-1198, both of which are hereby incorporated by reference in its entirety). This process is further impaired by the homocysteinylation of fibronectin, which prevents the formation of fibronectin-fibrillin complexes necessary for fibrillin-1 multimerization (see Hubmacher et al. Biochemistry 2011; 50:5322-5332, which is hereby incorporated by reference in its entirety). Such findings suggest that elevated Hcy levels have a detrimental effect on ECM formation.

In healthy individuals, fibrillin assemblies (i.e. microfibrils) play an important role in bone mineralization, through storage and activation of transforming growth factor-beta (TGF-beta) and bone morphogenetic proteins (BMP) (see Nistala et al. Ann N Y Acad Sci 2010; 1192:253-256; Nistala et al. J Biol Chem 2010; 285:34126-34133, both of which are hereby incorporated by reference in its entirety). Impaired activation of TGF-beta and BMPs could potentially contribute to the skeletal phenotype observed in both Marfan syndrome and HCU and might also decrease bone mineral content, as observed in mild forms of HCU (see Herrmann et al. Clin Chem 2005; 51:2348-2353, which is hereby incorporated by reference in its entirety). Moreover, there is in vivo and in vitro evidence that Hcy may weaken bone strength through decreased collagen crosslink formation (see Kang et al. J Clin Invest 1973; 52:2571-2578, which is hereby incorporated by reference in its entirety). Together, these data suggest that accrual of bone mass during childhood and adolescence, a critical period for skeletal growth, is deficient in patients with HCU and that this may negatively impact attainment of peak bone mass.

In addition to its effects on bone deposition, elevated Hcy levels increase the rate of bone remodeling by increasing osteoclast (OC) activity and decreasing osteoblast (OB) activity (see Behera et al. J Cell Physiol 2016; Herrmann et al. Clin Chem 2005; 51:2348-2353; Vacek et al. Clin Chem Lab Med 2013; 51:579-590; Vijayan et al. J Endocrinol 2017; 233:243-255, each of which is hereby incorporated by reference in its entirety). An imbalance between OB and OC activities can lead to brittle bones and an increased incidence of fractures. Mechanisms leading to Hcy-mediated decreases in OB activity are believed to include decreased bone blood flow (a consequence of decreased NO availability) (see Tyagi et al. Vasc Health Risk Manag 2011; 7:31-35, which is hereby incorporated by reference in its entirety) and increased rates of OB apoptosis (see Behera et al. J Cell Physiol 2016; Kim et al. Bone 2006; 39:582-590, both of which are hereby incorporated by reference in their entireties). Mechanisms leading to enhanced OC activity are believed to include increased levels of intracellular ROS, which enhance both OC differentiation and OC activity via increased matrix metalloproteinase (MMP) activity (see Vacek et al. Clin Chem Lab Med 2013; 51:579-590, which is hereby incorporated by reference in its entirety) and suppression of OC apoptosis (see Behera et al. J Cell Physiol 2016; Herrmann et al. Clin Chem 2005; 51:2348-2353; Koh et al. J Bone Miner Res 2006; 21:1003-1011, each of which is hereby incorporated by reference in its entirety). Indeed, a recent study in CD1 mice fed a high Hcy diet showed that short-term (7 day) Hcy administration was associated with a loss of tissue mineral density (TMD) and increased OC numbers, whereas long-term Hcy administration (30 days) led to OC reprogramming, apoptosis and mineralization, which reinstated TMD but compromised tissue biomechanical properties (see Vijayan et al. J Endocrinol 2017; 233:243-255, which is hereby incorporated by reference in its entirety).

Thus, elevated Hcy levels can lead to bone fragility and fractures via two distinct pathways (see Behera et al. J Cell Physiol 2016, which is hereby incorporated by reference in its entirety). The first results in reduced accrual of bone mass during childhood and adolescence, via impaired ECM formation and suppressed activation of fibrillin-1-associated TGF-beta and BMP. The second pathway leads to impaired bone remodeling, resulting in brittle bones, via increased OC and decreased OB activities.

Elevated Hcy levels are associated with increased oxidative stress in the bone microenvironment. Increased ROS induces osteoblast apoptosis, thereby decreasing osteoblast genesis. This increase in oxidative stress further decreases NO availability through production of superoxide anions, which might also decrease bone blood flow and angiogenesis. The ROS generated by this process activates osteoclast genesis by monocyte fusion, further contributing to loss of BMD, leading to osteoporosis.

A recent study in newborn CBS knockout (KO) mice, maintained on standard rodent chow without Met restriction, found that subcutaneous administration of a CBS ET, using recombinant PEGylated human truncated CBS (PEG-CBS) for 5 months, prevented the reduction in bone mineral density in these animals, and could also normalize these values in animals that were treated later in life (see Majtan et al. Enzyme replacement prevents neonatal death, liver damage, and osteoporosis in murine homocystinuria. FASEB J 2017, which is hereby incorporated by reference in its entirety). In this study, changes in body composition that characterize both the KO model and HCU patients were prevented. In both plasma and tissues, tHcy and Cys levels were normalized, Cth levels increased and SAM/SAH ratios improved.

Supportive evidence for the effects of Hcy-lowering on skeletal outcomes derives from a 25-year survey of 25 Irish patients with HCU (see Yap et al. J Inherit Metab Dis 1998; 21:738-747, which is hereby incorporated by reference in its entirety). In this study, osteoporosis (diagnosed by radiological examination, rather than DXA) was present in one of the three treatment non-compliant patients identified by NBS and in one of the four patients with a late diagnosis (at two years of age). None of the 18 patients who had been compliant with early treatment (from 6 weeks of age) showed signs of osteoporosis.

A small study was conducted in Korea in five HCU patients (3 boys and 3 girls), all diagnosed at young age (3 during NBS and 2 at age 7 years), with good metabolic control for 3.4 years (see Lim et al. Osteoporos Int 2013; 24:2535-2538, which is hereby incorporated by reference in its entirety). Mean plasma tHcy level at diagnosis was 34.3±52.6 (13 to 78.6) μmol/L. plasma Met was 716±1347.6 (24.3 to 1566) μmol/L and treatment comprised a low-Met diet with pyridoxine, betaine and folic acid supplementation. Body composition measurements and BMDs for all patients were within normal ranges for the Korean population, and no significant changes were observed in skeletal morphology over time. Three patients (60%) had mild scoliosis of the T-L spine (Cobb angles 7.3°, 7.6° and 10.3°), and fractures were reported four times in three patients. Of these, two were caused by a sports injury and one by a traffic accident. Two cases of mild compression fracture of the lumbar spine were detected by radiography and a history of severe back pain was documented. Patients receiving an early diagnosis showed fewer skeletal abnormalities than those with a later diagnosis. This study showed however, that even patients receiving an early diagnosis by NBS, who were compliant with dietary treatment and had only slight to moderately elevated levels of tHcy, already displayed skeletal abnormalities and multiple fractures as children.

Together, these findings suggest a beneficial effect of early Hcy-lowering treatment on skeletal outcomes in patients with HCU. It should be noted that in treatment-compliant patients tHcy levels were lowered but not normalized, and though fewer skeletal abnormalities were present in these patients, significant negative clinical outcomes (osteoporosis and fractures) were noted in this largely young patient group.

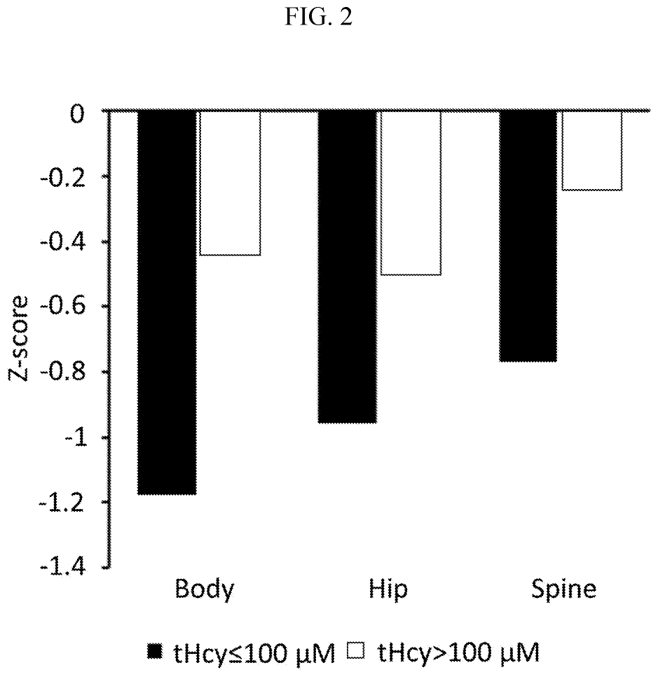

Present studies demonstrate positive correlations between BMD and Z-scores and plasma tHcy and dietary protein intake in HCU patients, suggesting that insufficient protein intake may play a role in increasing skeletal fragility in these patients. Patients with plasma tHcy levels <100 μM had much greater skeletal fragility than those with levels >100 μM (Example 6; FIG. 2). There was a positive correlation between plasma tHcy levels and BMD at all three bone locations tested (Pearson's r=0.33 to 0.51; p<0.03; Table 8). Moreover, higher BMD is correlated with greater total dietary protein intake. Indeed, chronically restricting total protein intake to control tHcy levels may increase patient skeletal fragility.

BMD and Z-scores assessed by dual-energy X-ray absorptiometry (DXA) as described herein (see Example 6) can be a reliable endpoint to assess the efficacy of treatments in HCU patients. Thus, the methods of treatment comprising enzyme therapy as described herein can be used to relax HCU patient restricted diets, permitting increased protein consumption and improving skeletal fragility outcomes in patients. ET, as described herein, in combination with BMD and Z-score assessment, can be used to ascertain patient tolerability to relaxed diet and/or need for restricted diet.

C. Vascular Complications

The relationship between HCU and vascular disease was first demonstrated in 1985 in an epidemiological study in patients with moderate to severely elevated Hcy levels due to homozygous HCU (see Mudd et al. Am J Hum Genet 1985; 37:1-31, which is hereby incorporated by reference in its entirety).

Thromboembolism is the major cause of morbidity and premature death in HCU patients (see Mudd et al. Am J Hum Genet 1985; 37:1-31; Karaca et al. Gene 2014; 534:197-203; Yap S. J Inherit Metab Dis 2003; 26:259-265, each of which is hereby incorporated by reference in its entirety). The overall rate of thromboembolic events in patients with untreated HCU is approximately 10% per year (see Cattaneo M. Semin Thromb Hemost 2006; 32:716-723, which is hereby incorporated by reference in its entirety), with risk increasing after surgery and during or immediately after pregnancy (see Mudd et al. Am J Hum Genet 1985; 37:1-31; Novy et al. Thromb Haemost 2010; 103:871-873, both of which are hereby incorporated by reference in its entirety). Thromboembolism can affect any blood vessel, but venous thrombosis (in particular CSVT) is more common than arterial thrombosis in patients with HCU (see Mudd et al. Am J Hum Genet 1985; 37:1-31; Karaca et al. Gene 2014; 534:197-203; Eslamiyeh et al. Iran J Child Neurol 2015; 9:53-57; Saboul et al. J Child Neurol 2015; 30:107-112, each of which is hereby incorporated by reference in its entirety). Cerebrovascular accidents, especially CSVT, have been described in infants (see Mahale et al. J Pediatr Neurosci. 2017 April-June; 12(2):206-207, which is hereby incorporated by reference in its entirety), although more typically appear in young adults (see Yap et al. Arterioscler Thromb Vasc Biol 2001; 21:2080-2085, which is hereby incorporated by reference in its entirety).

The risk of thromboembolic events was approximately 25% by age 16 years and 50% by age 29 years. In 1999, Hankey et al. reported that all three genetic causes of HCU (HCU, MTHFR deficiency and vitamin B12 deficiency) were associated with a high risk of premature cardiovascular (CV) disease, affecting half of all homozygotes by the age of 30 years (see Hankey et al. Lancet 1999; 354:407-413, which is hereby incorporated by reference in its entirety). The only biochemical change common to all three disorders are elevated serum Hcy levels (often greater than 100 μmol/L) (see Faeh et al. Swiss Med Wkly 2006; 136:745-756, which is hereby incorporated by reference in its entirety). Several reports described how treatments decreasing tHcy levels significantly reduced the incidence of vascular events, the main cause of morbidity, in HCU patients (see Yap et al. J Inherit Metab Dis 2001; 24:437-447; Wilcken DE, Wilcken B. The natural history of vascular disease in homocystinuria and the effects of treatment. J Inherit Metab Dis 1997; 20:295-300, both of which are hereby incorporated by reference in its entirety). Since then, a number of other studies demonstrated an increased risk of vascular events, in particular venous thrombosis, in HCU patients (see Karaca et al. Gene 2014; 534:197-203; Kelly et al. Neurology 2003; 60:275-279; Lussana et al. Thromb Res 2013; 132:681-684; Magner et al. J Inherit Metab Dis 2011; 34:33-37, each of which is hereby incorporated by reference in its entirety).

An elevated plasma tHcy level is a risk factor for vascular disease and a strong predictor of mortality in patients with coronary artery disease, both with and without HCU (see Mudd et al. Am J Hum Genet 1985; 37:1-31; Karaca et al. Gene 2014; 534:197-203; Kelly et al. Neurology 2003; 60:275-279; Faeh et al. Swiss Med Wkly 2006; 136:745-756; Boushey et al. JAMA 1995; 274:1049-1057; Clarke R et al. JAMA 2002; 288:2015-2022; Hankey et al. Lancet 1999; 354:407-413; Khan et al. Stroke 2008; 39:2943-2949; Graham et al. The European Concerted Action Project. JAMA 1997; 277:1775-1781; Clarke et al. N Engl J Med 1991; 324:1149-1155; Clarke et al. Ir J Med Sci 1992; 161:61-65; Woodward et al. Blood Coagul Fibrinolysis 2006; 17:1-5; Refsum et al. Annu Rev Med 1998; 49:31-62; Yoo et al. Stroke 1998; 29:2478-2483; Selhub et al. N Engl J Med 1995; 332:286-291; Wald et al. BMJ 2002; 325:1202; Bautista et al. J Clin Epidemiol 2002; 55:882-887; Brattstrom et al. Atherosclerosis 1990; 81:51-60; Lussana et al. Thromb Res 2013; 132:681-684; Casas et al. Lancet 2005; 365:224-232; McCully KS. Am J Pathol 1969; 56:111-128; Magner et al. J Inherit Metab Dis 2011; 34:33-37; Wilcken et al. J Clin Invest 1976; 57:1079-1082; Nygard et al. N Engl J Med 1997; 337:230-236; each of which is hereby incorporated by reference in its entirety). Although there is evidence for a relationship between tHcy levels and CV risk (see Boushey et al. JAMA 1995; 274:1049-1057, which is hereby incorporated by reference in its entirety), the relationships between tHcy and stroke/peripheral arterial disease are considerably stronger (see Clarke et al. JAMA 2002; 288:2015-2022; Khan et al. Stroke 2008; 39:2943-2949; Wald et al. BMJ 2002; 325:1202; Casas et al. Lancet 2005; 365:224-232; Brattstrom et al. Haemostasis 1989; 19 Suppl 1:35-44; each of which is hereby incorporated by reference in its entirety). Although large studies (NORVIT, HOPE-2, VITATOPS) in the general population initially concluded that lowering Hcy levels had a minor effect on major vascular events and recurrent cardiovascular disease, a further, more specific analysis of the data has clearly shown the clinical benefits of tHcy reduction on stroke.

There is considerable evidence that Hcy-lowering decreases stroke risk in the general population with mildly elevated tHcy levels (see Saposnik et al. Stroke 2009; 40:1365-1372; Huo et al. JAMA 2015; 313:1325-1335; Lonn et al. N Engl J Med 2006; 354:1567-1577; Hankey et al. Lancet Neurol 2012; 11:512-520; Spence J D, Lancet Neurol. 2007 September; 6(9):830-8, each of which is hereby incorporated by reference in its entirety). In the HOPE-2 study (see Saposnik et al. Stroke 2009; 40:1365-1372, which is hereby incorporated by reference in its entirety) of 5,552 patients, minor reductions in tHcy levels (3 mmol/L vs placebo) led to a significant reduction in stroke incidence (27% relative risk reduction, 1.3% absolute risk reduction), suggesting that even small decreases in tHcy levels can be beneficial. This effect was most pronounced in patients with baseline Hcy in the upper quartile who had a 4.3% absolute risk reduction. Although it is unclear whether Hcy-lowering affects overall CV outcomes in patients with mildly elevated tHcy and without HCU (see Marti-Carvajal et al. Cochrane Database Syst Rev 2015; 1:CD006612, which is hereby incorporated by reference in its entirety), its vascular benefits have been consistently demonstrated in patients with HCU (see Yap et al. J Inherit Metab Dis 1998; 21:738-747; Eslamiyeh et al. Iran J Child Neurol 2015; 9:53-57; Saboul et al. J Child Neurol 2015; 30:107-112; Yap et al. Arterioscler Thromb Vasc Biol 2001; 21:2080-2085; Woods et al. BMJ Case Rep 2017; Wilcken et al. J Inherit Metab Dis 1997; 20:295-300; Yap et al. Semin Thromb Hemost 2000; 26:335-340; Ruhoy et al. Pediatr Neurol 2014; 50:108-111, each of which is hereby incorporated by reference in its entirety).

1. Mechanism

A number of studies have shown that elevated Hcy levels contribute to development of atherosclerosis or thrombosis through oxidative stress associated mechanisms (see Faverzani et al. Cell Mol Neurobiol 2017; Nowak et al. Arterioscler Thromb Vasc Biol 2017; 37:e41-e52; Vanzin et al. Mol Genet Metab 2011; 104:112-117; Vanzin et al. Gene 2014; 539:270-274; Vanzin et al. Cell Mol Neurobiol 2015; 35:899-911; each of which is hereby incorporated by reference in its entirety), including inflammatory and immune activation via NF-ηB (see Rodriguez-Ayala et al. Atherosclerosis 2005; 180:333-340; van Guldener et al. Curr Hypertens Rep 2003; 5:26-31, both of which are hereby incorporated by reference in its entirety). Medial damage leading to thrombosis is believed to be caused by Hcy-mediated endothelial dysfunction (see Jiang et al. Arterioscler Thromb Vasc Biol 2005; 25:2515-2521; Hossain et al. J Biol Chem 2003; 278:30317-30327; Cai et al. Blood 2000; 96:2140-2148; Zhang et al. J Biol Chem 2001; 276:35867-35874; Papapetropoulos et al. Proc Natl Acad Sci U S A 2009; 106:21972-21977; Szabo et al. Br J Pharmacol 2011; 164:853-865; Chiku et al. J Biol Chem 2009; 284:11601-11612; Wang et al. Antioxid Redox Signal 2010; 12:1065-1077; Saha et al. FASEB J 2016; 30:441-456; Ebbing et al. JAMA 2008; 300:795-804; Bonaa et al. N Engl J Med 2006; 354:1578-1588; Marti-Carvajal et al. Cochrane Database Syst Rev 2015; 1:CD006612; Celermajer et al. J Am Coll Cardiol 1993; 22:854-858; Rubba et al. Metabolism 1990; 39:1191-1195; each of which is hereby incorporated by reference in its entirety), enhanced coagulation pathways (see Spence J D. Int J Stroke. 2016 October; 11(7):744-7; Fryer et al. Arterioscler Thromb 1993; 13:1327-1333; Lentz et al. J Clin Invest 1991; 88:1906-1914; each of which is hereby incorporated by reference in its entirety) and increased vascular dilatation. Such pro-thrombotic mechanisms are similar to those observed in Marfan patients (see Kelly et al. Neurology 2003; 60:275-279; Tripathi P. International Cardiovascular Forum J 2016; 6:13; van Guldener et al. Curr Hypertens Rep 2003; 5:26-31; Hackam et al. JAMA 2003; 290:932-940; Baumbach et al. Circ Res 2002; 91:931-937; Evangelisti et al. Int J Cardiol 2009; 134:251-254; de Valk et al. Stroke 1996; 27:1134-1136, each of which is hereby incorporated by reference in its entirety).

A causal relationship between tHcy levels and CV risk derived from a meta-analysis of data from 27 studies (more than 4,000 patients) showed a graded risk for atherosclerosis of CV, cerebrovascular and peripheral vessels, such that a 5 μM increase in Hcy conferred a 80% increased risk to women and a 60% increased risk to men (see Boushey et al. JAMA 1995; 274:1049-1057, which is hereby incorporated by reference in its entirety). A meta-analysis of data from 30 prospective or retrospective studies involving 5073 ischemic heart disease (IHD) events and 1113 strokes found that a 25% lower than usual (corrected for regression dilution bias) Hcy level (approximately 3 μmol/L) was associated with an 11% (odds ratio (OR), 0.89; 95% CI, 0.83 to 0.96) lower IHD risk and a 19% (OR, 0.81; 95% CI, 0.69 to 0.95) lower stroke risk (see Clarke R, et al. JAMA 2002; 288:2015-2022, which is hereby incorporated by reference in its entirety).

A study in patients with and without pre-existing vascular disease demonstrated an increase, after Met loading, of plasma Hcy (exceeding the highest values in comparable healthy control subjects) in 1/21 subjects with MI (5%), 14/37 subjects with aorto-iliac disease (38%) and 17/53 subjects with cerebrovascular disease (32%). This suggests that the links between Hcy levels and peripheral arterial disease (PAD) and stroke are considerably greater than the link between Hcy levels and MI (see Brattstrom et al. Haemostasis 1989; 19 Suppl 1:35-44, which is hereby incorporated by reference in its entirety). An independent, graded association between Hcy levels and stroke was described in a prospective study conducted among a UK cohort of 457 stroke patients and 179 control subjects from the same community (see Khan et al. Stroke 2008; 39:2943-2949). The highest Hcy levels were seen in patients with small vessel disease (SVD) (16.2 versus 11.8 μmol/L in control subjects without stroke, p<0.001 after adjusting for age, gender, vascular risk factors, vitamin levels and renal function). Within SVD cases, the highest Hcy levels were observed in individuals with lacunar infarction with confluent leukoaraiosis. Moreover, there was a correlation between Hcy levels and leukoaraiosis severity (r=−0.225; p<0.001).

These findings were further supported by a Mendelian randomization study, demonstrating a genetic association between MTHFR polymorphisms regulating Hcy metabolism and stroke risk (see Casas et al. Lancet 2005; 365:224-232, which is hereby incorporated by reference in its entirety). A literature search for all relevant studies on associations between Hcy levels and the MTHFR TT and CC polymorphisms on stroke risk identified 111 studies, including 15,635 individuals without cardiovascular disease (CVD). The weighted mean difference in Hcy levels between TT and CC homozygotes was 1.93 μmol/L (95% CI 1.38 to 2.47). Based on results from a previous meta-analysis of prospective studies, in which a 5 μmol/L increase in plasma Hcy levels corresponded to an OR for stroke of 1.59 (1.29 to 1.96), a 1.93 μmol/L increase in Hcy levels in healthy individuals with the TT genotype would result in an expected OR for stroke of 1.20 (1.10 to 1.31) (see Wald et al. BMJ 2002; 325:1202, which is hereby incorporated by reference in its entirety). Consistent with this result, Khan et al. reported a 1.26 (1.14 to 1.40) OR for stroke for TT versus CC homozygotes (p=0.29), irrespective of age group, ethnicity or geographical location. Together, these results suggested a causative role for elevated Hcy levels in stroke pathogenesis in the general population.

Whether moderate increases in serum Hcy typical in patients with heterozygous HCU because vascular disease has been examined in studies much smaller than those conducted in the general population. According to Mudd et al., the risk of vascular events in patients with mildly elevated tHcy levels due to heterozygous HCU (<5% by age 50 years) is similar to that in the general population (see Mudd et al. Am J Hum Genet 1981; 33:883-893, which is hereby incorporated by reference in its entirety). Consistent with this, an ultrasound study in individuals with homozygous and heterozygous HCU found impaired endothelial function in the systemic arteries of homozygous children as young as four years of age, whereas endothelial function was largely unaffected in heterozygous adults (see Celermajer et al. J Am Coll Cardiol 1993; 22:854-858, which is hereby incorporated by reference in its entirety).

Although a similar study demonstrated signs of premature arterial disease in both homozygotes and heterozygotes, individuals with the homozygous disorder developed signs at a much younger age (19 years versus 45 years) and disease severity was considerably greater (see Rubba et al. Metabolism 1990; 39:1191-1195, which is hereby incorporated by reference in its entirety).

Overall, observations in HCU patients were consistent with those from previous studies, with vascular risk increasing with Hcy levels (see Boushey et al. JAMA 1995; 274:1049-1057; Clarke et al. JAMA 2002; 288:2015-2022; Khan et al. Stroke 2008; 39:2943-2949; each of which is hereby incorporated by reference in its entirety).

These results suggest that an elevated Hcy level is a risk factor for CV disease. In addition, lowering Hcy levels has been shown to significantly reduce the risk of stroke in the general population and in patients with HCU.

Studies have shown that elevated Hcy levels caused by HCU can potentially contribute to development of atherosclerosis and/or thrombosis via various mechanisms. These include molecular events such as induction of oxidative stress and its downstream effects such as activation of NF-ηB (nuclear factor kappa-light-chain-enhancer of activated B cells), a transcriptional factor regulating proinflammatory and other damage-associated genes. Various Hcy-mediated effects modulating the physio-chemical properties of the vascular wall, such as those leading to endothelial dysfunction or arterial stiffness, could contribute to development of hypertension, thrombosis or other vascular abnormalities. Finally, there is evidence for direct induction of coagulation pathways by Hcy, a more direct route leading to thrombosis (see Faverzani et al. Cell Mol Neurobiol 2017; Hainsworth et al. Biochim Biophys Acta 2016; 1862:1008-1017; Ganguly et al. Nutr J 2015; 14:6; Tripathi P. Molecular and biochemical aspects of homocysteine in cardiovascular diseases. International Cardiovascular Forum J 2016; 6:13; Fryer et al. Arterioscler Thromb 1993; 13:1327-1333; each of which is hereby incorporated by reference in its entirety). Following a brief description of the molecular and biochemical mechanisms of atherosclerosis, the subsections below will review potential mechanisms leading to vascular disease in individuals with elevated tHcy levels, including those with HCU.

2. Atherosclerosis

Among the most well studied conditions leading to thrombosis and, consequently, vascular blockage, is atherosclerosis, a progressive inflammatory disease affecting coronary, cerebral and peripheral circulations (see Libby et al. Circulation 2005; 111:3481-3488, which is hereby incorporated by reference in its entirety). In its early stages, vascular injury leads to endothelial cell (EC) activation, monocyte recruitment into the intima and macrophage activation. An inflammatory atherosclerotic lesion (the fatty streak), comprising monocyte-derived, lipid-laden macrophages (foam cells) and T-lymphocytes, is formed. Progressive lipid accumulation forms a lipid core surrounded by a fibrous cap. During the later stages, activated macrophages secrete enzymes that weaken the fibrous cap, leading to plaque rupture, hemorrhage or thrombosis and ischemic attacks/acute coronary syndrome. Plaque rupture exposes tissue factor to the blood within the arterial lumen, allowing it to form complexes with coagulation factors VII/VIIa. This process initiates the coagulation cascade, leading to thrombogenesis. Disrupted plaques can lead either to mural or occlusive thrombosis, causing partial or full blockage, respectively. Mural thrombosis causes ischemic symptoms, such as unstable angina, whereas occlusive thrombosis leads to acute coronary events, such as MI and stroke. Cytokines are involved in all stages of atherosclerosis and have a profound influence on its pathogenesis (see Ramji et al. Cytokine Growth Factor Rev 2015; 26:673-685, which is hereby incorporated by reference in its entirety). In addition to being secondary to atherosclerosis, thrombosis can also be activated in the absence of plaque formation, for example, as a consequence of atrial fibrillation or by direct activation of the clotting cascade.

3. Oxidative Stress

Studies in patients with elevated Hcy levels showed that treated and, especially, untreated patients were susceptible to oxidative stress, as evidenced by altered biomarkers reflecting lipid, protein and DNA oxidative damage in various tissues (see Vanzin et al. Mol Genet Metab 2011; 104:112-117; Vanzin et al. Gene 2014; 539:270-274; Vanzin et al. Cell Mol Neurobiol 2015; 35:899-911, each of which is hereby incorporated by reference in its entirety). Oxidative stress, defined as an imbalance in redox homeostasis, plays a key role in such vascular pathologies as atherosclerosis and its associated thrombosis, where oxidative modification of low-density lipoproteins, endothelial activation and initiation of vascular inflammatory responses are implicated (see Nowak et al. Arterioscler Thromb Vasc Biol 2017; 37: e41-e52, which is hereby incorporated by reference in its entirety). Oxidative stress can be caused by increased levels of ROS (e.g. superoxide (O²⁻) and hydroxyl (HO⁻) radicals and hydrogen peroxide (H₂O₂)) and/or by decreased levels of tissue antioxidants (e.g. superoxide dismutase, catalase and glutathione peroxidase) (see Faverzani et al. Cell Mol Neurobiol 2017; Nowak et al. Arterioscler Thromb Vasc Biol 2017; 37: e41-e52, both of which are hereby incorporated by reference in their entireties). In healthy individuals, ROS are produced as byproducts of normal oxidative metabolism. However, in addition, ROS generation is triggered by such CV risk factors as cigarette smoke, alcohol consumption, hypercholesterolemia, hypertension, diabetes and elevated Hcy levels.

At a molecular level, there are numerous ways that Hcy could cause increased oxidative stress, some discussed previously. For example, accumulation of immunogenic homocysteinylated proteins in the vascular wall could promote inflammation and, consequently, ROS (O²⁻) generation by activated phagocytes). Another potential mechanism is Hcy-induced activation of NMDA receptors, triggering signaling pathways leading to ROS generation. In cardiac microvascular ECs, Hcy induced increased levels of NADPH oxidase, cell surface enzymes that, especially in activated cells, produce high levels of O²⁻. A recent study (see Chen et al. Sci Rep. 2017 Jul. 31; 7(1):6932, which is hereby incorporated by reference in its entirety) suggested that, in the ischemic rat brain, Hcy induced mitochondrial dysfunction, with the expected result of increased ROS production. Hcy is also believed to decrease bioavailability of the beneficial vasodilator NO. O²⁻ reacts with NO to yield the reactive nitrogen species peroxynitrite and, indeed, an Hcy induced increase in tyrosine nitration, an indicator of peroxynitrite-induced protein damage, has also been reported (see Tyagi et al. Vasc Health Risk Manag 2011; 7:31-35, which is hereby incorporated by reference in its entirety). More broadly, thiol-thiol interactions involving Hcy would be expected to perturb cellular redox status, for example, potentially decreasing availability of reduced glutathione and even impairing protein assembly and folding.

In a study of HCU patients before and after treatment, treatment with pyridoxine, folate, betaine and vitamin B₁₂ supplements attenuated lipid oxidative damage in patients but did not change sulfhydryl content or total antioxidant status, both indicators of tissue antioxidant capacity. Nonetheless, there was a significant negative correlation between sulfhydryl group content and Hcy levels, and a positive correlation between levels of the lipid peroxidation product malondialdehyde and those of Hcy. This suggested a potential mechanistic role for Hcy in the oxidative damage observed in HCU (see Vanzin et al. Mol Genet Metab 2011; 104:112-117, which is hereby incorporated by reference in its entirety). Altered lipid profiles, in particular decreased levels of high-density lipoprotein and enrichment of proinflammatory lipid species, were observed in the plasma of untreated and treated HCU patients (see Vanzin et al. Cell Mol Neurobiol 2015; 35:899-911, which is hereby incorporated by reference in its entirety). In another study, significantly more DNA damage was reported in HCU patients than in healthy individuals (see Vanzin et al. Gene 2014; 539:270-274, which is hereby incorporated by reference in its entirety). Together, these findings implicate oxidative stress in the pathogenesis of vascular damage associated with elevated Hcy levels. No correlation was found between Met levels and any oxidative stress-associated parameters, suggesting that Met and its derivatives contribute little to the oxidative damage in HCU (see Vanzin et al. Mol Genet Metab 2011; 104:112-117, which is hereby incorporated by reference in its entirety).

Among a plethora of other molecular effects, oxidative stress is associated with activation of NF-ηB, a group of transcription factors regulating expression of proinflammatory genes, such as cytokines, known to be involved in initiation and progression of atherosclerosis and thrombosis (see Rodriguez-Ayala et al. Atherosclerosis 2005; 180:333-340, which is hereby incorporated by reference in its entirety). In vitro studies showed that treatment of ECs with Hcy activated NF-kβ via ROS production (see van Guldener et al. Curr Hypertens Rep 2003; 5:26-31, which is hereby incorporated by reference in its entirety). Besides modulating gene expression, chemical modification of cellular macromolecules by oxidative stress can directly impact the structure and function of the vasculature and have other localized or systemic effects, as discussed in the rest of the section.

4. Changes in the Vascular Wall

Endothelial dysfunction is generally defined as an imbalance between endothelial-associated factors modulating vascular contractility and relaxation. Among these factors, NO or “endothelial derived relaxation factor” is the most well-known, while hydrogen sulfide (H₂S) is another described more recently (see Jiang et al. Arterioscler Thromb Vasc Biol 2005; 25:2515-2521, which is hereby incorporated by reference in its entirety). Several in vitro studies examined effects of Hcy on endothelial function, albeit using very high levels of Hcy (see Jiang et al. Arterioscler Thromb Vasc Biol 2005; 25:2515-2521; Hossain et al. J Biol Chem 2003; 278:30317-30327; Cai et al. Blood 2000; 96:2140-2148; Zhang et al. J Biol Chem 2001; 276:35867-35874; each of which is hereby incorporated by reference in its entirety). One such study reported an unfolded protein response and programmed cell death in human umbilical vein endothelial cells (HUVEC) treated with Hcy, though Hcy concentrations were several-fold higher than those observed in patients with severely elevated Hcy levels (see Zhang et al. J Biol Chem 2001; 276:35867-35874, which is hereby incorporated by reference in its entirety). In other reports, Cth gamma-lyase (CGL), an enzyme involved in Cth metabolism, generated excess H₂S in patients with elevated Hcy levels (see Papapetropoulos et al. Proc Natl Acad Sci USA 2009; 106:21972-21977; Szabo C et al. Br J Pharmacol 2011; 164:853-865; Chiku et al. J Biol Chem 2009; 284:11601-11612, each of which is hereby incorporated by reference in its entirety). This observation was important because increased H₂S levels were reported to significantly increase collateral vessel growth, capillary density and regional tissue blood flow (see Wang et al. Antioxid Redox Signal 2010; 12:1065-1077, which is hereby incorporated by reference in its entirety). However, high Hcy levels (0.002 to 2 mM) did not significantly affect EC proliferation or phospho-eNOS levels in vitro (see Saha et al. Cystathionine beta-synthase regulates endothelial function via protein S-sulfhydration. FASEB J 2016; 30:441-456, which is hereby incorporated by reference in its entirety). Overall, these results suggest a possible role for Hcy in endothelial dysfunction, though it is not yet clear whether Hcy directly affects ECs in vivo due to the high Hcy levels tested in vitro.

In a recent pharmacological and genetic study, loss of CBS function in ECs was associated with a 50% decrease in cellular H₂S and a 400% decrease in glutathione, with a concomitant increase in cellular ROS levels (see Saha et al. FASEB J 2016; 30:441-456, which is hereby incorporated by reference in its entirety). Silencing CBS in ECs compromised phenotypic and signaling responses to vascular endothelial growth factor (VEGF) and this effect was exacerbated by decreased transcription of vascular endothelial growth factor receptor-2 (VEGFR-2) and neuropilin-1 (NRP-1), primary receptors regulating endothelial functions such as angiogenesis. Transcriptional downregulation of VEGFR-2 and NRP-1 was mediated by decreased stability of transcription factor specificity protein 1 (Sp1), a sulfhydration target of H₂S. Replenishing H₂S, but not glutathione, in CBS-silenced ECs restored Sp1 levels and Sp1 binding to the VEGFR-2 promoter, as well as increasing VEGFR-2 and NRP-1 expression and VEGF-dependent cell proliferation and migration. This suggests that CBS-mediated protein 5-sulfhydration is important for maintaining vascular health and function, supporting previous observations that patients with HCU exhibited endothelial dysfunction(see Celermajer et al. J Am Coll Cardiol 1993; 22:854-858; Rubba et al. Metabolism 1990; 39:1191-1195, both of which are hereby incorporated by reference in their entireties) and raises the possibility that HCU patients suffer from an additional and distinct type of vascular damage, in addition to the type of vascular damage observed in individuals with Hcy levels elevated by other causes.

Dysregulated endothelial function, or other effects on vascular contractility, can lead to blood pressure (BP) abnormalities. Plasma Hcy level was directly linked to BP and Hcy lowering, using folic acid, was associated with decreased BP (see Tripathi P. Molecular and biochemical aspects of homocysteine in cardiovascular diseases. International Cardiovascular Forum J 2016; 6:13; Hackam et al. JAMA 2003; 290:932-940, both of which are hereby incorporated by reference in its entirety). While mechanisms leading to these effects were unclear, Hcy levels are more strongly associated with systolic than diastolic BP. This suggests that elevated Hcy levels increase arterial stiffness. The degree of arterial stiffness is largely determined by the number and function of smooth muscle cells (SMC), the collagen:elastin ratio in the ECM, the quality of collagen and endothelial function (see Tripathi P. Molecular and biochemical aspects of homocysteine in cardiovascular diseases. International Cardiovascular Forum J 2016; 6:13, which is hereby incorporated by reference in its entirety).

Potentially, high levels of Hcy are associated with increased arterial stiffness because of increased SMC proliferation, collagen production and elastin fiber formation (see van Guldener et al. Curr Hypertens Rep 2003; 5:26-31, which is hereby incorporated by reference in its entirety). However, it is also possible that Hcy decreases arterial stiffness by impairing collagen crosslinking. In a study conducted in minipigs, diet-induced Hcy elevation led to “mega artery syndrome” with hyperpulsatile arteries, systolic (but not diastolic) hypertension and extended reactive hyperemia of conduit arteries with dilation of the aorta (see van Guldener et al. Homocysteine and blood pressure. Curr Hypertens Rep 2003; 5:26-31, which is hereby incorporated by reference in its entirety). There was also fragmentation of the arterial wall elastic lamina, correlated with aortic stiffness.

Consistent with these findings, a study conducted in mice, with and without HCU, found that the cross-sectional area of the vessel wall was significantly greater in CBS+/−mice fed a control diet (437±22 μM²) and CBS+/+ (442±36 μm²) and CBS+/− (471±46 μm²) mice fed a high-Met diet, compared with in CBS+/+ (324±18 μm²) mice fed a control diet (p<0.05) (see Baumbach et al. Structure of cerebral arterioles in cystathionine beta-synthase-deficient mice. Circ Res 2002; 91:931-937, which is hereby incorporated by reference in its entirety).

During maximal vasodilation, the stress—strain curves in cerebral arterioles of CBS+/− mice on the control diet and CBS+/+ and CBS+/− mice on the high-Met diet were shifted to the right of the curve for CBS+/+ mice on the control diet. This indicated that cerebral arteriole distensibility was greater in mice with elevated plasma tHcy levels. These results suggest that elevated Hcy levels induced cerebral vascular hypertrophy and altered cerebral vascular mechanics, both effects potentially contributing to an increased incidence of thrombosis, for example, stroke, even in the absence of atherosclerosis (see Baumbach et al. Circ Res 2002; 91:931-937, which is hereby incorporated by reference in its entirety).

Further support for effects of elevated Hcy on vasodilation derives from a study in 5 Italian patients with HCU and tHcy levels ranging from 193.6 to 342 μmol/L (see Evangelisti et al. Int J Cardiol 2009; 134:251-254, which is hereby incorporated by reference in its entirety). Patients showed signs of mild heart valve prolapse and/or regurgitation and connective tissue manifestation.

5. Thrombosis

Elevated Hcy levels are associated with higher risk of deep vein thrombosis, cerebral sinus thrombosis and retinal vein thrombosis (see Spence J D. Lancet Neurol. 2007 September; 6(9):830-8, which is hereby incorporated by reference in its entirety) though multiple studies failed to find an association with risk of MI. Consistent with these results, additional though small studies suggest that HCU is associated with thrombosis, but not necessarily atherosclerosis. Vascular imaging of patients with familial hypercholesterolemia (FH) and HCU showed that, while FH patients exhibited diffuse and focal thickening of carotid arteries and endothelial dysfunction leading to reduced blood flow, HCU patients rarely had plaques in their carotid arteries and were similar to healthy control subjects with regard to both intima-media thickness (IMT) and blood flow velocity in the middle cerebral artery (see Rubba et al. Stroke 1994; 25:943-950, which is hereby incorporated by reference in its entirety). This study suggests that typical atherosclerotic lesions may not be required to precede thrombotic events in HCU and that medial damage leading to thrombosis may also be caused by arterial dilatations.

Support for this observation derives from a study comparing the prevalence of carotid and femoral atherosclerosis (determined by IMT and ankle-brachial index) in 13 patients with enzymatically-proven heterozygous HCU, compared with in 12 healthy subjects with normal Met-loading test results (see de Valk et al. Stroke 1996; 27:1134-1136, which is hereby incorporated by reference in its entirety). No significant differences were observed between groups in mean IMT values, IMT frequency distribution or IMT in each of five arterial segments. These results might be explained by the fact that heterozygous individuals were too young (all <50 years old) to develop structural vascular changes. However, these data also suggest that elevated Hcy levels may primarily affect the coagulation cascade, at least in younger patients. Indeed, a case report of three unrelated HCU patients found that one patient experienced stroke due to intraluminal thrombosis and another patient experienced cardiac or arterial thromboembolism, also without evidence of craniocervical atherosclerosis (see Kelly et al. Neurology 2003; 60:275-279, which is hereby incorporated by reference in its entirety).

Consistent with these observations, Hcy addition to HUVEC and CV1 ECs irreversibly inactivated anticoagulants, protein C and thrombomodulin (see Lentz et al. J Clin Invest 1991; 88:1906-1914, which is hereby incorporated by reference in its entirety). Moreover, Hcy addition to cultured human ECs increased procoagulant tissue factor activity in time- and concentration-dependent manners (see Fryer et al. Arterioscler Thromb 1993; 13:1327-1333, which is hereby incorporated by reference in its entirety).

In both studies, Hcy enhanced the coagulation pathways via a mechanism involving its free thiol group. Together, these data support the hypothesis that perturbations in vascular coagulant mechanisms contribute to increased vascular risk in patients with HCU and that this may play an earlier role in HCU patients, before effects of Hcy on atherosclerosis become apparent.

Lowering Plasma Hcy Levels Reduces Risk of Vascular Complications, Particularly Stroke, in HCU Patients and in the General Population

Recent analyses have found a strong link in the general population between elevated tHcy and stroke risk (see Saposnik et al. Stroke 2009; 40:1365-1372; Spence JD. Homocysteine lowering for stroke prevention: Unravelling the complexity of the evidence. Int J Stroke. 2016 October; 11(7):744-7; Hankey et al. Lancet Neurol 2012; 11:512-520, each of which is hereby incorporated by reference in its entirety).

In the past, the benefits of Hcy-lowering interventions in patients with elevated Hcy levels appeared mixed, with some studies showing a reduction in vascular risk (see Yap et al. J Inherit Metab Dis 1998; 21:738-747; Yap et al. Arterioscler Thromb Vasc Biol 2001; 21:2080-2085; Wilcken et al. J Inherit Metab Dis 1997; 20:295-300; Yap et al. Semin Thromb Hemost 2000; 26:335-340; Saposnik et al. Stroke 2009; 40:1365-1372; Huo et al. JAMA 2015; 313:1325-1335; Hankey et al. Lancet Neurol 2012; 11:512-520, each of which is hereby incorporated by reference in its entirety) and others showing no benefit (see Marti-Carvajal et al. Cochrane Database Syst Rev 2015; 1:CD006612; Ebbing et al. JAMA 2008; 300:795-804; Bonaa et al. N Engl J Med 2006; 354:1578-1588; Liem et al. Heart 2005; 91:1213-1214; Toole et al. JAMA 2004; 291:565-575; B vitamins in patients with recent transient ischemic attack or stroke in the VITAmins TO Prevent Stroke (VITATOPS) trial: a randomized, double-blind, parallel, placebo-controlled trial (see Lancet Neurol 2010; 9:855-865; Albert et al. JAMA 2008; 299:2027-2036, each of which is hereby incorporated by reference in its entirety). Many of these trials tested vitamin interventions, primarily B vitamin (B₆ and B₁₂) and folate supplementation, for Hcy lowering. Consequently, study outcomes were subject to confounding factors, such as whether subjects lived in regions where folate fortification is practiced, had renal dysfunction making them more vulnerable to cobalamin toxicity, had vitamin B₁₂ deficiencies related to poor absorption, which is relatively common in the elderly, or were on antiplatelet medication. These recent analyses considered these confounding factors, and concluded that, when such key variables are accounted for, elevated tHcy increases the risk of stroke in the general population (see Mudd et al. Arterioscler Thromb Vasc Biol 2000; 20:1704-1706; Spence J D. Int J Stroke. 2016 October; 11(7):744-7; Spence J D, Clin Chem Lab Med. 2013 Mar 1; 51(3):633-7, each of which is hereby incorporated by reference in its entirety).

Hcy-lowering therapy reduces risk of stroke, even in individuals without HCU. In the HOPE-2 (Heart Outcomes Prevention Evaluation 2) trial, 5,522 adults aged ≥55 years, with a history of vascular disease or diabetes mellitus and at least one additional CV risk factor, were randomized to either vitamin supplementation (folic acid, vitamin B₆ and vitamin B₁₂) or placebo for 5 years (see Saposnik et al. Stroke 2009; 40:1365-1372, which is hereby incorporated by reference in its entirety). Mean baseline Hcy concentrations were 11.5 μmol/L in both groups and patients taking a daily vitamin supplement containing >0.2 mg folic acid at baseline were excluded from the study. Overall, Hcy-lowering (mean 3.0 μmol/L vs placebo) was associated with a significant 27% relative risk reduction (1.3% absolute reduction) in stroke (HR, 0.75; 95% CI, 0.59 to 0.97) and nonsignificant reductions in ischemic stroke (HR, 0.81; 95% CI, 0.60 to 1.09) and hemorrhagic stroke (HR, 0.80; 95% CI, 0.32 to 2.02). In subgroup analyses, the relative risk of stroke was most reduced among patients with baseline Hcy levels in the highest quartile (4.3% absolute risk reduction). Treatment benefit was greatest in patients aged <69 years, those from regions without folic acid food fortification and those not receiving antiplatelet or lipid-lowering drugs at enrollment. Thus, the HOPE-2 trial reported a reduced incidence rate of stroke in individuals receiving B vitamins versus placebo (hazard ratio (HR) 0.75; 95% CI, 0.59 to 0.97), whereas the risk of MI was similar in both treatment groups (RR 0.98; 95% CI, 0.85 to 1.14) (see Saposnik et al. Stroke 2009; 40:1365-1372; Lonn E, Yusuf S, Arnold M J et al. Homocysteine lowering with folic acid and B vitamins in vascular disease. N Engl J Med 2006; 354:1567-1577, both of which are hereby incorporated by reference in its entirety).

Consistent with results from the HOPE-2 trial, a sub-analysis of the VITATOPS trial, in which 8,164 patients with recent stroke or transient ischemic attack were randomized to double-blind treatment with B vitamins or placebo for a median 3.4 years, found that B vitamins significantly reduced the primary composite outcome (stroke, MI or death from vascular causes) among patients not taking antiplatelet therapy at baseline (17 versus 21% with placebo; HR 0.76, 0.60 to 0.96). No significant effect of B vitamins was observed in individuals receiving antiplatelet therapy (see Hankey et al. Antiplatelet therapy and the effects of B vitamins in patients with previous stroke or transient ischemic attack: a post-hoc sub-analysis of VITATOPS, a randomized, placebo-controlled trial. Lancet Neurol 2012; 11:512-520, which is hereby incorporated by reference in its entirety). In this study, tHcy levels were significantly decreased from 12.4 to 13.7 μmol/L at baseline to 9.9 to 10.5 μmol/L after vitamin therapy, irrespective of whether patients received antiplatelet therapy (p<0.0001 for both treatment groups) (see Hankey et al. Lancet Neurol 2012; 11:512-520, which is hereby incorporated by reference in its entirety).

Inflammatory cascades are believed to contribute to ischemic stroke pathogenesis. A report from the Framingham Offspring Study in 3,224 participants (see Shoamanesh et al. Neurology. 2016 September; 87(12):1206-11, which is hereby incorporated by reference in its entirety) found that elevated levels of tHcy and three other inflammatory markers were strongly associated with the risk of ischemic stroke and improved the predictive ability of the Framingham Stroke Risk Profile score.

A meta-analysis of data from the VISP and VITATOPS studies found that patients with normal renal function not previously exposed to high dose cyanocobalamin benefited significantly from vitamin therapy including high dose cyanocobalamin (0.78, 0.67 to 0.90; interaction p=0.03) whereas vitamin therapy including high dose cyanocobalamin (a form of vitamin B) had no effect on stroke risk in individuals with impaired renal function (RR 1.04, 95% CI 0.84 to 1.27) (see Spence J D. Lancet Neurol. 2007 September; 6(9):830-8, which is hereby incorporated by reference in its entirety). These results suggested potentially confounding effects of cyanocobalamin, known to be nephrotoxic, associated with cyanide accumulation, in patients with significantly impaired renal function (see Spence J D, Clin Chem Lab Med. 2013 Mar. 1; 51(3):633-7, which is hereby incorporated by reference in its entirety). Consistent with this, in the DIVINe trial (Diabetic Intervention with Vitamins in Nephropathy), high doses B vitamins, including cyanocobalamin at 1000 μg, were harmful, exacerbating eGFR decline (see Spence J D. Int J Stroke. 2016 October; 11(7):744-7; House et al. JAMA 2010; 303: 1603-1609, both of which are hereby incorporated by reference in their entireties). Together, such findings support the use of non-cyanide containing B vitamins, such as methylcobalamin, instead of cyanocobalamin, to lower Hcy levels in individuals at high risk of stroke, especially those with renal insufficiency.

Cyanocobalamin and cyanide toxicity were further implicated in previous trials as confounding factors in the presence of renal impairment by results from the CSPPT (China Stroke Primary Prevention Trial (see Huo et al. JAMA. 2015 Apr 7; 313(13):1325-35, which is hereby incorporated by reference in its entirety). The treatment benefit demonstrated with renal impairment in the CSPPT was counter to the lack of benefit observed in the DIVINe, VISP and VITOPS trials, likely due to cyanocobalamin treatment in the latter trials.

Because in China folate fortification has not yet been implemented, the effect of folic acid supplementation on lowering tHcy levels could be studied there in a large population. The CSPPT was a randomized double-blind trial conducted in 20,702 adults with hypertension but no history of stroke or MI, which demonstrated that folic acid significantly decreased the risk of first stroke (2.7 vs 3.4% without folic acid, HR 0.79; 95% CI 0.68 to 0.93), first ischemic stroke (2.2 vs 2.8% without folic acid, HR 0.76; 95% CI 0.64 to 0.91) and composite CV events (CV death, MI and stroke; 3.1% vs 3.9% without folic acid, HR 0.80; 95% CI 0.69 to 0.92). There were, in contrast, no significant differences between the two groups in risks of hemorrhagic stroke, all-cause deaths or frequencies of AEs. In a sub-study of the CSPPT (see Xu et al. JAMA Intern Med. 2016 Oct. 1; 176(10):1443-1450, which is hereby incorporated by reference in its entirety), both subjects with or without renal impairment (eGFR below 60 mL/min/1.73 m²) benefited from folic acid, and the sub-study furthermore confirmed that the group treated with folic acid had a much greater drop in serum Hcy than did the group not receiving folic acid (1.9 vs 0.2 μmol/L, respectively, p<0.001).

A 2017 Cochrane review (see Marti-Carvajal et al. Cochrane Database Syst Rev. 2017 Aug. 17; 8:CD006612, which is hereby incorporated by reference in its entirety), analyzing 15 randomized controlled trials involving 74,422 participants, reported a small difference in effect of Hcy-lowering with B vitamins on stroke but no effects on MI, death from any cause or AEs. Compared with placebo/standard care, Hcy lowering interventions were associated with a decreased incidence of nonfatal or fatal stroke (4.33% vs 5.1% for control; RR 0.90, 95% CI 0.82 to 0.99), but had no effect on the incidence of nonfatal or fatal MI (7.1% vs 6.0% for placebo; relative risk (RR) 1.02, 95% CI 0.95 to 1.10) or death from any cause (11.7% vs 12.3% for placebo; RR 1.01, 95% CI 0.96 to 1.06) in the general population. This review was an update of three earlier versions (2009, 2013 and 2015), which had previously concluded that there was no evidence supporting effects of Hcy-lowering interventions on CV events, though the 2015 review had indicated a non-significant trend in reduced incidence of stroke (see Marti-Carvajal Cochrane Database Syst Rev 2015; 1:CD006612, which is hereby incorporated by reference in its entirety). The strength of the evidence on Hcy lowering interventions on stroke has become stronger as additional trials have become available.

The studies included in the review used various regimens of vitamin supplementation as Hcy-lowering therapy (see Marti-Carvajal et al. Cochrane Database Syst Rev. 2017 Aug. 17; 8:CD006612, which is hereby incorporated by reference in its entirety). The 2017 review added three new trials to those in the 2015 review. Of the total, 10 trials used pyridoxine plus vitamins B₉ (folate) and B₁₂, five used only vitamin B9 and one of the 10 used 5-methyltetrahydrofolate instead of folic acid. Some trials included concomitant drugs (in both control and vitamin treated groups), 7 trials with lipid-lowering and one with anti-hypertensive agents. Overall, the Hcy-lowering treatments resulted in a relatively small decrease in mean tHcy levels. Moreover, three studies were performed in a folic acid-fortified population and two in a mixed population (some subjects receiving a fortified diet and others not), which may have masked Hcy-lowering effects. Considering the confounding factors that might influence such studies, it is not a surprise that the earlier reviews did not identify any significant CV effects of the vitamin treatment regimens and that only modest effects on stroke were reported in the newest review. It is also notable that the many potential causes of elevated Hcy levels were not examined in the studies included in the Cochrane review (see Marti-Carvajal et al. Cochrane Database Syst Rev 2015; 1:CD006612, which is hereby incorporated by reference in its entirety). There may be additional factors affecting the pathophysiology of vascular risk in patients with HCU and at younger ages than in the general population. It is noteworthy that even modest decreases in Hcy levels are significantly associated with reductions in stroke risk.

An interesting question arising from several of these analyses is why some studies indicated an effect of Hcy levels on stroke but not on MI. Relevant to this question, Spence (see Spence J D. Lancet Neurol. 2007 September; 6(9):830-8, which is hereby incorporated by reference in its entirety) pointed out key differences between MI and cerebral infarction, with cerebral infarction being closely associated to thrombosis/embolic events but in situ thrombosis being secondary to plaque rupture in a coronary artery in nearly all MI events. Thus, Spence concluded that a substantial proportion of strokes are related to thrombotic processes, which can be associated with raised tHcy.

Elevated tHcy levels may be important not only in cardioembolic stroke but also atheroembolic and lacunar infarction. A study in elderly patients with atrial fibrillation treated with anticoagulant therapy found that high levels of tHcy (>90^{th} percentile) were associated with a 4.7-fold increase in ischemic complications (see Poli et al. J Am Coll Cardiol 2009; 54:999-1002, which is hereby incorporated by reference in its entirety). Another study in patients with cryptogenic ischemic stroke found that those with patent foramen ovale (a risk factor for cerebral infarction) had significantly higher plasma tHcy levels than those without (8.9±3 versus 7.9±2.6 μmol/L respectively; p=0.021) (see Ozdemir et al. J Neurol Sci 2008; 275:121-127, which is hereby incorporated by reference in its entirety). In reviewing such findings, Spence (see Spence J D. Homocysteine lowering for stroke prevention: Unravelling the complexity of the evidence. Int J Stroke. 2016 October; 11(7):744-7, which is hereby incorporated by reference in its entirety) suggested that Hcy affects primarily the formation of red thrombus (a fibrin polymer mesh with entrapped red blood cells, that forms in the setting of stasis) but that lacunar infarction and carotid plaques are also significantly related to tHcy as levels of tHcy were also significantly higher in patients with microemboli on transcranial Doppler (16.2 vs. 10.1 mmol/L) and most such microemboli are thought to be platelet aggregates, reduced by dual antiplatelet therapy (see Spence J D. Homocysteine lowering for stroke prevention: Unravelling the complexity of the evidence. Int J Stroke. 2016 October; 11(7):744-7, which is hereby incorporated by reference in its entirety).

A study conducted in 32 HCU patients (aged between 9 and 66 years) treated with pyridoxine, folic acid and hydroxocobalamin for a total of 539 patient-years, reported two vascular events (one fatal pulmonary embolus and one MI) during treatment (see Wilcken et al. J Inherit Metab Dis 1997; 20:295-300, which is hereby incorporated by reference in its entirety). According to the epidemiological study by Mudd et al., 21 events would have been expected over the same period of time without treatment (RR 0.09 (95% CI 0.02 to 0.38); p=0.0001). A second study, conducted in 84 patients, from three countries, aged between 2.5 and 70 years reported five cases of venous embolism (VE) during 1314 patient-years of treatment, one pulmonary embolism, two MIs, and two abdominal aneurysms (see Yap et al. Semin Thromb Hemost 2000; 26:335-340, which is hereby incorporated by reference in its entirety). According to Mudd et al., (see Mudd et al. Am J Hum Genet 1985; 37:1-31, which is hereby incorporated by reference in its entirety) 53 cases of VE would have been expected in untreated patients (RR 0.091 (95% CI 0.043 to 0.190); p<0.001).

Supportive evidence for these results derives from a large international multicenter observational study of 158 patients with HCU (see Yap et al. Arterioscler Thromb Vasc Biol 2001; 21:2080-2085, which is hereby incorporated by reference in its entirety), the majority ranging from 10 to 30 years of age. In this study 17 vascular events were observed among 12 treated individuals, three cases of pulmonary embolism, two MIs, five cases of deep venous thrombosis, three cerebrovascular accidents, one transient ischemic attack, one sagittal sinus thrombosis and two abdominal aortic aneurysms. Without treatment, 112 vascular events would have been expected in a similar population (RR 0.09 (95% CI 0.036 to 0.228); p <0.0001). This study also highlights how even treated and extensively followed young adult HCU patients suffer from poor clinical outcomes compared with the general population.

An elevated plasma tHcy level is a risk factor for vascular disease and a strong predictor of mortality in patients with CAD, both with and without CBSD (see Mudd et al. Am J Hum Genet 1985; 37:1-31; Karaca et al. Gene 2014; 534:197-203; Kelly et al. Neurology 2003; 60:275-279; Faeh et al. Swiss Med Wkly 2006; 136:745-756; Boushey et al. JAMA 1995; 274:1049-1057; Clarke et al. JAMA 2002; 288:2015-2022; Hankey et al. Lancet 1999; 354:407-413; Khan et al. Stroke 2008; 39:2943-2949; Graham et al. JAMA 1997; 277:1775-1781; Clarke et al. N Engl J Med 1991; 324:1149-1155; Clarke et al. Ir J Med Sci 1992; 161:61-65; Woodward et al. Blood Coagul Fibrinolysis 2006; 17:1-5; Refsum et al. Annu Rev Med 1998; 49:31-62; Yoo et al. Stroke 1998; 29:2478-2483; Selsun et al. N Engl J Med 1995; 332:286-291; Wald et al. BMJ 2002; 325:1202; Bautista et al. J Clin Epidemiol 2002; 55:882-887; Brattstrom et al. Atherosclerosis 1990; 81:51-60; Lussana et al. Thromb Res 2013; 132:681-684; Casas et al. Lancet 2005; 365:224-232; McCully KS. Am J Pathol 1969; 56:111-128; Magner et al. J Inherit Metab Dis 2011; 34:33-37; Wilcken et al. J Clin Invest 1976; 57:1079-1082; Nygard et al. N Engl J Med 1997; 337:230-236, each of which is hereby incorporated by reference in its entirety).

Of the evidence for a causal relationship between tHcy levels and CV risk (see Boushey et al. JAMA 1995; 274:1049-1057, which is hereby incorporated by reference in its entirety), the strongest relationships were demonstrated between tHcy and stroke or PAD, (see Clarke et al. JAMA 2002; 288:2015-2022; Khan et al. Stroke 2008; 39:2943-2949; Wald et al. BMJ 2002; 325:1202; Casas et al. Lancet 2005; 365:224-232; Brattstrom et al. Haemostasis 1989; 19 Suppl 1:35-44, each of which is hereby incorporated by reference in its entirety) including in patients with both homozygous and heterozygous CBS mutations (see Rubba et al. Metabolism 1990; 39:1191-1195, which is hereby incorporated by reference in its entirety).

Mechanisms potentially linking elevated Hcy to vascular damage are varied and complex. Several studies implicated oxidative stress (see Faverzani et al. Cell Mol Neurobiol 2017; Nowak et al. Arterioscler Thromb Vasc Biol 2017; 37: e41-e52; Vanzin et al. Mol Genet Metab 2011; 104:112-117; Vanzin et al. Gene 2014; 539:270-274; Vanzin et al. Lipid, Cell Mol Neurobiol 2015; 35:899-911, each of which is hereby incorporated by reference in its entirety), including by inflammatory/immune activation via NF-ηB (see Rodriguez-Ayala et al. Atherosclerosis 2005; 180:333-340; van Guldener et al. Curr Hypertens Rep 2003; 5:26-31, both of which are hereby incorporated by reference in its entirety). While such inflammatory processes are characteristic of atherosclerosis, typical atherosclerotic lesions do not need to precede thrombotic events in patients with HCU (see Rubba et al. Stroke 1994; 25:943-950; de Valk et al. Stroke 1996; 27:1134-1136, both of which are hereby incorporated by reference in its entirety). Instead, medial damage leading to thrombosis is believed to be caused by Hcy-mediated endothelial dysfunction (see Marti-Carvajal et al. Cochrane Database Syst Rev 2015; 1:CD006612; Celermajer et al. J Am Coll Cardiol 1993; 22:854-858; Rubba et al. Metabolism 1990; 39:1191-1195; Jiang et al. Arterioscler Thromb Vasc Biol 2005; 25:2515-2521; Hossain et al. J Biol Chem 2003; 278:30317-30327; Cai et al. Blood 2000; 96:2140-2148; Zhang et al. J Biol Chem 2001; 276:35867-35874; Papapetropoulos et al. Proc Natl Acad Sci USA 2009; 106:21972-21977; Szabo et al. Br J Pharmacol 2011; 164:853-865; Chiku et al. J Biol Chem 2009; 284:11601-11612; Wang et al. Antioxid Redox Signal 2010; 12:1065-1077; Saha S, et al. FASEB J 2016; 30:441-456; Ebbing et al. JAMA 2008; 300:795-804; Bonaa et al. N Engl J Med 2006; 354:1578-1588, each of which is hereby incorporated by reference in its entirety) enhanced coagulation pathways (see Spence J D. Int J Stroke. 2016 October; 11(7):744-7; Fryer et al. Arterioscler Thromb 1993; 13:1327-1333; Lentz et al. J Clin Invest 1991; 88:1906-1914, each of which is hereby incorporated by reference in its entirety) and increased vascular dilatation, similar to thrombotic processes implicated in Marfan patients (see Kelly et al. Neurology 2003; 60:275-279; Tripathi P. Molecular and biochemical aspects of homocysteine in cardiovascular diseases. International Cardiovascular Forum J 2016; 6:13; van Guldener et al. Curr Hypertens Rep 2003; 5:26-31; Hackam et al. JAMA 2003; 290:932-940; Baumbach et al. Circ Res 2002; 91:931-937; Evangelisti et al. Int J Cardiol 2009; 134:251-254; de Valk et al. Stroke 1996; 27:1134-1136, each of which is hereby incorporated by reference in its entirety). Consistent with observations of endothelial dysfunction in patients with HCU, pharmacological and genetic research implicated CBS-mediated protein S-sulfhydration in the maintenance of vascular health and function (see Celermajer et al. J Am Coll Cardiol 1993; 22:854-858; Rubba et al. Metabolism 1990; 39:1191-1195, both of which are hereby incorporated by reference in its entirety). Thus, it is likely that the mechanisms of vascular damage in HCU patients are more varied than in the general population with CV disease.

There is considerable evidence that Hcy-lowering is beneficial against stroke risk in individuals with and without HCU (see Saposnik et al. Stroke 2009; 40:1365-1372; Huo et al. JAMA 2015; 313:1325-1335; Lonn et al. N Engl J Med 2006; 354:1567-1577; Hankey et al. Lancet Neurol 2012; 11:512-520; Spence J D. Lancet Neurol. 2007 September; 6(9):830-8; each of which is hereby incorporated by reference in its entirety). Although mean baseline Hcy levels were relatively low in the studies conducted in the general population, reductions in stroke incidence were significantly correlated with Hcy-lowering interventions, pointing to the benefits of even small decreases in tHcy levels.

Updated reviews of relevant trials support beneficial effects of Hcy-lowering treatments in stroke (see Marti-Carvajal et al. Cochrane Database Syst Rev. 2017 Aug. 17; 8:CD006612, which is hereby incorporated by reference in its entirety). Confounding factors, such as whether there was folate fortification, vitamin B₁₂ deficiency or renal dysfunction leaving patients vulnerable to high dose cyanocobalamin, or whether subjects were on antiplatelet medications, have blurred trial outcomes. Recent analyses demonstrated that, when such factors were accounted for, the link between tHcy and stroke risk was strong in the general population (see Spence J D, Clin Chem Lab Med. 2013 Mar. 1; 51(3):633-7, which is hereby incorporated by reference in its entirety). Importantly, however, in patients with HCU with much higher tHcy levels than the general population, vascular benefits of Hcy lowering have been consistently demonstrated (see Yap et al. J Inherit Metab Dis 1998; 21:738-747; Eslamiyeh et al. Iran J Child Neurol 2015; 9:53-57; Saboul et al. J Child Neurol 2015; 30:107-112; Yap et al. Arterioscler Thromb Vasc Biol 2001; 21:2080-2085; Woods et al. BMJ Case Rep 2017; 2017; Wilcken et al. J Inherit Metab Dis 1997; 20:295-300; Yap et al. Semin Thromb Hemost 2000; 26:335-340; Ruhoy et al. Pediatr Neurol 2014; 50:108-111, each of which is hereby incorporated by reference in its entirety).

These findings suggest that an elevated Hcy level is a risk factor for CV disease, especially stroke, in patients with and without HCU and that CV or cerebrovascular risk can be reduced through long-term Hcy-lowering therapy.

Thromboembolism is the major cause of morbidity and premature death in HCU patients (see Mudd et al. Am J Hum Genet 1985; 37:1-31; Karaca et al. Gene 2014; 534:197-203; Yap S. J Inherit Metab Dis 2003; 26:259-265, each of which is hereby incorporated by reference in its entirety). The overall rate of thromboembolic events in patients with untreated HCU is approximately 10% per year (see Cattaneo M. Semin Thromb Hemost 2006; 32:716-723, which is hereby incorporated by reference in its entirety), with the risk increasing after surgery and during or immediately after pregnancy (see Mudd et al. Am J Hum Genet 1985; 37:1-31; Novy et al. Thromb Haemost 2010; 103:871-873, both of which are hereby incorporated by reference in its entirety). Thromboembolism can affect any blood vessel, but venous thrombosis (in particular cerebral sinovenous thrombosis (CSVT)) is more common than arterial thrombosis in patients with HCU (see Mudd et al. Am J Hum Genet 1985; 37:1-31; Karaca et al. Gene 2014; 534:197-203; Eslamiyeh et al. Iran J Child Neurol 2015; 9:53-57; Saboul et al. J Child Neurol 2015; 30:107-112, each of which is hereby incorporated by reference in its entirety).

A study in 629 untreated HCU patients showed that, of the observed 253 vascular events (occurring in 158 patients), 81 (32%) were cerebrovascular accidents, 130 (51%) affected peripheral veins (with 32 resulting in pulmonary embolism), 10 (4%) led to myocardial infarctions (MI), 28 (11%) affected peripheral arteries and four (2%) fell into none of these categories (see Mudd et al. Am J Hum Genet 1985; 37:1-31, which is hereby incorporated by reference in its entirety). Cerebrovascular accidents, especially CSVT, have been described in infants (see Mahale et al. J Pediatr Neurosci. 2017 April-June; 12(2):206-207, which is hereby incorporated by reference in its entirety), although more typically appear in young adults (see Yap et al. Arterioscler Thromb Vasc Biol 2001; 21:2080-2085, which is hereby incorporated by reference in its entirety). Cerebrovascular events were reported to be only marginally related to the pyridoxine-response category of the patients.

The risk of vascular events was approximately 30% in patients aged <20 years, rising to 50% by the age of 30 years. However, symptoms can occur at any age and fatal thrombosis has been described in infants as young as 6 months (see Cardo et al. Dev Med Child Neurol 1999; 41:132-135, which is hereby incorporated by reference in its entirety). After the age of 10 years, one vascular event is expected per 25 years. In general, the first signs of HCU in children are cognitive symptoms, presenting as developmental delay during the first or second year of life and/or lens dislocation/high myopia. In contrast, adults are more likely to present with vascular events.

D. Effects of Diet on Phenotypic Outcomes

In some embodiments, I278T mice, a mouse model of HCU, were used to evaluate long-term impact of enzyme therapy for HCU with 20NHS PEG-CBS on clinical endpoints relevant to human patients. The efficacy of 20NHS PEG-CBS on a background of normal methionine intake (REG) and a Met-restricted diet (MRD) as well as with MRD alone. Treatment with 20NHS PEG-CBS can result in 90% decrease in plasma homocysteine concentrations and correction of learning/cognition, endothelial dysfunction, hemostasis, bone mineralization, and body composition phenotypes associated with HCU. In certain embodiments, treatment with 20NHS PEG-CBS with a background of the MRD normalized plasma Hcy. The MRD alone has been observed to decrease plasma Hcy by 67% and correct the HCU phenotype in I278T mice. However, the MRD increased anxiety and reduced bone mineral content in both I278T mice and wild type controls. Therefore, 20NHS PEG-CBS is highly efficacious for the treatment for HCU in subjects with a background of a REG or a Met-restricted diet. In fact, ET with 20NHS PEG-CBS on background of normal Met intake performs equally or yields better results compared with a Met-restricted diet.

To summarize, the MRD alone is effective in correcting multiple symptoms of HCU in spite of failing to reduce plasma Hcy concentration below the recommended level and leading to increased anxiety and reduced bone mineralization. On other hand, enzyme therapy with 20NHS PEG-CBS, as described herein, decreased plasma Hcy concentrations below 100 μM and corrected all the monitored symptoms of HCU. Furthermore, 20NHS PEG-CBS retains its efficacy under Met restriction yielding fully normalized plasma biochemical profile. By extrapolating these data to human patients, the results establish 20NHS PEG-CBS as a single life-long therapy could be efficacious in prevention and correction of clinical symptoms of HCU. In addition, treatment with 20NHS PEG-CBS should allow for relaxation of Met/diet restriction, and thus, in turn, substantially improve quality of life of HCU patients and their families.

E. Neurological Complications

Studies have shown that early decreases in Hcy levels, induced by a low Met diet, folic acid/B vitamin supplementation and/or pyridoxine and betaine therapy can prevent and sometimes reverse progression of various neurological disorders and allow normal IQ development in patients with HCU (see El Bashir et al. JIMD Rep 2015; 21:89-95; Yap et al. J Inherit Metab Dis 2001; 24:437-447; Mech A W, Farah A. Correlation of clinical response with homocysteine reduction during therapy with reduced B vitamins in patients with MDD who are positive for MTHFR C677T or A1298C polymorphism: a randomized, double-blind, placebo-controlled study. J Clin Psychiatry 2016; 77:668-671; Grobe H. Eur J Pediatr 1980; 135:199-203; each of which is hereby incorporated by reference in its entirety). Further evidence is provided in case studies in patients with HCU, in which significant decreases, even normalization, of Hcy levels resulted in complete or partial correction of CNS outcomes (see Yap et al. J Inherit Metab Dis 2001; 24:437-447; Brenton et al. J Child Neurol 2014; 29:88-92; Rezazadeh et al. Child Neurol Open 2014; 1:2329048X14545870; Kaeser et al. J Neurol Neurosurg Psychiatry 1969; 32:88-93; Colafrancesco et al. Eur J Pediatr 2015; 174:1263-1266; Yokoi et al. Pediatr Int 2008; 50:694-695; Li et al. Pathology 1999; 31:221-224, each of which is hereby incorporated by reference in its entirety).

Associations between elevated levels of Hcy and CNS symptoms, including cognitive symptoms, neurodegenerative diseases, seizures, dystonia, psychosis, cognitive impairment, dementia, and depression, are well documented in HCU patients and in the general population (see Morris et al. J Inherit Metab Dis 2017; 40:49-74; Abbott et al. Am J Med Genet 1987; 26:959-969; Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327; Hidalgo et al. Eur Child Adolesc Psychiatry 2014; 23:235-238; Smith et al. PLoS One 2010; 5: e12244; Seshadri et al. N Engl J Med 2002; 346:476-483; Bottiglieri et al. J Neurol Neurosurg Psychiatry 2000; 69:228-232; Bjelland et al. Arch Gen Psychiatry 2003; 60:618-626; Tolmunen et al. Am J Clin Nutr 2004; 80:1574-1578; Kaeser et al. J Neurol Neurosurg Psychiatry 1969; 32:88-93; Golimbet et al. Psychiatry Res 2009; 170:168-171; Clarke et al. Arch Neurol 1998; 55:1449-1455; Permoda-Osip et al. Neuropsychobiology 2014; 69:107-111; Oliveira et al. BMJ Case Rep 2016; 2016; Troen et al. Proc Natl Acad Sci USA 2008; 105:12474-12479; Sudduth et al. J Cereb Blood Flow Metab 2013; 33:708-715; Hainsworth et al. Biochim Biophys Acta 2016; 1862:1008-1017; Herrmann et al. Clin Chem Lab Med 2011; 49:435-441; Kim et al. J Nutr 2007; 137:2093-2097; Selhub et al. Am J Clin Nutr 2000; 71:614S-620S; McCaddon et al. Dement Geriatr Cogn Disord 2001; 12:309-313; Smallwood et al. Neuropathol Appl Neurobiol 2012; 38:337-343; Beydoun et al. BMC Public Health 2014; 14:643; Gortz et al. J Neurol Sci 2004; 218:109-114; Health Quality O. Vitamin B12 and cognitive function: an evidence-based analysis. Ont.Health Technol.Assess.Ser.13 (23), 1e45. 2013. Ref Type: Online Source; Salagre et al. Eur Psychiatry 2017; 43:81-91, each of which is hereby incorporated by reference in its entirety). The mechanisms leading to CNS disorders in individuals with elevated Hcy levels are believed to involve tHcy-mediated neuronal damage (see Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327; Hainsworth et al. Biochim Biophys Acta 2016; 1862:1008-1017; Stefanello et al. Metab Brain Dis 2007; 22:172-182; Toborek et al. Atherosclerosis 1995; 115:217-224, each of which is hereby incorporated by reference in its entirety), damage to the vascular endothelium caused by Hcy-mediated oxidative stress (see Vivitsky et al. Am J Physiol Regul IntegrComp Physiol 2004; 287: R39-R46, which is hereby incorporated by reference in its entirety), neuron loss (see Yeganeh et al. J Mol Neurosci 2013; 50:551-557; Heider et al. J Neural Transm Suppl 2004; 1-13, both of which are hereby incorporated by reference in its entirety) and attenuated neural network activity (see Gortz et al. J Neurol Sci 2004; 218:109-114, which is hereby incorporated by reference in its entirety). Depression and convulsions are thought to be caused, at least in part, by Hcy-mediated decreases in cerebral adenosine levels, with subsequent decreases in levels of norepinephrine and dopamine (see Mech et al. J Clin Psychiatry 2016; 77:668-671; Domagala et al. Thromb Res 1997; 87:411-416; Vivitsky et al. Am J Physiol Regul IntegrComp Physiol 2004; 287: R39-R46; Folstein et al. Am J Psychiatry 2007; 164:861-867, each of which is hereby incorporated by reference in its entirety).

These findings demonstrated a strong correlation between Hcy levels and an increased risk of CNS disorders in patients with HCU. Early Hcy-lowering therapy is essential for the normal development of children with early-onset HCU and for the correction or improvement of CNS disorders in patients diagnosed with HCU later in life.

A study in 63 HCU patients found that 51% had psychiatric disorders, including anxiety and episodic depression (10%), chronic behavioral disorders (e.g. aggression and drug or alcohol abuse) (17%), chronic obsessive-compulsive disorder (5%) and personality disorders (19%) (see Abbott et al. Am J Med Genet 1987; 26:959-969, which is hereby incorporated by reference in its entirety). Psychosis may be a presenting sign in adolescence (see Hidalgo et al. Eur Child Adolesc Psychiatry 2014; 23:235-238, which is hereby incorporated by reference in its entirety).

If untreated, approximately 90% of pyridoxine non-responsive patients have learning difficulties (see Mudd et al. Am J Hum Genet 1985; 37:1-31, which is hereby incorporated by reference in its entirety), with IQs typically ranging from 10 to 138, with a mean of 57 in pyridoxine non-responsiveness individuals, compared with 79 in untreated pyridoxine-responsive patients and 105 in treated pyridoxine-responsive patients with good compliance (see Yap et al. J Inherit Metab Dis 2001; 24:437-447, which is hereby incorporated by reference in its entirety). Seizures affect 20% of non-responsive patients by the age of 12 years and several cases were reported of movement disorders unrelated to basal ganglia infarction, including polymyoclonus, dystonia and Parkinson disease (see Morris et al. J Inherit Metab Dis 2017; 40:49-74; Rezazadeh et al. Child Neurol Open 2014; 1:2329048X14545870, both of which are hereby incorporated by reference in its entirety).

An association between HCU and neuropsychiatric symptoms was first described by Schimke et al. in 1965 (see Schimke et al. JAMA 1965; 193:711-719, which is hereby incorporated by reference in its entirety). The association was later supported by a study reporting psychopathology in more than 50% of CBS-deficient patients (see Abbott et al. Am J Med Genet 1987; 26:959-969, which is hereby incorporated by reference in its entirety). Since then, numerous epidemiological studies showed positive, dose-dependent relationships between even mild increases in plasma tHcy and the risk of CNS disorders, including cognitive symptoms and neurodegenerative diseases (see Morris et al. J Inherit Metab Dis 2017; 40:49-74; Seshadri et al. N Engl J Med 2002; 346:476-483; Clarke et al. Arch Neurol 1998; 55:1449-1455; Hainsworth et al. Biochim Biophys Acta 2016; 1862:1008-1017; Herrmann et al. Clin Chem Lab Med 2011; 49:435-441; Kim J et al. J Nutr 2007; 137:2093-2097; Selhub et al. Am J Clin Nutr 2000; 71:614S-620S; McCaddon et al. Dement Geriatr Cogn Disord 2001; 12:309-313; Smallwood et al. Neuropathol Appl Neurobiol 2012; 38:337-343; Beydoun et al. BMC Public Health 2014; 14:643; each of which is hereby incorporated by reference in its entirety). In general, patients with severely elevated Hcy levels (50 to 200 μM/L) tend to present with acute neuronal dysfunction, including seizures and psychosis, whereas more moderate Hcy levels (15 to 50 μM/L) are associated with cognitive impairment and dementia (see Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327; Gortz et al. J Neurol Sci 2004; 218:109-114, both of which are hereby incorporated by reference in its entirety).

An elevated Hcy level is widely accepted as a robust and independent risk factor for cognitive impairment (see Smith et al. PLoS One 2010; 5: e12244; Seshadri et al. N Engl J Med 2002; 346:476-483, both of which are hereby incorporated by reference in their entireties) onset of dementia (see Health Quality O. Vitamin B12 and cognitive function: an evidence-based analysis. Ont.Health Technol.Assess.Ser.13 (23), 1e45. 2013. Ref Type: Online Source, which is hereby incorporated by reference in its entirety) and Alzheimer's disease (see Seshadri et al. N Engl J Med 2002; 346:476-483, which is hereby incorporated by reference in its entirety). In addition, increased Hcy levels (>15 μmol/L) were shown to be present in up to 90% of patients with depression (see Bottiglieri et al. J Neurol Neurosurg Psychiatry 2000; 69:228-232; Bjelland et al. Arch Gen Psychiatry 2003; 60:618-626, both of which are hereby incorporated by reference in their entireties), with men in the upper tercile for tHcy levels being more than twice as likely to suffer depression as those in the lowest tercile (see Tolmunen et al. Am J Clin Nutr 2004; 80:1574-1578, which is hereby incorporated by reference in its entirety). An elevated Hcy level is commonly reported in cases of schizophrenia, multiple sclerosis, Parkinson's disease, fibromyalgia/chronic fatigue syndrome (see Kaeser et al. J Neurol Neurosurg Psychiatry 1969; 32:88-93; Golimbet et al. Psychiatry Res 2009; 170:168-171; Clarke et al. Arch Neurol 1998; 55:1449-1455; each of which is hereby incorporated by reference in its entirety) and recurrent dystonia without cerebrovascular disease (see Sinclair et al. Mov Disord 2006; 21:1780-1782, which is hereby incorporated by reference in its entirety). A possible association between T833C polymorphism of the CBS gene and bipolar disorder was described (see Permoda-Osip et al. Neuropsychobiology 2014; 69:107-111, which is hereby incorporated by reference in its entirety) and a recent meta-analysis indicated a relationship between elevated Hcy levels and mania/euthymia in individuals with bipolar disease (see Salagre et al. Eur Psychiatry 2017; 43:81-91, which is hereby incorporated by reference in its entirety). The first known case of peripheral neuropathy associated with HCU was recently described in an 18-year old man with HCU (see Oliveira et al. BMJ Case Rep 2016; 2016, which is hereby incorporated by reference in its entirety), and a lethal case of psychosis was described in a 17-year old with previously undiagnosed HCU (see Hidalgo et al. Eur Child Adolesc Psychiatry 2014; 23:235-238, which is hereby incorporated by reference in its entirety).

Direct evidence for a relationship between Hcy levels and dementia derive from animal studies in which Hcy administration was associated with development of brain lesions (see Troen et al. Proc Natl Acad Sci USA 2008; 105:12474-12479; Sudduth et al. J Cereb Blood Flow Metab 2013; 33:708-715, each of which is hereby incorporated by reference in its entirety). In the first such study, male C57BL6/J mice with elevated Hcy levels (induced by a vitamin B deficient diet) had significantly impaired spatial learning and memory, with significant rarefaction of the hippocampal microvasculature without concomitant gliosis or neurodegeneration (see Troen et al. Proc Natl Acad Sci USA 2008; 105:12474-12479, which is hereby incorporated by reference in its entirety). Total hippocampal capillary length was inversely correlated with Morris water maze escape latencies (r=−0.757, p<0.001) and with plasma tHcy (r=−0.631, p<0.007). Mice fed a Met rich diet showed similar, but less pronounced, effects. These findings suggested that elevated Hcy levels are associated with cerebral microvascular rarefaction leading to cognitive dysfunction in the absence of or preceding neurodegeneration. This may explain the link between elevated Hcy levels and cognitive decline in humans.

In the second study, healthy mice were fed a diet deficient in folate, vitamins B₆ and B₁₂ and supplemented with Met to induce moderately elevated Hcy levels (plasma tHcy 82.93±3.56 μmol/L). These mice had spatial memory deficits, as assessed with the two-day radial-arm water maze (see Sudduth et al. J Cereb Blood Flow Metab 2013; 33:708-715, which is hereby incorporated by reference in its entirety). MRI and histology revealed significant microhemorrhage rates. Neuroinflammation and increased expression and activity of MMP2 and MMP9, both enzymes implicated in pathogenesis of cerebral hemorrhage, were also observed. This suggested a link between elevated Hcy levels and vascular dementia, such as in Alzheimer's disease.

In humans, changes in white matter (a sign of vascular damage) are frequently associated with elevated Hcy levels, both in individuals with (see El Bashir et al. JIMD Rep 2015; 21:89-95; Vatanavicharn et al. J Inherit Metab Dis 2008; 31 Suppl 3:477-481; Brenton et al. J Child Neurol 2014; 29:88-92; Ruhoy et al. Pediatr Neurol 2014; 50:108-111; each of which is hereby incorporated by reference in its entirety) and without (see Hogervorst et al. Arch Neurol 2002; 59:787-793; Kloppenborg et al. Neurology 2014; 82:777-783; each of which is hereby incorporated by reference in its entirety) HCU. However, these changes are not always associated with evidence of stroke. In addition, brain imaging studies in HCU patients often show signs of atrophy or venous occlusion (see Vatanavicharn et al. J Inherit Metab Dis 2008; 31 Suppl 3:477-481, which is hereby incorporated by reference in its entirety).

The scan reveals diffuse symmetrical abnormal increased signal of the subcortical white matter and, to a lesser extent, of the deeper white matter, in the cerebral hemispheres, primarily the parieto-occipital regions

1. Mechanism

The precise mechanisms by which elevated Hcy levels affect neurological health are unknown. Several animal studies showed associations between elevated tHcy levels and neurotoxicity and accompanying nerve and mental impairment.

An early study showed that very high intraperitoneal doses of Hcy induced generalized convulsive status epilepticus in rats with cobalt-induced lesions in the motor cortex (see Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327, which is hereby incorporated by reference in its entirety). Seizures were enhanced by the addition of Met and vitamin B and there was some evidence for synergistic effects of pyridoxal 5′-phosphate and Hcy in blocking the postsynaptic g-aminobutyric acid receptor. Moreover, Hcy-treatment of rodent neocortical tissues led to adenosine trapping in the form of AdoHcy (see Heinecke et al. J Biol Chem 1987; 262:10098-10103, which is hereby incorporated by reference in its entirety). The authors stated that adenosine is predominantly a depressant in cerebral actions and that the convulsive conditions and that the mental changes associated with high levels of Hcy may be mediated by decreased levels of cerebral adenosine.

Hcy is associated with both neurotoxicity and morphological changes in the brain (see Hainsworth et al. Biochim Biophys Acta 2016; 1862:1008-1017, which is hereby incorporated by reference in its entirety). For example, studies in rats and rabbits showed that neuronal damage was caused by Hcy-mediated increases in thiobutyric acid reactive substances (TBARS), indicators of oxidative stress (see Stefanello et al. Metab Brain Dis 2007; 22:172-182; Toborek et al. Atherosclerosis 1995; 115:217-224, both of which are hereby incorporated by reference in its entirety). Similar increases in plasma TBARS were also observed in humans following an orally administered Met load (see Domagala et al. Thromb Res 1997; 87:411-416, which is hereby incorporated by reference in its entirety). Moreover, a study in a murine model for elevated Hcy levels suggested that cellular damage caused by oxidative stress may be enhanced in patients with HCU because decreased. Cys levels result in low levels of neuronal glutathione, an important antioxidant synthesized from glutamate, Cys and glycine (see Vivitsky et al. Am J Physiol Regul IntegrComp Physiol 2004; 287: R39-R46, which is hereby incorporated by reference in its entirety).

In vivo Hcy injection into the left ventricle of rat brains produced dose-dependent neuronal loss (see Yeganeh et al. J Mol Neurosci 2013; 50:551-557, which is hereby incorporated by reference in its entirety) and incubation of mesencephalic tegmental neurons from rats with Hcy led to fewer and shorter dopaminergic neurites (see Heider et al. J Neural Transm Suppl 2004; 1-13, which is hereby incorporated by reference in its entirety). In both studies, the effects of Hcy were blunted by co-administration of Hcy with antagonists of NMDA and metabotropic glutamate receptors, suggesting a glutamate-receptor mediated pathway for Hcy-induced neuronal damage. Further evidence for this pathway derives from a study in which Hcy administration led to dose-dependent lipid peroxidation in rat brain synaptosomes (see Jara-Prado et al. Neurotox Res 2003; 5:237-243, which is hereby incorporated by reference in its entirety). Once again, effects were inhibited by administration of an NMDA receptor antagonist.

A study in spontaneously active embryonic rat cortical neurons showed that Hcy levels above the range for severely elevated Hcy caused a dose-dependent suppression of neural network activity (see Gortz et al. J Neurol Sci 2004; 218:109-114, which is hereby incorporated by reference in its entirety). The effects observed in this study were not clinically relevant because such exaggerated Hcy levels are never reached in patients with HCU. However, modest elevations in homocysteinesulfinic acid and homocysteic acid (oxidized forms of Hcy often found in patients with elevated Hcy levels) had a similar effect. In each case, damage to the neural network was inhibited by 2-amino-5-phosphonovaleric acid, again implicating the NMDA receptor as a mediator of this Hcy induced neuronal dysfunction. These results suggested that the neuronal dysfunction associated with elevated Hcy levels is likely caused by oxidized forms of Hcy, rather than by Hcy itself.

The absence of cognitive symptoms, convulsions, and other CNS disorders in patients with Marfan syndrome and other connective tissue disorders suggest that the neurological disorders in HCU patients are not caused by defects in either fibrillin or collagen (see Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327, which is hereby incorporated by reference in its entirety). In patients with untreated HCU, elevated levels of SAM and decreased levels of SAH inhibit the transmethylation reactions required for myelin synthesis, leading to further nerve damage (see Mudd et al. Disorders of transsulfuration. In: Scriver C L, Beaudet A L, Sly W S, Valle D, eds. The Metabolic and Molecular Basis of inherited Diseases. 7 ed. New York: McGraw Hill; 2001; 1279-1327, which is hereby incorporated by reference in its entirety). Decreased myelin synthesis may be also be caused by low levels of serine in patients with HCU, due to increased remethylation rates (see Orendac et al. J Inherit Metab Dis 2003; 26:761-773, which is hereby incorporated by reference in its entirety). Finally, Hcy metabolism plays a key role in synthesis of monoamines by providing methyl groups for production of norepinephrine and dopamine (see Mech et al. J Clin Psychiatry 2016; 77:668-671; Folstein et al. Am J Psychiatry 2007; 164:861-867, both of which are hereby incorporated by reference in its entirety). Indeed, the “homocysteine hypothesis of depression” (see Folstein et al. Am J Psychiatry 2007; 164:861-867, which is hereby incorporated by reference in its entirety) states that low levels of norepinephrine and dopamine, resulting from elevated Hcy levels, are a major cause of depression.

Electron microscopy of rat brain biopsies showed cerebrovascular structural alterations in animals fed a high Hcy diet for 8 weeks. These alterations were associated with high plasma tHcy levels (see Lee et al. J Nutr 2005; 135:544-548, which is hereby incorporated by reference in its entirety). Consumption of dietary folic acid for a further 8 weeks decreased plasma tHcy to normal levels and significantly decreased the incidence of damaged vessels. This suggested that Hcy lowering, using folic acid supplementation, might reduce the detrimental effects on the vascular endothelium of experimentally-induced elevated Hcy levels.

A study designed to evaluate the efficacy and safety of reduced B vitamins as a monotherapy in adults with major depressive disorder (MDD) and HCU, due to at least one MTHFR polymorphism (N=330), found that treatment with a combination of reduced B vitamins significantly decreased tHcy levels in 131 treated patients (82.4%) (mean reduction in this subgroup was 25%; p <0.001), whereas placebo-treated patients demonstrated a small elevation in tHcy levels (see Mech et al. J Clin Psychiatry 2016; 77:668-671, which is hereby incorporated by reference in its entirety). Treated patients, on average, had a 12-point reduction on the Montgomery Asberg-Depression Rating Scale (MADRS) by Week 8, and 42% achieved full remission (p<0.001). Further clinical improvement was correlated with a significant decrease in tHcy levels in the majority of responders. Although this study was not conducted in patients with HCU, it demonstrated a clear benefit for Hcy lowering in individuals with depression, supporting the ‘Hcy hypothesis of depression’ (see Folstein et al. Am J Psychiatry 2007; 164:861-867, which is hereby incorporated by reference in its entirety).

The benefits of Hcy-lowering therapy in individuals with psychiatric symptoms associated with HCU were first demonstrated in a study of 12 late-diagnosed patients (see Grobe H. Eur J Pediatr 1980; 135:199-203, which is hereby incorporated by reference in its entirety). Three of these patients were never treated effectively and had serious psychological disorders, with premature death. The remaining 8 patients ranged from 1 to 26 years of age (mean 13 years) and all had psychiatric symptoms, including irritability, ADHD, apathy and psychosis. Treatment with pyridoxine or with a low Met diet with supplemental L-cystine for 2 to 9 years was associated with a striking improvement in behavior and intellectual development, correlated with biochemical normalization. The authors emphasized the need to treat all patients, regardless of age at diagnosis and prior treatment, because of the reversibility and improvement of the HCU associated sequelae observed in their study.

More recent data from the Irish screening program was used to compare the mental capabilities of 23 pyridoxine non-responsive individuals with HCU (339 patient-years of treatment) with those of 10 unaffected sibling controls (see Yap et al. J Inherit Metab Dis 2001; 24:437-447, which is hereby incorporated by reference in its entirety). Of the 23 patients identified, 19 were diagnosed with HCU through NBS and treated early in life (within 6 weeks of birth), two were late-detected (age 2.2 and 2.9 years) and two had been untreated at the time of assessment. All patients were treated with a Met-free, cysteine-supplemented synthetic amino acid mixture, with vitamin B₁₂ and folate supplements, as required. Betaine was used in the last 5 years as an adjunct to treatment in early-treated patients who became poorly compliant with diet and in all patients with late-detected HCU.

Overall, 13 of the 19 patients in the early-treated group (mean age 14.4 years; range 4.4 to 24.9) were compliant with treatment (defined by a lifetime plasma fHcy median <11 μmol/L) and had no complications, whereas the remaining 6 (mean age 19.9 years; range 13.8 to 25.5), who had poor compliance, developed complications. Mean full-scale IQ (FIQ) was 105.8 (range 84 to 120) in the compliant group compared with 80.8 (range 40 to 103) in the poorly compliant group. The control group (n=10) with a mean age of 19.4 years (range 9.7 to 32.9) years had a mean FIQ of 102 (range 76 to 116). The two late-detected patients, aged 18.9 and 18.8 years, had FIQ of 80 and 102, respectively, while the two untreated patients, aged 22.4 and 11.7 years, had FIQ of 52 and 53, respectively. There were no significant differences between compliant, early-treated individuals and their unaffected siblings (controls) except in FIQ, which was significantly higher in the affected siblings (p=0.0397). Despite the relatively small numbers, these results suggest that early treatment with good biochemical control prevents cognitive symptoms.

Similar results were obtained in a case-control study reporting on neurodevelopmental, educational and cognitive outcomes in 32 cases of HCU and 25 sibling controls in Qatar (see El Bashir et al. JIMD Rep 2015; 21:89-95, which is hereby incorporated by reference in its entirety). The mean age of subjects in this study was 11.2 years (range 0.6 to 29) and 56% were male. Compared with unaffected siblings, affected individuals had lower total IQ (particularly in terms of short-term memory, quantitative reasoning and visual—spatial domains) and a significant number of adolescents and adult cases had medical co-morbidities as well as behavioral and emotional problems. Of these, 9 cases (28%) of HCU were diagnosed by NB S and treated in the first month of life. The rest were diagnosed between 14 and 240 months of age. On-treatment tHcy and Met levels were significantly better in those diagnosed through NBS than in those diagnosed clinically, possibly because of better compliance with diets and medications early in life. A significant difference in IQ was observed between early- and clinically-diagnosed patients. Although the differences in language domains, attendance at special schools and access to extra support in class were not statistically significant between groups, the ‘clinically detected’ group clearly had more reported difficulties.

Although the numbers of patients studied here are small, a notable difference is seen between the children diagnosed at birth and those diagnosed as toddlers. An average tHcy level of 115 μmol/L, in the clinically diagnosed group was associated with poor clinical outcomes and very low IQs.

Further evidence for the benefits of Hcy-lowering treatment on psychopathology in HCU patients derives from a retrospective chart review of data from all patients with HCU presenting at Boston Children's Hospital since 1963 (unpublished data courtesy of M. Almuqbil, et al.). Overall, 19 patients with HCU were identified, three of which were excluded from the analysis because of the possibly confounding presence of methylmalonic acidemia (also associated with psychological defects) in addition to HCU. Of the remaining 16 patients, 7 (6 with HCU and one with cobalamin (Cbl) deficiency) had good compliance with early treatment (four on diet alone, two with diet plus betaine and one with Cbl). Six of these patients had no obvious psychiatric symptoms other than mild cognitive deficits. In contrast, 9 patients (7 with pyridoxine non-responsive HCU and two with CblG deficiency) had poor or variable compliance with treatment (two on betaine and diet, one on diet alone, three on B vitamins and two with folic acid and betaine). All 7 patients with HCU and poor compliance had psychiatric problems, including depression (n=4), paranoid experiences (n=2), paranoia and delusional psychosis (n=1), anxiety and mood dysregulation (n=1) and ADHD that improved with good metabolic control (n=1). The two CblG cases were markedly anxious or agitated. Age, gender and cognitive levels did not appear to significantly differentiate between the psychiatrically affected and non-affected individuals. These results suggest that good metabolic control (Hcy and/or Met lowering) has the potential to delay, and possibly prevent, the onset of psychiatric and behavioral conditions in HCU patients. However, the study did not ascertain whether poorly controlled HCU leads to psychopathology, or whether comorbidity with psychopathology itself hinders good compliance with treatment outcome.

Associations between elevated levels of Hcy and CNS symptoms, including cognitive symptoms, neurodegenerative diseases, seizures, dystonia, psychosis, cognitive impairment, dementia and depression, are well documented in individuals with and without HCU (see Morris et al. J Inherit Metab Dis 2017; 40:49-74; Abbott et al. Am J Med Genet 1987; 26:959-969; Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327; Hidalgo et al. Eur Child Adolesc Psychiatry 2014; 23:235-238; Schimke et al. JAMA 1965; 193:711-719; Smith et al. PLoS One 2010; 5: e12244; Seshadri et al. N Engl J Med 2002; 346:476-483; Bottiglieri et al. J Neurol Neurosurg Psychiatry 2000; 69:228-232; Bjelland et al. Arch Gen Psychiatry 2003; 60:618-626; Tolmunen et al. Am J Clin Nutr 2004; 80:1574-1578; Kaeser et al. J Neurol Neurosurg Psychiatry 1969; 32:88-93; Golimbet et al. Psychiatry Res 2009; 170:168-171; Clarke et al. Arch Neurol 1998; 55:1449-1455; Sinclair et al. Mov Disord 2006; 21:1780-1782; Permoda-Osip et al. Neuropsychobiology 2014; 69:107-111; Oliveira et al. BMJ Case Rep 2016; 2016; Troen et al. Proc Natl Acad Sci USA 2008; 105:12474-12479; Sudduth et al. J Cereb Blood Flow Metab 2013; 33:708-715; Hainsworth et al. Biochim Biophys Acta 2016; 1862:1008-1017; Herrmann et al. Clin Chem Lab Med 2011; 49:435-441; Kim et al. J Nutr 2007; 137:2093-2097; Selhub et al. Am J Clin Nutr 2000; 71:614S-620S; McCaddon et al. Dement Geriatr Cogn Disord 2001; 12:309-313; Smallwood et al. Neuropathol Appl Neurobiol 2012; 38:337-343; Beydoun et al. BMC Public Health 2014; 14:643; Gortz et al. J Neurol Sci 2004; 218:109-114; Health Quality O. Vitamin B12 and cognitive function: an evidence-based analysis. Ont.Health Technol.Assess.Ser.13 (23), 1e45. 2013. Ref Type: Online Source; Salagre et al. Eur Psychiatry 2017; 43:81-91, each of which is hereby incorporated by reference in its entirety). The mechanisms leading to CNS disorders in individuals with elevated levels of Hcy are believed to involve tHcy-mediated neuronal damage (see Mudd et al. Disorders of transsulfuration. In: Scriver C L, Beaudet A L, Sly W S, Valle D, eds. The Metabolic and Molecular Basis of Inherited Diseases. 7 ed. New York: McGraw Hill; 2001; 1279-1327; Hainsworth et al. Biochim Biophys Acta 2016; 1862:1008-1017; Stefanello et al. Metab Brain Dis 2007; 22:172-182; Toborek et al. Atherosclerosis 1995; 115:217-224, each of which is hereby incorporated by reference in its entirety), damage to the vascular endothelium caused by Hcy-mediated oxidative stress (see Vivitsky et al. Am J Physiol Regal IntegrComp Physiol 2004; 287: R39-R46, which is hereby incorporated by reference in its entirety), neuron loss (see Yeganeh et al. J Mol Neurosci 2013; 50:551-557; Heider et al. J Neural Transm Suppl 2004; 1-13, both of which are hereby incorporated by reference in its entirety) and attenuated neural network activity (see Gortz et al. J Neurol Sci 2004; 218:109-114, which is hereby incorporated by reference in its entirety). Depression and convulsions are thought to be caused, at least in part, by Hcy-mediated decreases in cerebral adenosine levels, with subsequent decreases in levels of norepinephrine and dopamine (see Mech et al. J Clin Psychiatry 2016; 77:668-671; Domagala et al. Thromb Res 1997; 87:411-416; Vivitsky et al. Am J Physiol Regul IntegrComp Physiol 2004; 287: R39-R46; Folstein et al. Am J Psychiatry 2007; 164:861-867; each of which is hereby incorporated by reference in its entirety).

Numerous studies, both in animal models for HCU (see Lee et al. J Nutr 2005; 135:544-548, which is hereby incorporated by reference in its entirety) and in patients with HCU (see El Bashir et al. JIMD Rep 2015; 21:89-95; Yap et al. J Inherit Metab Dis 2001; 24:437-447; Mech et al. J Clin Psychiatry 2016; 77:668-671; Grobe H. Eur J Pediatr 1980; 135:199-203, each of which is hereby incorporated by reference in its entirety), have shown that early decreases in Hcy levels, induced by a low Met diet, folic acid/B vitamin supplementation and/or pyridoxine/betaine therapy can prevent, and sometimes reverse, progression of various neurological disorders. Further evidence is provided by a series of 6 case studies in patients with HCU, in which significant decreases, even normalization, of Hcy levels resulted in complete or partial correction of CNS outcomes (see Yap et al. J Inherit Metab Dis 2001; 24:437-447; Brenton et al. J Child Neurol 2014; 29:88-92; Rezazadeh et al. Child Neurol Open 2014; 1:2329048X14545870; Kaeser et al. J Neurol Neurosurg Psychiatry 1969; 32:88-93; Colafrancesco et al. Eur J Pediatr 2015; 174:1263-1266; Yokoi et al. Pediatr Int 2008; 50:694-695; Li et al. Pathology 1999; 31:221-224, each of which is hereby incorporated by reference in its entirety).

These findings demonstrate a strong correlation between Hcy levels and an increased risk of CNS disorders in HCU patients and in the general population. Early Hcy-lowering therapy is essential for the normal development of children with early-onset HCU and for the correction or improvement of CNS disorders in patients diagnosed with HCU later in life.

### IX. Definitions

As used in this specification, the singular forms “a,” “an,” and “the” include plural references unless the context clearly dictates otherwise.

Where a range of values is provided, it is intended that each intervening value between the upper and lower limit of that range and any other stated or intervening value in that stated range is encompassed within the disclosure and specifically disclosed. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

As used herein, “co-administered” or “co-administration” means the administration of two or more therapeutic components, including a pharmaceutical composition.

As used herein, a “drug product” refers to a dosage form of a pharmaceutical composition including the drug substance of a PEGylated human truncated CBS protein with an amino acid sequence of SEQ ID NO: 1 (e.g., 20NHS PEG-CBS).

As used herein, a “drug substance” refers to a PEGylated CBS protein with an amino acid sequence of SEQ ID NO: 1 (e.g., 20NHS PEG-CBS).

As used herein, a “negative clinical outcome” refers to an undesirable phenotypic outcome resulting from a disease, disorder, or condition.

As used herein, “recombinant,” when used with reference to, e.g., a cell, nucleic acid, polypeptide, expression cassette or vector, refers to a material, or a material corresponding to the natural or native form of the material, that has been modified by the introduction of a new moiety or alteration of an existing moiety, or is identical thereto but produced or derived from synthetic materials. For example, recombinant cells express genes that are not found within the native (non-recombinant) form of the cell (i.e., “exogenous nucleic acids”) or express native genes that are otherwise expressed at a different level, typically, under-expressed or not expressed at all.

Recombinant techniques can include, e.g., use of a recombinant nucleic acid such as a cDNA encoding a protein or an antisense sequence, for insertion into an expression system, such as an expression vector; the resultant construct is introduced into a cell, and the cell expresses the nucleic acid, and the protein, if appropriate. Recombinant techniques also encompass the ligation of nucleic acids to coding or promoter sequences from different sources into one expression cassette or vector for expression of a fusion protein, constitutive expression of a protein, or inducible expression of a protein.

As used herein, the terms “subject”, “individual” or “patient” are used interchangeably and refer to a vertebrate, preferably a mammal. Mammals include, but are not limited to, humans.

As used herein, the term “non-genetically-defined,” as used in reference to a subject, a patient, and or a population of patients, refers to one or more subjects not having or not diagnosed as having a genetically-defined deficiency in cystathionine β-synthase (e.g., not having a missense or loss-of-function mutation in one or more CBS gene allele). For example, non-genetically defined subjects having elevated tHcy levels are subjects having tHcy levels above the normal range expected based on age, sex, dietary, and other factors, but not having or not being diagnosed as having a genetic deficiency in one or more CBS gene allele; i.e., not having genetically-defined HCU.

As used herein, “associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

As used herein, “pharmaceutically acceptable excipient” or “pharmaceutically acceptable carrier” refers to an excipient that may optionally be included in the compositions of the disclosure and that causes no significant adverse toxicological effects to the patient. In particular, in the present instance, such refers to an excipient that can be taken into the mammalian subject's body in association with an active compound (here PEGylated htCBS or “20NHS PEG-CBS”) with no significant adverse toxicological effects to the subject.

As used herein, the terms “adjuvant,” “diluent,” or “carrier” mean any substance, not itself a therapeutic agent, used as a carrier for delivery of a therapeutic agent and suitable for administration to a subject, e.g. a mammal or added to a pharmaceutical composition to improve its handling or storage properties or to permit or facilitate formation of a dose unit of the composition into a discrete article such as a capsule or tablet suitable for oral administration. The terms “adjuvant,” “diluent,” or “carrier” encompass “excipients,” including “pharmaceutically acceptable excipients,” “vehicles,” “solvents,” and the like, as those terms are used herein. Excipients and vehicles include any such materials known in the art, e.g., any liquid, gel, solvent, liquid diluent, solubilizer, or the like, which is non-toxic, and which does not interact with other components of the composition in a deleterious manner. Administration can mean oral administration, inhalation, enteral administration, feeding or inoculation by intravenous injection. The excipients may include standard pharmaceutical excipients and may also include any components that may be used to prepare foods and beverages for human and/or animal consumption, feed or bait formulations or other foodstuffs.

As used herein, “drug” or “active agent” or any other similar term means any chemical or biological material or compound, inclusive of peptides, suitable for administration by the methods previously known in the art and/or by the methods taught in the present disclosure, that induces a desired biological or pharmacological effect, which may include, but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anesthetic effect, or it may be systemic.

As used herein, the term “about,” particularly in reference to a given quantity, is meant to encompass deviations of plus or minus five percent.

As used herein, the terms “therapeutically effective amount” as related to the present composition refer to a non-toxic, but sufficient amount of the active agent (or composition containing the active agent) to provide the desired level in the bloodstream or at the site of action (e.g. intracellularly) in the subject to be treated, and/or to provide a desired physiological, biophysical, biochemical, pharmacological or therapeutic response, such as amelioration of the manifestations of homocystinuria. The exact amount required will vary from subject to subject, and will depend on numerous factors, such as the active agent, the activity of the composition, the delivery device employed, the physical characteristics of the composition, intended patient use (i.e., the number of doses administered per day), as well as patient considerations, such as species, age, and general condition of the subject, the severity of the condition being treated, additional drugs being taken by the subject, mode of administration, and the like. These factors and considerations can readily be determined by one skilled in the art, based upon the information provided herein. An appropriate “effective” amount in any individual case may be determined by one of ordinary skill in the art using routine experimentation, based upon the information provided herein.

As used herein, the term “nucleic acid” may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

As used herein, a “mutant” is a mutated protein designed or engineered to alter properties or functions relating to glycosylation, protein stabilization and/or ligand binding.

As used herein, the terms “native” or “wild type” relative to a given cell, polypeptide, nucleic acid, trait or phenotype, refers to the form in which that is typically found in nature.

As used herein, the terms “protein,” “polypeptide,” “oligopeptide” and “peptide” have their conventional meaning and are used interchangeably to denote a polymer of at least two amino acids covalently linked by an amide bond, regardless of length or post-translational modification (e.g., glycosylation, phosphorylation, lipidation, myristylation, ubiquitination, etc.). Furthermore, the polypeptides described herein are not limited to a specific length. Included within this definition are D- and L-amino acids, and mixtures of D- and L-amino acids. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Polypeptides can also refer to amino acid subsequences comprising epitopes, i.e., antigenic determinants substantially responsible for the immunogenic properties of a polypeptide and being capable of evoking an immune response.

As used herein, “position corresponding to” and the like refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 90%, greater than 95%, greater than 96%, greater than 97%, greater than 98% or greater than 99%. The position of interest is then given the number assigned in the reference nucleic acid molecule. For example, if a particular polymorphism in Gene-X occurs at nucleotide 2073 of SEQ ID NO: X, to identify the corresponding nucleotide in another allele or isolate, the sequences are aligned and then the position that lines up with 2073 is identified. Since various alleles may be of different length, the position designating 2073 may not be nucleotide 2073, but instead is at a position that “corresponds” to the position in the reference sequence.

As used herein, the term “long-term administration” refers to administration of the CBS enzyme, htCBS, or htCBS mutant (e.g., with a C15S mutation) conjugated to a PEG moiety over a time-period of 6 weeks or longer. The term “long-term continuous treatment” refers to repeated administration of the CBS enzyme, htCBS, or htCBS mutant (e.g., with a C15S mutation) conjugated to a PEG moiety throughout the course of a study via subcutaneous injection or implanted osmotic pump.

Described herein are methods of treating homocystinuria and/or CBS deficiency through enzyme therapy (ET) with the drug product described herein including a PEGylated human truncated CBS protein with a mutation at amino acid position 15 of a cysteine to a serine.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Natural History Study Overview and Patient Characteristics

CBS-HCY-NHS-01 is an ongoing, multicenter (8 sites), international, observational, prospective natural history study (“NETS”) of HCU that enrolled 55 pediatric (5-17 years of age) and adult (>18 years of age) patients to characterize the clinical course of HCU in patients under current clinical management practices over 3 years to understand how homocystinuria progresses over time and to identify new treatments for patients living with homocystinuria. CBS-HCY-NHS-01 explores the range of plasma concentrations of total Hcy (tHcy) and related sulfur metabolites, and the variability of the clinical sequelae of the disease. Interim analyses assessing patient characteristics, cognitive impairments, and skeletal abnormalities were performed. Observed patient characteristics are provided in Table 1.

TABLE 1
Patient demographics and baseline characteristics

| Patient characteristic | Prevalence in all patients | |
|---|---|---|
| Age at enrollment, median [range] | 21.0 | [5-53] |
| Pediatric (<18 years), N (%) | 23 | (42%) |
| Adult (≥18 years), N (%) | 32 | (58%) |
| Sex: Male/Female, % | 55%/45% | |
| Race/Ethnicity, N (%) | | |
| White | 51 | (93%) |
| African-American | 3 | (6%) |
| Hispanic or Latino | 6 | (11%) |
| Non-Hispanic | 46 | (84%) |
| Adult weight, median [range], kg | 80.5 | [55-138] |
| Adult height, median [range], cm | 176 | [148-200] |
| BMI, median [range], kg/m² | 21.5 | [15.0-49.5] |
| Diagnosed at <1 year of age, N (%) | | |
| Yes | 28 | (51%) |
| No | 25 | (46%) |
| Have family history of HCU, N (%) | | |
| Yes | 20 | (36%) |
| No | 35 | (64%) |
| Adult patients' highest education level | | |
| Some high school | 14% | |
| High school graduate | 21% | |
| Some college/college graduate | 41% | |
| Master's degree | 14% | |
| Professional degree | 3% | |
| Unknown | 7% | |
| tHcy at diagnosis,^{†} median [range], μM | | |
| Adults (≥18 years) | 100 | [5-364] |
| Pediatric (<18 years) | 144 | [35-298] |
| Natural protein-restricted diet, N (%) | 38 | (93%) |
| Adults (≥18 years) | 20 | (91%) |
| Pediatric (<18 years) | 18 | (95%) |
| Met-free L-amino acid mixture, N (%) | 32 | (58%) |
| Adults (≥18 years) | 15 | (47%) |
| Pediatric (<18 years) | 17 | (74%) |
| B-vitamin supplements, N (%) | 40 | (83%) |
| Adults (≥18 years) | 19 | (76%) |
| Pediatric (<18 years) | 21 | (91%) |
| Betaine supplement, N (%) | 41 | (85%) |
| Adults (≥18 years) | 21 | (84%) |
| Pediatric (<18 years) | 20 | (87%) |
| Time on study, median [range], months | 12.2 | [0.03^{$}-24.8] |

^{†}Historic tHcy data were available for 33 patients only, self-reported.
^{$}One subject has only undergone Visit 1.

Fifty-five patients with HCU were enrolled in this natural history study. The median time on study was 12.2 months at the time of the interim analysis. The disease affects both sexes similarly (55% males, 45% females). HCU patients in the study were young (median age: 21.0 years old); 42% were pediatric patients (<18 years of age) and 58% were adult patients. Median BMI of this population is in the normal range (21.5 kg/m2). Half of the patients (51%; 65% of pediatric; 41% of adult) were diagnosed in the first year of life (<1 year of age). 36% of patients had a family history of HCU. The median plasma tHcy level was 95 μM (74 μM for pediatric patients; 104 μM for adult patients), consistent with underlying undertreated disease. 95% of pediatric patients had tHcy greater than the upper limit of normal (ULN) and 82% had tHcy levels >50 μM (82%). The tHcy levels of almost half (45%) of the pediatric patients were >100 μM. All (100%) adult patients had tHcy levels greater than the ULN, 93% had tHcy levels >50 and 77% had tHcy >100 μM (77%). Based on 3-day diet diaries recorded prior to each clinic visit, most patients (93%; 95% pediatric; 91% adult) were following a natural protein-restricted diet and most were taking a Met-free L-amino acid mixture (58%; 74% pediatric; 47% adult). Most patients (83%; 91% of pediatric; 76% of adult) were taking B vitamins supplements. Most patients (85%; 87% of pediatric; 84% of adult) were taking betaine (Table 1).

### Example 2. NHS—Opthalmic Defects

Historically, patients have often been diagnosed due to ectopia lentis. In the largest retrospective survey conducted to date, 85% of HCU patients had developed this condition by the age of 20 (Mudd S H, et al. The natural history of homocystinuria due to cystathionine beta-synthase deficiency. Am J Hum Genet. 1985; 37(1):1-31). In contrast, only 19% of adult and 9% of pediatric patients in this managed population had this condition (see Table 2), suggesting that restricted diet, B vitamin and betaine supplement can be effective mitigators of ophthalmic defects despite underlying undertreated disease as evidenced by median plasma tHcy level.

TABLE 2
Ophthalmic deficits

| | | | Pediatric | | Adult | |
|---|---|---|---|---|---|---|
| | All Patients | | (5-17 years) | | (≥18 years) | |
| | N = 55 | | N = 23 | | N = 32 | |
| Any ocular deficit | 38 | (69%) | 16 | (70%) | 22 | (69%) |
| Ectopia lentis/ | 8 | (15%) | 2 | (9%) | 6 | (19%) |
| dislocation of the lens | | | | | | |
| Cataract | 6 | (11%) | 2 | (9%) | 4 | (13%) |
| Retinal degeneration | 1 | (2%) | 0 | (0%) | 1 | (3%) |
| Retinal pigmentosa | 1 | (2%) | 1 | (4%) | 0 | (0%) |
| Hyperopia | 11 | (28%) | 7 | (30%) | 4 | (13%) |
| Myopia | 25 | (63%) | 9 | (39%) | 16 | (50%) |

### Example 3. NHS—CBS Gene Mutations

Analysis of mutations in the CBS gene of study participants showed 26 unique mutations identified in 48 patients, with 16 patients apparently homozygotes and 32 patients apparently compound heterozygotes. The DNA mutations and resulting CBS mutant proteins are shown in Table 3 using standard nomenclature for genetic mutations as described by Ogino, Shuji, et al. “Standard mutation nomenclature in molecular diagnostics: practical and educational challenges.” The Journal of molecular diagnostics 9.1 (2007): 1-6, the disclosure of which is incorporated by reference herein in its entirety. Genetically-defined HCU patients include patients having a mutation in the CBS gene, including but not limited to any of the mutations shown in Table 3.

TABLE 3
Patient mutations in the CBS gene

| DNA | Protein |
|---|---|
| c.689del | p.Leu230Argfs*39 |
| c.209 + 1G > A | p.(?) |
| c.1126G > A | p.Asp376Asn |
| c.752T > A | p.Leu251Gln |
| c.808_810del | p.Glu270del |
| c.442G > A | p.Gly148Arg |
| c.536_553del | p.Asp179_Leu184del |
| c.700G > A | p.Asp234Asn |
| c.1106G > C | p.Arg369Pro |
| c.325T > C | p.Cys109Arg |
| c.361C > T | p.Arg121Cys |
| c.1006C > T | p.Arg336Cys |
| c.785C > T | p.Thr262Met |
| c.1058C > T | p.Thr353Met |
| c.1039G > A | p.Gly347Ser |
| c.770C > T | p.Thr257Met |
| c.153_165del | p.Arg51Serfs*27 |
| c.1224-2A > C | p.(?) |
| c.919G > A | p.Gly307Ser |
| c.1330G > A | p.Asp444Asn |
| c.833T > C | p.Ile278Thr |
| c.1152G > C | p.Lys384Asn |
| c.1339C > T | p.Pro447Ser |
| c.488A > G | p.Tyr163Cys |
| c.624G > A | p.Trp208* |
| c.738del | p.Lys274Serfs*2 |
| c.1136G > A | p.Arg379Gln |
| c.1397C > A | p.? |
| c.162G > A | p.Trp54Ter |
| c.19dupC | p.Gln7ProfsX30 |
| c.302T > C | p.Leu101Pro |
| c.362G > A | Arg121His |
| c.667-14_667- | p? |
| 7delCTCTTTCT | |
| c.685C > A | p.Pro229Thr |
| c.816T > A | pCys272ter |
| c.829-78_1146- | p.? |
| 273delins469 | |
| c.982G > A | p.? |

### Example 4. NHS—tHcy Levels

tHcy levels were determined at each visit (Table 4). Most patients (96%) had plasma tHcy levels 5 to 40 times the ULN of 14 μM for >12 years of age and the ULN of 9.6 μM for 0-12 years of age. Intra-subject variability was moderate. Overall, patients with low tHcy levels at Visit 1 tended to have low tHcy levels throughout the study (and vice-versa) (Pearson's R=0.6294). Large between-visit variabilities observed in some patients may be attributed to a change of diet/therapy compliance between visits, highlighting the challenge in maintaining patient compliance with restricted diets and dietary supplements.

TABLE 4
tHcy levels during study

| Patients | N | Median (μM) | Minimum (μM) | Maximum (μM) |
|---|---|---|---|---|
| All | 52 | 95 | 2 | 402 |
| Pediatric | 22 | 74 | 2 | 361 |
| (<18 years) | | | | |
| Adult | 30 | 104 | 10 | 402 |
| (≥18 years) | | | | |

### Example 5. NHS—Other Metabolic Indicators

Various laboratory values were determined during the study (see FIG. 1 and Table 5). High tHcy and Met, and low cystathionine and total cysteine levels, which are hallmarks of the disease, were observed despite most patients following standard of care (protein-restricted diet, B-vitamins, supplements, betaine). Methionine levels >1000 μM were observed in 11% of patients (14% pediatric, 10% adult) and ≥600 μM in 33% of patients (36% pediatric, 33% adult). High betaine, vitamin B12, and B6 levels were observed as expected as most patients take those supplements. Dimethylglycine (DMG) levels above the ULN were observed in 76% of patients, which could lead to a higher risk of acute myocardial infarction in some patients (Svingen et al. 2013). ALT levels above the ULN were observed in 37% of patients (52% pediatric; 28% adult). Creatinine levels below the lower limit of normal (LLN) were observed in 43% of patients (74% pediatric, 21% adult), and may be due to low muscle mass secondary to protein restriction. hsCRP levels above the ULN were observed in 35% of patients tested (N=40; only tested in patients ≥13 years of age, only observed in patients ≥18 years of age). Low Protein C activity levels were observed in 28% of patients, and low fibrinogen levels were observed in 31% of patients (N=29; both only tested in patients ≥13 years of age, only observed in patients ≥18 years of age).

Normal levels were observed for: AST (89% of patients) (all patients tested); anti-thrombin III (83% of patients), and apolipoprotein A (93% of patients) (only patients ≥13 years of age were tested for these laboratory parameters); bone-specific alkaline phosphatase (97% of patients), serum CTX (95% of patients), and P1NP (87% of patients) (only patients >18 years of age were tested for these laboratory parameters).

Additional laboratory values are provided in FIG. 1, wherein bars indicate the number of patients with a high/normal/low laboratory value.

TABLE 5
Selected abnormal laboratory values (all
laboratory ranges are age-adjusted)

| | | | Pediatric | | Adults | |
|---|---|---|---|---|---|---|
| | All patients | | (<18 years) | | (≥18 years) | |
| tHcy > ULN | 51 | (98%) | 21 | (95%) | 30 | (100%) |
| tHcy > 50 μM | 46 | (88%) | 18 | (82%) | 28 | (93%) |
| tHcy > 100 μM | 33 | (63%) | 10 | (45%) | 23 | (77%) |
| Methionine > ULN | 45 | (88%) | 20 | (91%) | 23 | (85%) |
| Methionine > 1,000 μM | 6 | (11%) | 3 | (14%) | 3 | (10%) |
| Methionine ≥ 600 μM | 18 | (33%) | 8 | (36%) | 10 | (33%) |
| Cystathionine < LLN | 48 | (94%) | 20 | (95%) | 26 | (93%) |
| DMG > ULN | 41 | (79%) | 19 | (86%) | 20 | (71%) |
| ALT > ULN | 20 | (37%) | 12 | (52%) | 8 | (28%) |
| Creatinine < LLN | 23 | (43%) | 17 | (74%) | 6 | (21%) |
| hsCRP* > ULN | 14 | (35%) | 0 | (0%)** | 13 | (45%)** |
| hsCRP* > 2x ULN | 10 | (25%) | 0 | (0%)** | 9 | (31%)** |
| Protein C activity* < LLN | 8 | (28%) | 0 | (0%) | 8 | (28%) |
| Fibrinogen* < LLN | 9 | (31%) | 0 | (0%) | 9 | (31%) |

*hsCRP, Protein C activity and fibrinogen were only tested in patients ≥ 13 years of age.
**Age was missing for one patient.

Table 5 and FIG. 1 demonstrate that in addition to tHcy, methionine, cystathionine, DMG, ALT, creatinine, hsCRP, Protein C activity, fibrinogen, ALT-SGPT, betaine, cystathionine, plasma vitamin B6, total cysteine, and vitamin B12, among others, may be useful as metabolic indicators of disease severity or progression, or otherwise indicate disease severity or progression in an individual. These patient characteristics demonstrate that, despite being seen and treated at centers of excellence and being prescribed a natural protein-restricted diet and/or a Met-free L-amino acid mixture and supplements, many patients have plasma tHcy values 5 to 40 times the ULN for tHcy, hypermethioninemia (≥6000 μM) (observed in 33% of patients: 36% pediatric, 33% adult), ocular deficits, signs of inflammation, protein metabolism and/or liver dysfunction. These data indicate that current diet and therapeutic interventions are poorly effective and/or that most patients are not able to remain compliant, leading to high tHcy levels, even in patients being frequently monitored at centers of excellence. In particular, these data also point out that betaine does not result in appropriate control of the tHcy levels in HCU and, therefore, is not in itself adequate treatment for HCU, contrary to common belief. Moreover, this study identified new CBS gene mutations (Table 3) and new laboratory markers or metabolic indicators of disease severity or disease progression (Table 5 and FIG. 1) that are expected to be useful in diagnosing HCU, monitoring disease progression, monitoring patient compliance and reaction to treatments, assessing quality of life, tailoring therapies to suit specific patient profiles, and in clinical trials to determine the efficacy of new treatments.

### Example 6. NHS—Skeletal Fragility

Bone mass density (BMD) was assessed at baseline and 1 year at three locations (the hip, lumbar spine, and whole body) by dual-energy X-ray absorptiometry (DXA) using either Hologic or General Electric/Lunar densitometers. BMD and Z-scores (number of standard deviations away from the average BMD value of the reference group, normalized for age and gender) were calculated for each location. A Z-score between −1 and −2.5 indicates osteopenia, while a Z-score below −2.5 indicates osteoporosis. tHcy levels were measured in plasma, and median total protein intake (g/day) was calculated from 3-day food records. Total protein intake was defined as the sum of the total natural protein from the diet and the protein from Met-free L-amino acid mixture, if taken by the patient. Correlations between BMD and tHcy or dietary protein, were calculated using the Pearson's correlation coefficient (R). Table 6 shows the percentile ranks associated with different Z-scores.

TABLE 6
Z-score and percentile rank

| Percentile | Z-score |
|---|---|
| 5 | −1.65 |
| 10 | −1.29 |
| 15 | −1.04 |
| 20 | −0.84 |
| 25 | −0.68 |
| 30 | −0.53 |
| 25 | −0.39 |
| 40 | −0.26 |
| 45 | −0.13 |
| 50 | 0 |
| 55 | 0.13 |
| 60 | 0.26 |
| 65 | 0.39 |
| 70 | 0.53 |
| 75 | 0.68 |
| 80 | 0.84 |
| 85 | 1.04 |
| 90 | 1.29 |
| 95 | 1.65 |

BMD data were available for 43 patients. The median Z-score was negative for both adult and pediatric patients at all body locations (Table 7), indicating skeletal fragility. 46% of adult and 53% of pediatric patients had bone densities below the 15^{th} percentile (Z-scores ≤−1) in at least one location (Table 7). BMD results and Z-scores were stable over 12 months for each patient (N=15-20; correlation between Visit 1 and Visit 3 for BMD at each location: Pearson's R=0.94 to 0.97; p<0.0001).

TABLE 7
Summary of Z-score results by DXA scan location

| | Hip | | | Spine | | | Body | | |
|---|---|---|---|---|---|---|---|---|---|
| | All | | | All | | | All | | |
| | patients | Adult | Pediatric | patients | Adult | Pediatric | patients | Adult | Pediatric |
| N | 43 | 24 | 19 | 42 | 23 | 19 | 37 | 19 | 18 |
| Mean | −0.76 | −0.60 | −0.97 | −0.52 | −0.63 | −0.39 | −0.72 | −0.81 | −0.62 |
| SD | 1.32 | 1.38 | 1.26 | 1.30 | 1.44 | 1.15 | 1.37 | 1.36 | 1.20 |
| Median | −0.9 | −0.75 | −1.00 | −0.65 | −0.80 | −0.50 | −0.8 | −1.00 | −0.65 |
| Range | −2.6 | −2.4 | −2.6 | −3.2 | −3.2 | −2.3 | −3.0 | −3.0 | −2.9 |
| | to 3.0 | to 3.0 | to 1.9 | to 3.2 | to 3.2 | to 1.9 | to 2.2 | to 2.2 | to 1.3 |
| N (%) | 21 | 11 | 10 | 14 | 10 | 4 | 17 | 10 | 7 |
| patients | (48.8%) | (45.8%) | (52.6%) | (33.3%) | (43.5%) | (21.1%) | (45.9%) | (52.6%) | (38.9%) |
| with Z- | | | | | | | | | |
| score | | | | | | | | | |
| ≤−1.0 | | | | | | | | | |

Patients with plasma tHcy levels <100 μM had much greater skeletal fragility than those with levels >100 μM (FIG. 2). There was a positive correlation between plasma tHcy levels and BMD at all three locations (Pearson's R=0.33 to 0.51; p<0.03; Table 8). These data demonstrate that BMD improves with greater total dietary protein intake, as shown by a positive correlation between BMD at all three locations and total dietary protein intake (Pearson's R=0.55 to 0.76; p<0.011; Table 8). There is a positive correlation between BMD at all three locations and C-reactive protein (hsCRP) (Pearson's R=0.36 to 0.39; p<0.051; Table 8), a marker of inflammation, suggesting that inflammation may be correlated with high tHcy or increased protein intake.

TABLE 8
Correlations between BMD and other parameters

| | | | | |
|---|---|---|---|---|
| | N (all, | | | |
| | adult, | All | | |
| BMD | pediatric) | Subjects | Adult | Pediatric |

Pearson Correlation with tHcy

| | | | | |
|---|---|---|---|---|
| Hip | 45, 26, 19 | 0.3340 | 0.1545 | 0.4963 |
| | | (p = 0.0249) | (p = 0.4510) | (p = 0.0307) |
| Spine | 41, 24, 17 | 0.4784 | 0.5001 | 0.5005 |
| | | (p = 0.0016) | (p = 0.0128) | (p = 0.0408) |
| Body | 38, 19, 19 | 0.5095 | 0.4772 | 0.3939 |
| | | (p = 0.0011) | (p = 0.0388) | (p = 0.0952) |

Pearson Correlation with hsCRP

| | | | | |
|---|---|---|---|---|
| Hip | 33, 26, 7 | 0.3597 | 0.3398 | 0.3334 |
| | | (p = 0.0398) | (p = 0.0894) | (p = 0.4649) |
| Spine | 30, 24, 6 | 0.3611 | 0.2875 | 0.6717 |
| | | (p = 0.0500) | (p = 0.1732) | (p = 0.1440) |
| Body | 26, 19, 7 | 0.3925 | 0.2999 | 0.2123 |
| | | (p = 0.0473) | (p = 0.2122) | (p = 0.6476) |

Pearson Correlation with Total Dietary Protein

| | | | | |
|---|---|---|---|---|
| Hip | 24, 11, 13 | 0.7566 | 0.7268 | 0.6843 |
| | | (p < 0.0001) | (p = 0.0113) | (p = 0.0099) |
| Spine | 22, 10, 12 | 0.6870 | 0.7073 | 0.5532 |
| | | (p = 0.0004) | (p = 0.0221) | (p = 0.0621) |

These data demonstrate that most HCU patients have skeletal fragility despite a relatively young age, confirming past studies (Parrot F, et al. J Inherit Metab Dis. Osteoporosis in late-diagnosed adult homocystinuric patients. 2000; 23:338-40; Weber D R, et al. Low bone mineral density is a common finding in patients with homocystinuria. Med Genet Metab. 2016; 117:351-4). For each patient, BMD results and Z-scores were stable over 12 months. Therefore, BMD and Z-scores assessed by DXA can be considered a reliable endpoint to assess the efficacy of investigational treatments in future clinical trials in HCU patients. Bone health in this population is correlated with higher total dietary protein intake and higher tHcy levels, suggesting that, by chronically restricting their total protein intake to control their tHcy levels, patients may be increasing their skeletal fragility.

In general, HCU patients have greater skeletal fragility than the healthy population. However, as discussed above, within patients, higher tHcy levels are positively correlated with bone mass. Accordingly, skeletal symptoms in subjects having elevated tHcy levels, including non-genetically defined CBS deficient subjects, can be treated using the methods described herein to alleviate skeletal symptoms associated with elevated tHcy levels.

### Example 7. NHS—Cognition

Previous research has demonstrated associations between lifetime total Hcy (tHcy) as a key predictor of intellectual functioning in HCU (Al-Dewik N, et al. Natural history, with clinical, biochemical and molecular characterization, of classical homocystinuria in the Qatari population. J Inherit Metab Dis. 2019; April 10; Yap S, et al. The intellectual abilities of early-treated individuals with pyridoxine-nonresponsive homocystinuria due to cystathionine β-synthase deficiency. J Inher Metabol Dis. 2001; 24(4):437-47). Missing from the current literature is a description of profiles of cognitive strengths or deficits as well as exploration of whether proximal biomarkers of HCU and cognitive ability are associated.

Executive functions refer to a set of top-down mental processes that are effortful and needed for attention and concentration (Diamond A. Executive functions. Annu Rev Psychol. 2013; 64:135-68). Executive functioning is particularly sensitive to changes in physical health and is a good candidate to evaluate associations with biomarkers of HCU severity. Unlike overall intelligence, executive functioning can be improved through intervention (Diamond 2013). Cognitive function was assessed at baseline and every 6 months using the age-normalized NIH Toolbox Cognition Battery (NIHCB), which assesses language, working memory, episodic memory, processing speed, set shifting, and inhibitory control (Weintraub S, et al. Cognition assessment using the NIH Toolbox. Neurol 2013:80(11):S54-64; Weintraub et al. NIH Toolbox Cognition Battery (CB): introduction and pediatric data. Monogr Soc Res Child Dev. 2013; 78(4):1-15). The analyses used the median scores from the visits. Patient data from 1 to 5 visits were available. tHcy levels were measured in plasma. Correlations between cognitive function and tHcy, Cys and Met were calculated using the Pearson's correlation coefficient (r).

Cognitive function data were available for 51 patients. The overall cognitive function of HCU patients was severely affected (median Total Cognition Composite at 20^{th} percentile; 21^{st} percentile for adult and 14^{th} percentile for pediatric patients). Areas of cognition that were impacted included Fluid Cognition Composite, Executive Function, Memory, and Processing Speed. The Fluid Cognition Composite is a summary score of memory, executive functioning, and processing speed. It includes the capacity for new learning and information processing. Median Fluid Cognition Composite was found to be in the 10^{th} percentile in the present study. Executive Function represents inhibition of automatic response tendencies and capacity to switch behavior based on task demands. It includes Set Shifting and Inhibitory Control. Median Executive Function in the present study was found to be in the 18^{th} percentile for Set Shifting and 9^{th} percentile for Inhibitory Control. Memory represents capacity to hold information in a short-term buffer and manipulate the information as well as ability to acquire, store, and retrieve information. It includes Working Memory and Episodic Memory. Median Memory in the present study was found to be at the 24^{th} percentile for Working Memory and 32^{nd} percentile for Episodic Memory. Processing Speed represents mental efficiency for taking in information. Median Processing Speed was found to be in the 24^{th} percentile in the present study.

Language processing function (Receptive Vocabulary and Word Reading) and areas of cognition that reflect past learning and knowledge (Crystallized Cognition Composite) were within normal range.

NIH Toolbox results were consistent from visit to visit (CV<10% for the majority of patients). Intra-class correlations (ICC) ranged from 0.73 (Inhibitory Control) to 0.89 (Total Cognition Composite), with the exception of Episodic Memory with an ICC of 0.64. The original validation of the NIH Toolbox considered ICCs of 0.4 to 0.74 to be adequate and above 0.75 to be excellent. FIG. 3 shows NIH Toolbox median and quartile scores for tested cognitive functions.

Correlations between Cognition and Other Parameters were determined. tHcy levels were negatively correlated with overall cognition (Total Cognition Composite) (r=−0.32; p=0.023; Table 9); i.e., the higher the tHcy levels, the lower the cognition score. Inhibitory Control was the domain most impacted by tHcy levels (r=−0.33; p=0.019; Table 9).

Although the overall cognitive function of HCU patients was generally severely affected (r=−0.32; p=0.023; Table 9), the impairment increased with increasing tHcy levels. Patients with tHcy levels >100 μM had overall much poorer cognition than those with tHcy ≤100 μM in all cognition areas. When comparing patients with tHcy ≤100 μM to patients with tHcy>100 μM the domains for Inhibitory Control and Receptive Vocabulary showed the most statistically significant differences (FIG. 4). In contrast, plasma Met levels were negatively correlated with overall cognition (r=−0.28; p=0.049; Table 9), suggesting that both tHcy and Met levels should be kept as close to normal as possible. Cys levels were positively correlated with overall cognition (r=0.37; p=0.008; Table 9).

TABLE 9
Correlation between cognition and other parameters

| | Pearson's | | | |
|---|---|---|---|---|
| Cognition areas | parameters | Plasma tHcy | Plasma Cys | Plasma Met |
| Set | r | −0.2787 | 0.1547 | −0.2001 |
| Shifting | p | 0.0476 | 0.2783 | 0.1591 |
| | n | 51 | 51 | 51 |
| Inhibitory | r | −0.3288 | 0.2223 | −0.1987 |
| Control | p | 0.0185 | 0.117 | 0.1622 |
| | n | 51 | 51 | 51 |
| Processing | r | −0.1526 | 0.0565 | 0.0620 |
| Speed Test | p | 0.2852 | 0.6939 | 0.6659 |
| | n | 51 | 51 | 51 |
| Episodic | r | −0.1372 | 0.3245 | −0.3374 |
| Memory | p | 0.337 | 0.0202 | 0.0155 |
| | n | 51 | 51 | 51 |
| Fluid | r | −0.28554 | 0.24182 | −0.23209 |
| Cognitive | p | 0.042 | 0.087 | 0.101 |
| Composite | n | 51 | 51 | 51 |
| Total | r | −0.3181 | 0.36741 | −0.27767 |
| Cognitive | p | 0.0229 | 0.008 | 0.0485 |
| Composite | n | 51 | 51 | 51 |

Patients with lower plasma tHcy levels performed better on measures of executive functioning, extending prior studies showing that controlling tHcy levels is essential for maintaining intellectual functioning within average range (Walter J H, et al. Strategies for the treatment of cystathionine β-synthase deficiency: the experience of the Willink Biochemical Genetics Unit over the past 30 years. Eur J Pediatr. 1998; 157(2):571-6; Yap et al. 2001, Al-Dewik et al. 2019).

This study demonstrates that the NIH Toolbox is a reliable instrument that has demonstrated potential value for assessing cognitive functioning over time in patients with HCU, including tracking response to intervention.

The results of this study have practical implications for the management of HCU. Neuropsychological evaluations are shown to be an important component of medical care for HCU patients, and assessment of executive functioning (including response inhibition) should be included as part of those evaluations. For children with HCU, supports similar to those for children with other executive functioning problems, such as ADHD, should be considered.

Identification of specific cognitive deficits in HCU provides clues for future studies focused on neural systems impacted by HCU. For example, dorsal anterior cingulate cortex activation is associated with monitoring conflicting information, a central element of response inhibition on flanker tasks (Botvinick M M, et al. Conflict monitoring and anterior cingulate cortex: an update. Trends Cog Sci. 2004; 8(12):539-46).

Significant correlations between tHcy level and cognitive symptoms show that tHcy level is a reliable indicator of cognitive impairment. Accordingly, cognitive symptoms in subjects having elevated tHcy levels, including non-genetically defined CBS deficient subjects, can be treated using the methods described herein to alleviate cognitive symptoms associated with elevated tHcy levels.

### Example 8. Validation of tHcy Quantification Methods

Current procedures for quantifying metabolites such as homocysteine, methionine, cysteine, and others implicated in CBS deficiency including those described in the preceding examples and Table 5 and FIG. 1, require separation of plasma from whole blood samples prior to analysis. This adds a need for sophisticated equipment and expert technical knowledge in order to analyze any given metabolite starting with a blood sample from a subject. Cheap and efficient validated methods of collecting and preparing patient blood samples for quantitative analysis are needed. Such validated methods will reduce the cost and time required to diagnose patients having CBS deficiency, for example, by enabling patients to collect blood samples at home without the need for a testing facility. These methods will also allow individual subjects and medical experts to more efficiently monitor real time disease conditions and track treatment by enabling multiple sample submissions from home. In addition, such validated diagnostics and diagnostic methods can allow subjects to reliably home-sample to monitor metabolite levels without the assistance of a medical professional. Home-sampling involves the collection and processing of a blood sample for quantitative analysis, for example, using a device such as a finger prick and a plasma separator device (PSD). The current standard for quantification of plasma total homocysteine is based on tHcy levels measured by LC-MS/MS. Accordingly, alternative diagnostic methods are needed to collect and prepare whole blood samples for analysis by LC-MS/MS. One such alternative methods involves the collection of dried blood spots (“DBS”). Alternatively, or additionally, the use of a PSD may be employed. Methods for collecting and analyzing metabolite levels using DBS and PSD approaches are described, for example, in WO2014150900 and US20120318971, the disclosures of which are incorporated herein by reference in their entirety. However, these alternative methods have never been validated, and their reliability and utility for such applications for home use such as home-sampling or home-testing by individual subjects has not been assessed. Therefore, subject total homocysteine levels were measured using the PSD method as described in WO2014150900 and compared with a traditional plasma-separated LC-MS/MS method. The results are provided in Table 10, with “PSD Method” referring to the PSD approach described in WO2014150900 and “LC-MS/MS Method” referring to the traditional plasma-separated LC-MS/MS approach.

TABLE 10
Validation of tHcy quantification using a PSD Method
Plasma tHcy (μM)

| PSD Method | LC-MS/MS Method |
|---|---|
| 173 | 118 |
| 20.6 | 31.6 |
| 10.7 | 8.9 |
| 86.6 | 139 |
| 18.2 | 13.2 |
| 169 | 94.7 |
| 13.4 | 10.2 |
| 91.1 | 82.4 |
| 55.8 | 30.1 |
| 17.2 | 12.1 |
| 53.4 | 52.6 |
| 88.8 | 61.1 |
| 104 | 58.9 |
| 140 | 133 |
| 373 | 652 |
| 44 | 31.7 |
| 104 | 83.9 |
| 402 | 491 |
| 299 | 203 |
| 391 | 364 |
| 104 | 81.1 |
| 37.4 | 32 |
| 127 | 133 |
| 66.7 | 69.2 |

Simple linear regression analysis was performed to evaluate the correlation between the samples tested using the PSD Method and the traditional LC-MS/MS Method. A best-fit line having slope of 1.096 (1/slope=0.912), with standard error of the slope of 0.0804 (95% Confidence Intervals of the slope being 0.9302 to 1.263). These results show a strong correlation and validate the PSD Method in quantifying tHcy from blood samples of individual subjects.

### Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

What is claimed is:

1. A method of treating CBS deficiency in a subject, the method comprising:
a. determining a level of a metabolic indicator of disease severity or disease progression in the subject; and
b. administering to the subject a therapeutically effective amount of a pharmaceutical formulation comprising:
i. a drug substance comprising an isolated cystathionine β-synthase (CBS) protein comprising SEQ ID NO: 1;
ii. a PEG molecule covalently bound to the CBS protein; and
iii. a pharmaceutically acceptable excipient, diluent, or adjuvant;
wherein the subject is a non-genetically defined patient having elevated tHcy levels or a non-genetically defined patient having CBS deficiency, and wherein the therapeutically effective amount of the pharmaceutical formulation comprises a dosage of the drug substance adjusted according to the level of the metabolic indicator of disease severity or disease progression in the subject.

2. The method of claim 1, wherein determining a level of a metabolic indicator of disease severity or disease progression in the subject comprises obtaining a blood or a blood plasma sample from the subject, measuring a level of one or more metabolic indicator of disease severity or disease progression in the sample, and comparing the level of the one or more metabolic indicator of disease severity or disease progression to a level of the same metabolic indicator in a control sample from a healthy subject.

3. The method of claim 1, wherein the dosage of the drug substance adjusted according to the level of the metabolic indicator of disease severity or disease progression in the subject comprises a low dose, a medium dose, or a high dose of 20NHS PEG-CBS.

4. The method of claim 3, wherein a low dose of 20NHS PEG-CBS comprises about 0.25 mg/kg to about 1.0 mg/kg 20NHS PEG-CBS.

5. The method of claim 3, wherein a medium dose of 20NHS PEG-CBS comprises about 0.5 mg/kg to about 1.5 mg/kg 20NHS PEG-CBS.

6. The method of claim 3, wherein a high dose of 20NHS PEG-CBS comprises about 1 mg/kg to about 2 mg/kg 20NHS PEG-CBS.

7. The method of claim 3, wherein a high dose of 20NHS PEG-CBS comprises about 2 mg/kg to about 10 mg/kg 20NHS PEG-CBS.

8. The method of any one of claims 1-7, wherein the metabolic indicator of disease severity or disease progression is total homocysteine (tHcy), methionine, creatinine, c-reactive protein, dimethylglycine, alanine aminotransferase, Protein C, aspartate aminotransferase (AST), anti-thrombin III, and/or apolipoprotein A.

9. The method of any one of claims 1-8, wherein the metabolic indicator of disease severity or progression is tHcy and a dosage of 20NHS PEG-CBS is administered to the subject according to an elevated-low, elevated-medium, or elevated-high tHcy level.

10. A method of improving cognitive function in a subject having elevated total plasma homocysteine (tHcy) levels, the method comprising administering to the subject a therapeutically effective amount of a pharmaceutical formulation comprising:
a drug substance comprising an isolated cystathionine β-synthase (CBS) protein comprising SEQ ID NO: 1;
a PEG molecule covalently bound to the CBS protein; and
a pharmaceutically acceptable excipient, diluent, or adjuvant.

11. The method of claim 10, further comprising providing a cognitive or behavioral intervention.

12. The method of claim 10 or 11, wherein the cognitive or behavioral intervention comprises behavioral parent training (BPT) or behavioral classroom management (BCM).

13. A method of reducing skeletal fragility in a subject having elevated total plasma homocysteine (tHcy) levels, the method comprising administering to the subject a therapeutically effective amount of a pharmaceutical formulation comprising:
a drug substance comprising an isolated cystathionine β-synthase (CBS) protein comprising SEQ ID NO: 1;
a PEG molecule covalently bound to the CBS protein; and
a pharmaceutically acceptable excipient, diluent, or adjuvant.

14. The method of claim 13, wherein skeletal fragility of the subject is assessed by bone mineral density determination.

15. The method of any one of claims 10-14, wherein the PEG molecule is ME-200GS.

16. The method of any one of claims 10-15, wherein the therapeutically effective amount comprises a dosage of about 0.25 mg/kg to about 10 mg/kg of the drug substance.

17. The method of claim 16, wherein the dosage is about 0.33 mg/kg of the drug substance.

18. The method of claim 16, wherein the dosage is about 0.66 mg/kg of the drug substance.

19. The method of claim 16, wherein the dosage is about 1.0 mg/kg of the drug substance.

20. The method of claim 16, wherein the dosage is about 1.5 mg/kg of the drug substance.

21. The method of any one of claims 10-20, further comprising administering one or more of vitamin B6, vitamin B12, folate, and betaine to the subject.

22. The method of any one of claims 10-21, wherein the subject is on a methionine (Met)-restricted diet.

23. The method of claim 22, further comprising terminating or relaxing the methionine restricted diet.

24. The method of any one of claims 10-23, further comprising administering an anti-platelet agent.

25. The method of claim 24, wherein the anti-platelet agent is a warfarin blood thinner or an anti-coagulation agent.

26. The method of any one of claims 10-25, wherein the administering a therapeutically effective amount of the pharmaceutical formulation is performed about once every 3 days.

27. The method of any one of claims 10-25, wherein the administering a therapeutically effective amount of the pharmaceutical formulation is performed about once per day.

28. The method of any one of claims 10-25, wherein the administering a therapeutically effective amount of the pharmaceutical formulation is performed about twice per day.

29. The method of any one of claims 10-25, wherein the administering a therapeutically effective amount of the pharmaceutical formulation is performed about once per week.

30. The method of any one of claims 10-25, wherein the administering a therapeutically effective amount of the pharmaceutical formulation is performed about twice per week.

31. The method of any one of claims 10-30, wherein the administering a therapeutically effective amount of the pharmaceutical formulation is repeated for about 6 weeks.

32. The method of any one of claims 10-30, wherein the administering a therapeutically effective amount of the pharmaceutical formulation is repeated for about 3 months.

33. The method of any one of claims 10-30, wherein the administering a therapeutically effective amount of the pharmaceutical formulation is repeated for about 6 months.

34. The method of any one of claims 10-30, wherein the administering a therapeutically effective amount of the pharmaceutical formulation is repeated for longer than 6 months.

35. The method of any one of claims 10-30, wherein the administering a therapeutically effective amount of the pharmaceutical formulation is repeated for the remaining life span of the subject.

36. The method of any one of claims 10-35, wherein the elevated total plasma homocysteine (tHcy) levels in the subject comprise tHcy levels greater than about 5 μmol/L.

37. The method of any one of claims 10-36, wherein the elevated total plasma homocysteine (tHcy) levels in the subject comprise tHcy levels greater than about 10 μmol/L.

38. The method of any one of claims 10-37, wherein the elevated total plasma homocysteine (tHcy) levels in the subject comprise tHcy levels greater than about 15 μmol/L.

39. The method of any one of claims 10-38, wherein the subject having elevated total plasma homocysteine (tHcy) levels is a genetically-defined HCU patient.

40. The method of any one of claims 10-38, wherein the subject having elevated total plasma homocysteine (tHcy) levels is a non-genetically defined patient having elevated tHcy levels or having CBS deficiency.


SEQUENCE LISTING (5 sequences)

SEQ ID NO: 1 - protein, 412 aa, Homo sapiens
PSETPQAEVGPTGSPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLR

SEQ ID NO: 2 - protein, 551 aa, Homo sapiens
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 3 - DNA, 1656 bp, Homo sapiens
atgccgtcagaaaccccgcaggcagaagtgggtccgacgggttgcccgcaccgtagcggtccgcattctgcaaaaggcagtctggaaaaaggttccccggaagataaagaagccaaagaaccgctgtggattcgtccggacgcaccgtcacgctgtacctggcagctgggtcgtccggcaagcgaatctccgcatcaccatacggctccggcgaaaagtccgaaaattctgccggatatcctgaagaaaattggtgacaccccgatggttcgtatcaacaaaatcggcaaaaaattcggtctgaaatgcgaactgctggctaaatgtgaatttttcaatgcgggcggttccgtgaaagatcgtatctcactgcgcatgattgaagatgctgaacgcgacggcaccctgaaaccgggtgatacgattatcgaaccgacctctggcaacacgggtatcggtctggcactggcggcggcagtccgtggttatcgctgcattatcgtgatgccggaaaaaatgagctctgaaaaagttgatgtcctgcgtgctctgggcgcggaaattgttcgtaccccgacgaatgcccgcttcgacagtccggaatcccatgtgggtgttgcatggcgcctgaaaaacgaaatcccgaattcgcacattctggatcagtatcgtaacgctagcaatccgctggcgcattacgataccacggccgacgaaatcctgcagcaatgtgatggcaaactggacatgctggtcgcttctgtgggtaccggcggtaccattacgggcatcgcgcgtaaactgaaagaaaaatgcccgggctgtcgcattatcggtgtggatccggaaggcagtattctggcggaaccggaagaactgaaccagaccgaacaaaccacgtatgaagttgaaggcatcggttacgattttattccgaccgtcctggatcgcacggtggttgacaaatggttcaaaagcaatgacgaagaagcctttaccttcgcacgtatgctgatcgctcaggaaggtctgctgtgcggtggttcagcaggttcgacggtcgcagtggcagttaaagctgcgcaggaactgcaagaaggtcaacgttgtgtcgtgattctgccggattctgttcgcaactacatgaccaaatttctgagtgaccgttggatgctgcaaaaaggcttcctgaaagaagaagatctgaccgagaaaaaaccgtggtggtggcacctgcgcgtgcaggaactgggtctgtccgcaccgctgaccgttctgccgaccatcacgtgcggccatacgattgaaatcctgcgtgaaaaaggttttgatcaggccccggttgtcgacgaagcaggcgtgattctgggtatggttaccctgggtaacatgctgagttccctgctggcgggcaaagtgcaaccgagcgatcaggttggtaaagtcatctacaaacaattcaaacagattcgtctgaccgatacgctgggccgcctgtcgcacatcctggaaatggaccatttcgcgctggttgtgcacgaacagattcaataccatagcaccggcaaatcatcgcagcgccaaatggtctttggtgtcgtgacggccattgatctgctgaatttcgtggccgcacaagaacgtgaccagaaataa

SEQ ID NO: 4 - protein, 413 aa, Artificial Sequence
  Description of Artificial Sequence Synthetic polypeptide
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLR

SEQ ID NO: 5 - protein, 413 aa, Artificial Sequence
  Description of Artificial Sequence Synthetic Truncated human C15S mutant CBS polypeptide
MPSETPQAEVGPTGSPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLR